SEQUENCE LISTING (44 sequences)

SEQ ID NO: 1 - protein, 1090 aa, Brevibacillus sp.
MPVRSFKVKLVTRSGDAEHMLQLRRGLWKTHEIVNQGIAYYMNKLALMRQEPYAGKSREVVRLELLHSLRAQQKRNNWTGDAGTDDEILNLSRRLYELLVPSAIGEKGDAQMLSRKFLSPLVDPNSEGGKGTAKSGRKPRWMKMREEGHPDWEAEREKDRAKKAADPTASILNDLEAFGLRPLFPLFTDEQKGIQWLPKQKRQFVRTFDRDMFQQALERMLSWESWNRRVAEEYQKLQAQRDELYAKYLADGGAWLEALQSFEKQREVELAEESFAAKSEYLITRRQIRGWKQVYEKWSQLPEHAAQEQFWQVVADVQTSLPGAFGDPKVYQFLSQPEHHHIWRGYPNRLFHYSDYNGVRKKLQRARHDATFTLPDPVEHPLWIRFDARGGNIHDYEISQNGKQYQVTFSRLLWPENETWVERENVTVAIGASQQLKRQIRLDGYADKKQKVRYRDYSSGIELTGVLGGAKIQFDRRHLRKASNRLADGETGPVYLNVVVDIEPFLAMRNGRLQTPIGQVLQVNTKDWPKVTGYKPAELISWIQNSPLAVGTGVNTIEAGMRVMSVDLGQRSAAAVSIFEVMRQKPAEQETKLFYPIAVTGLYAVHRRSLLLRLPGEKISDEIEQQRKIRAHARSLVRYQIRLLADVLRLHTRGTAEQRRAKLDELLATLQTKQELDQKLWQTELEKLFDYIHEPAERWQQALVAAHRTLEPVIGQAVRHWRKSLRIDRKGLAGMSMWNIEELEETRKLLIAWSKHSRVPGEPNRLDKEETFAPQQLQHIQNVKDDRLKQMANLLVMTALGYKYDEAEKQWKEAYPACQMILFEDLSRYRFALDRPRRENNRLMKWAHRSIPRLVYLQGELFGIQVGDVYSAYTSRFHAKTGAPGIRCHALKEEDLQPNSYVVKQLIKDGFIREDQTGSLKPGQIVPWSGGELFVTLADRSGSRLAVIHADINAAQNLQKRFWQQNTEIFRVPCKVTTSGLIPAYDKMKKLFGKGYFAKINQTDTSEVYVWEHSAKMKGKTTPADPAEEGVFDESLTDEMEELEDSQEGYKTLFRDPSGFFWSSDRWLPQKEFWFWVKRRIEKKLREQLQ

SEQ ID NO: 2 - DNA, 3270 bp, Brevibacillus sp.
  source 1..3270: mol_type = other DNA
atgcccgtaaggtcatttaaagttaagctagtgactcggtctggcgatgcggagcacatgctgcaactgcgtagggggctgtggaaaacccatgaaatcgtaaatcagggcattgcgtactacatgaacaagttggcgctgatgcgtcaagaaccgtatgctggtaaaagccgtgaggttgttcgtttggagctgctgcatagcctgcgtgcacagcagaaacgtaataactggaccggcgacgccggtacggatgatgaaattctgaacctgagccgtcgcctgtacgagttgctcgtgccgagcgcgatcggcgagaagggcgatgcgcagatgttatctcgcaagtttctgtcgccgctggttgatccgaattctgaaggtggtaaaggcacggcgaagtccggccgtaagccgcgttggatgaaaatgcgggaggagggtcatccggattgggaagctgaacgcgagaaggaccgcgcaaagaaagcagcggacccgactgcgtctattctgaatgatttggaagcgtttggtctgcgtccgctgttcccgctgttcaccgacgagcaaaaaggcatccagtggctgccgaaacaaaaacgtcagttcgtgcgtaccttcgatcgtgatatgtttcagcaggctctggagcgtatgctgtcctgggagtcctggaaccgccgtgtcgccgaggaatatcagaagctgcaggctcaacgtgacgagttgtacgcaaaatatctggccgatggtggtgcctggctggaagcgttacaaagctttgagaagcagcgtgaagttgaactggctgaggagtcttttgccgctaaatccgaatatctgatcacccgacgacaaattcgcggttggaaacaggtgtacgaaaagtggtcacaactgccagagcacgccgcccaagaacaattctggcaagttgtagcggacgtacagacgtctctgcctggtgcgtttggtgacccaaaagtttaccagtttctgagccagccggagcatcaccacatctggcgcggttacccgaaccgactattccactattctgattacaacggtgttcgcaagaagctgcaaagagcgcgtcacgatgcgacttttaccttgccggacccggttgaacacccgctttggatcagattcgatgcgcgtggtggtaatattcatgattatgagatcagccagaatggcaagcaatatcaagttaccttctcccgcctcctgtggcctgaaaatgaaacctgggtggagcgtgaaaacgttaccgttgcaatcggggcaagccagcagcttaagcgtcagattcggttggacggctacgccgataaaaagcagaaggtacgttatcgtgactactctagcggcatcgagctgaccggtgttctgggtggtgcgaagatccagttcgaccgtcgtcacctgcgcaaagcaagtaatcgtttagccgacggtgaaaccggccctgtgtacctgaacgtggtggtcgatatcgagccgtttctggctatgcgcaacggtcgtctacaaacgccgattggccaagtgctgcaagtcaacaccaaggactggccgaaggtgacgggttataaaccggctgagctgatcagctggattcaaaacagcccgctggcggtgggtacaggcgtcaacaccattgaggcgggtatgcgcgttatgagtgttgatctgggtcaaagaagcgcagctgctgtgagcattttcgaggtcatgcgccagaaaccggcggaacaggaaaccaaactcttctatccgattgctgtgaccggtctgtatgcggtgcatcgtcgtagtttgctgctgcgtcttccaggcgagaaaattagcgacgaaatcgaacagcaacgtaaaatcagagcccatgctcgtagcttggtgcgctaccagatccgtctgcttgcggacgtactgcgtctccacacccgtggcaccgcggaacagagacgtgcgaaactggatgaattgctggctaccctgcaaacgaaacaagagttagatcagaagttatggcaaactgagttggaaaagctgttcgactacattcacgaaccggcggagcgctggcagcaggcgctggtggcggcgcaccgtaccttggagccggttatcggtcaggcagttcgtcattggcgtaagtccctccgcattgatcgtaagggcttggcgggcatgagcatgtggaacattgaagagttggaggaaacccggaagttgttgatcgcatggagcaaacattccagggtccccggcgagccgaaccgtctggacaaagaagaaacgtttgcaccgcagcagctccaacatatccaaaacgtcaaagacgaccgcttgaagcagatggcaaatctgctcgtgatgaccgccttgggctacaagtacgatgaggccgagaaacaatggaaagaggcgtatccggcgtgtcagatgattctgttcgaagatctaagccgctaccgctttgcgctggaccgaccgcgtcgcgagaacaaccggttaatgaaatgggcacacagaagtatcccgcgtctggtttatctgcaaggtgaattgttcggcatccaggtgggtgatgtttattcggcgtatactagccgtttccatgctaaaactggtgcgccaggcattcgctgccacgcgctgaaagaagaagacctgcaaccgaacagctacgtggtgaagcaactgattaaagacggctttatccgtgaggaccagaccggttcccttaagccgggtcagattgtaccgtggtctggcggtgaactgttcgttaccttagccgaccgttccggcagccgtttggccgtgatccacgccgacatcaatgcagcgcagaatctgcaaaagagattctggcaacagaacaccgagatctttcgtgtgccgtgcaaagtgactacgtccggtctgataccggcatacgacaagatgaaaaagttattcggcaaaggctatttcgcaaagatcaaccagacggacaccagcgaagtgtacgtttgggaacacagcgctaagatgaagggcaaaacgaccccggcggacccagcagaagaaggagtttttgatgagtccctgaccgacgagatggaagagcttgaagacagccaagagggttacaagaccctgttccgtgatccgtcgggctttttttggtcgagcgatcgctggctgccgcagaaagagttctggttttgggttaaacgccgtatcgagaagaaactgcgcgagcagctccag

SEQ ID NO: 3 - DNA, 18 bp, synthetic construct
  source 1..18: mol_type = other DNA
aacacaagctttcggcag

SEQ ID NO: 4 - DNA, 22 bp, synthetic construct
  source 1..22: mol_type = other DNA
gaaatttggatctttgtcatcc

SEQ ID NO: 5 - DNA, 41 bp, synthetic construct
  source 1..41: mol_type = other DNA
tgcggccaatgtttgtaatcagccaaggaaattttggggac

SEQ ID NO: 6 - DNA, 39 bp, synthetic construct
  source 1..39: mol_type = other DNA
cgcattggcatggaagtcactttgatggcacctgtgtag

SEQ ID NO: 7 - DNA, 19 bp, synthetic construct
  source 1..19: mol_type = other DNA
ttccttgtctgattagttc

SEQ ID NO: 8 - DNA, 18 bp, synthetic construct
  source 1..18: mol_type = other DNA
accttcgggaacgtggtt

SEQ ID NO: 9 - DNA, 22 bp, synthetic construct
  source 1..22: mol_type = other DNA
atacactaattctttcacacgt

SEQ ID NO: 10 - DNA, 25 bp, synthetic construct
  source 1..25: mol_type = other DNA
cctcttattatgttagacttctcag

SEQ ID NO: 11 - DNA, 41 bp, synthetic construct
  source 1..41: mol_type = other DNA
ggaaaagaaaggtaagaacaaccctgacaaagttttcagat

SEQ ID NO: 12 - DNA, 42 bp, synthetic construct
  source 1..42: mol_type = other DNA
atggtactaagaggtttgattggaagcaaaataaacaccatc

SEQ ID NO: 13 - DNA, 24 bp, synthetic construct
  source 1..24: mol_type = other DNA
cctgagttgaatgtaaaactgagg

SEQ ID NO: 14 - DNA, 20 bp, synthetic construct
  source 1..20: mol_type = other DNA
aaccctgtcctaccatttaa

SEQ ID NO: 15 - DNA, 21 bp, synthetic construct
  source 1..21: mol_type = other DNA
acacgcctattaatttagtgc

SEQ ID NO: 16 - DNA, 22 bp, synthetic construct
  source 1..22: mol_type = other DNA
tagaaaagtcctaggttgaaga

SEQ ID NO: 17 - DNA, 42 bp, synthetic construct
  source 1..42: mol_type = other DNA
cctagtgatgttaatacctattggctccctcagggtttttcg

SEQ ID NO: 18 - DNA, 38 bp, synthetic construct
  source 1..38: mol_type = other DNA
aagttatttgactcctggtcataataagctgcagcacc

SEQ ID NO: 19 - DNA, 24 bp, synthetic construct
  source 1..24: mol_type = other DNA
caaatctaccaatggttctaaagc

SEQ ID NO: 20 - DNA, 23 bp, synthetic construct
  source 1..23: mol_type = other DNA
gattcttcttcaggttggacagc

SEQ ID NO: 21 - DNA, 23 bp, synthetic construct
  source 1..23: mol_type = other DNA
ccctacttattgttaataacgct

SEQ ID NO: 22 - DNA, 24 bp, synthetic construct
  source 1..24: mol_type = other DNA
attcttaaacacaaattccctaag

SEQ ID NO: 23 - DNA, 50 bp, synthetic construct
  source 1..50: mol_type = other DNA
tttttgtggtaataaacacccaaaaactaatgttgttattaaagtctgtg

SEQ ID NO: 24 - DNA, 45 bp, synthetic construct
  source 1..45: mol_type = other DNA
ctagtgcgaataattgcacttttgatgttttccttcaaggtccat

SEQ ID NO: 25 - DNA, 22 bp, synthetic construct
  source 1..22: mol_type = other DNA
atggatcattacaaaattgaaa

SEQ ID NO: 26 - DNA, 22 bp, synthetic construct
  source 1..22: mol_type = other DNA
atatgtctctcagccttttctt

SEQ ID NO: 27 - DNA, 22 bp, synthetic construct
  source 1..22: mol_type = other DNA
caattaattgccaggaacctaa

SEQ ID NO: 28 - DNA, 19 bp, synthetic construct
  source 1..19: mol_type = other DNA
tactgccagttgaatctga

SEQ ID NO: 29 - DNA, 41 bp, synthetic construct
  source 1..41: mol_type = other DNA
caacacgaacgtcatgatactctaagggtagtcttgtagtg

SEQ ID NO: 30 - DNA, 43 bp, synthetic construct
  source 1..43: mol_type = other DNA
actaaaatgtctgataatggaccccgggtccaccaaacgtaat

SEQ ID NO: 31 - DNA, 22 bp, synthetic construct
  source 1..22: mol_type = other DNA
aaagtcttcatagaacgaacaa

SEQ ID NO: 32 - DNA, 21 bp, synthetic construct
  source 1..21: mol_type = other DNA
aaaatcagcgaaatgcacccc

SEQ ID NO: 33 - DNA, 17 bp, synthetic construct
  source 1..17: mol_type = other DNA
ttgatgagctggagcca

SEQ ID NO: 34 - DNA, 18 bp, synthetic construct
  source 1..18: mol_type = other DNA
caccctcaatgcagagtc

SEQ ID NO: 35 - DNA, 41 bp, synthetic construct
  source 1..41: mol_type = other DNA
gtgtgaccctgaagactcggttttagccactgactcggatc

SEQ ID NO: 36 - DNA, 45 bp, synthetic construct
  source 1..45: mol_type = other DNA
cctccgtgatatggctcttcgtttttttcttacatggctctggtc

SEQ ID NO: 37 - DNA, 20 bp, synthetic construct
  source 1..20: mol_type = other DNA
atgtggatggctgagttgtt

SEQ ID NO: 38 - DNA, 20 bp, synthetic construct
  source 1..20: mol_type = other DNA
catgctgagtactggacctc

SEQ ID NO: 39 - RNA, 150 nt, synthetic construct
  source 1..150: mol_type = other RNA
gaaggtggttagctacaggctgaccagtgcagttgtgtcatgtgctacggtgacctaacacgtcactcagtcacaacggctatctatatttccactaaccaaagttagtggaaatgtagatggttagcaccgaagaacgctgaagcgctg

SEQ ID NO: 40 - RNA, 150 nt, synthetic construct
  source 1..150: mol_type = other RNA
gaaggtggttagctacaggctgaccagtgcagttgtgtcatgtgctacggtgacctaacacgtcactcagtcacaacggctatctatatttccactaaccaaagttagtggaaatgtagatggttagcacgttccatgctatctctggga

SEQ ID NO: 41 - RNA, 150 nt, synthetic construct
  source 1..150: mol_type = other RNA
gaaggtggttagctacaggctgaccagtgcagttgtgtcatgtgctacggtgacctaacacgtcactcagtcacaacggctatctatatttccactaaccaaagttagtggaaatgtagatggttagcactatgtaaagtttgaaaccta

SEQ ID NO: 42 - RNA, 150 nt, synthetic construct
  source 1..150: mol_type = other RNA
gaaggtggttagctacaggctgaccagtgcagttgtgtcatgtgctacggtgacctaacacgtcactcagtcacaacggctatctatatttccactaaccaaagttagtggaaatgtagatggttagcacgatggaaagtggagtttatt

SEQ ID NO: 43 - RNA, 150 nt, synthetic construct
  source 1..150: mol_type = other RNA
gaaggtggttagctacaggctgaccagtgcagttgtgtcatgtgctacggtgacctaacacgtcactcagtcacaacggctatctatatttccactaaccaaagttagtggaaatgtagatggttagcacgtttgtttgttcgtttagat

SEQ ID NO: 44 - RNA, 150 nt, synthetic construct
  source 1..150: mol_type = other RNA
gaaggtggttagctacaggctgaccagtgcagttgtgtcatgtgctacggtgacctaacacgtcactcagtcacaacggctatctatatttccactaaccaaagttagtggaaatgtagatggttagcacaattacttgggtgtgaccct

(19) United States
(12) Patent Application Publication
(10) Pub. No.: US 2025/0283182 A1
(43) Pub. Date: Sep. 11, 2025

(54) METHODS AND SYSTEMS FOR DETECTING A TARGET USING BRCAS12B AND GENETICALLY ENGINEERED VARIANTS THEREOF

(71) Applicant: University of Florida Research Foundation, Inc., Gainesville, FL (US)

(72) Inventors: Piyush K. Jain, Gainesville, FL (US); Long T. Nguyen, Gainesville, FL (US); Santosh Rananaware, Gainesville, FL (US)

(21) Appl. No.: 18/694,696

(22) PCT Filed: Sep. 23, 2022
(86) PCT No.: PCT/US2022/076926

§ 371 Date: Mar. 22, 2024

Related U.S. Application Data
(60) Provisional application No. 63/248,243, filed on Sep. 24, 2021.

(51) Int. Cl. C12Q 1/70; C12N 9/22; C12N 15/11; C12Q 1/34; C12Q 1/6806; C12Q 1/6823; C12Q 1/6844

(52) U.S. Cl. CPC C12Q 1/701; C12N 9/226; C12N 15/111; C12Q 1/34; C12Q 1/6806; C12Q 1/6823; C12Q 1/6844; C12N 2310/20; G01N 2333/922

(57) ABSTRACT

Novel genetically engineered thermostable Cas12b enzymes with greater thermal stability, higher melting point and/or increased trans-cleavage activity than wild type Cas 12b enzymes are provided. Methods, systems, and kits for one-pot detection of target polynucleotide are also provided that combine isothermal amplification with CRISPR-based detection with thermostable Cas enzymes in a single reaction vessel.

### RELATED APPLICATION

This application is the 35 U.S.C. § 371 national stage application of PCT Application No. PCT/US2022/076926, filed Sep. 23, 2022, which claims the benefit of and priority to U.S. provisional application entitled “METHODS AND SYSTEMS FOR DETECTING A TARGET USING BRCAS12B,” having Ser. No. 63/248,243 filed on Sep. 24, 2021, which is are entirely incorporated herein by reference.

### STATEMENT REGARDING FEDERALLY SPONSORED RESEARCH OR DEVELOPMENT

This invention was made with Government support from Grant Nos. R21 AI156321 and R35 GM147788, R21 AI168795, and R61 AI181016; awarded by the National Institutes of Health. The government has certain rights in the invention.

This invention was created in part by funding received from Grant Nos. AGR00018466—awarded by the FLORIDA BREAST CANCER FOUNDATION and USISTEF/COVID-1/247/2020—awarded by the US-INDIA SCIENCE AND TECH ENDOWMENT FUND.

### SEQUENCE LISTING

The instant application contains a Sequence Listing filed in ST.26 format entitled 222112-1350 Sequence Listing.xml created on Jan. 22, 2025, having 45,210 bytes. The content of the sequence listing is incorporated herein in its entirety.

### FIELD

The present disclosure relates to CRISPR/Cas complex-based systems and methods.

### BACKGROUND

The discovery of CRISPR/Cas (clustered regularly interspaced short palindromic repeats/CRISPR-associated) systems has provided new platforms and approaches to the field of genome engineering, diagnostics, and development of other new advanced applications in biology, agriculture, biotechnology, diagnostics, and treatment of genetic disorders. CRISPR-based diagnostics have elevated nucleic acid detection in terms of sensitivity, specificity, and rapidness.

Originally derived from various species of bacterial adaptive immune systems, the CRISPR/Cas technology works by introducing a CRISPR associated (Cas) nuclease and a short guide RNA sequence that has a region complimentary to a target sequence/site and acts as a guide by binding with Cas and directing the guide RNA/Cas complex to a target site. This complex then acts as molecular scissors to cut the target sequence at a specific site creating double-stranded cuts in the target DNA or a single-stranded cut in the target RNA. This specific target recognition and cleavage is also referred to as “cis-cleavage”. Some types of Cas proteins, once bound to a target sequence, also become active for collateral, non-specific cleavage, called “trans-cleavage.” This trans-cleavage activity can be leveraged for detection technologies.

The development of CRISPR/Cas systems for rapid, point-of-care detection of nucleic acid targets for diagnosing diseases, such as cancer and viruses, has increased recently. The SARS-CoV-2 pandemic has vastly underscored the need for developing rapid, accurate and sensitive techniques for pathogen detection. Contemporary diagnostic methods that are based on reverse transcriptase polymerase chain reaction (RT-qPCR) are widely used, but are handicapped by their dependency on expensive reagents, sophisticated equipment, and trained personnel. CRISPR-Cas systems have emerged as a widely adopted diagnostic tool for the detection of SARS-CoV-2 and other viruses and conditions within the past two years.

Class 2 type V and VI single effector Cas proteins, such as Cas12a and Cas13a, have been employed for the development of rapid, sensitive, and cost-effective detection platforms including DETECTR and SHERLOCK (Gootenberg et al., Science, 2017; Gootenberg et al., Science, 2018; Chen et al., Science, 2018; Brougton et al, Nat. Biotechnol., 2020; Young et al, NEJM., 2020) due to their robust trans-cleavage activity. The Cas12a-based DETECTR technology from Mammoth Biosciences and Cas13a-based SHERLOCK technology from Sherlock Biosciences are two CRISPR-based detection systems that are now approved by the FDA under EUA as lab-based diagnostics for detecting SARS-CoV-2 RNA. These platforms combine nucleic acid pre-amplification methods, such as RT-LAMP, RT-RPA, RT-HDA and other isothermal amplification steps, with the trans-cleavage ability of Type V and Type VI Cas effectors, for specific recognition of nucleic acid targets.

However, for the above-mentioned CRISPR-based detection methods, the pre-amplification step (such as LAMP and RT-LAMP) must be done in a separate reaction (e.g., a separate “pot”) from the CRISPR detection, since the pre-amplification has to be conducted at elevated temperatures above the melting temperature of the Cas enzyme used in the CRISPR/Cas detection step. While the optimal temperatures for RT-LAMP are around 65° C., most commercial CRISPR/Cas systems are operated at temperatures around 37° C. since many Cas enzymes become unstable at higher temperatures thereby limiting or eliminating any detection activity. The need for separate amplification and detection steps increases the time needed for the assay and can reduce sensitivity. There is a need for single pot reaction for rapid and sensitive detection of targets (e.g., diagnostics of infectious diseases, such as SARS-CoV-2).

### SUMMARY

According to various aspects, the present disclosure provides novel CRISPR associated (Cas) enzymes, and methods, systems and kits for detection of a target by in a one pot reaction and/or at elevated temperatures. The CCR systems of the present disclosure can be adapted for use with any other CRISPR-based target detection system and gene therapy application.

Embodiments of genetically engineered variant BrCas12b-associated (Cas) enzymes of the present disclosure include variant Cas12b enzymes having a peptide sequence of SEQ ID NO: 1 with at least one mutation from the group consisting of: K44W, S92L, F208W, N524V, D567W, R634L, L795I, D868I, D868V, T874H, T874S, D951F, A1015E, R159E, D209R, F208W, and combinations thereof.

Methods of the present disclosure for one-pot detection of a target polynucleotide in a sample involve combining the sample in a single reaction vessel where the reaction vessel includes the following components: a set of isothermal amplification components comprising isothermal amplification enzymes and primers configured to recognize and amplify the target polynucleotide; a BrCas12b CRISPR-associated (Cas) enzyme; an sgRNA sequence having a CRISPR RNA (crRNA) sequence configured to bind to the target polynucleotide and a tracrRNA sequence configured to interact with the BrCas12b Cas enzyme to form a CRISPR/Cas complex upon binding of the crRNA sequence to the target polynucleotide; and a plurality of probes, each probe having an oligonucleotide element labeled with a detectable label, where the probe is configured to be cleaved by the BrCas12b Cas enzyme when the crRNA sequence binds the target polynucleotide to generate a CRISPR-generated detectable signal or detectable molecule. The methods further include incubating the contents of the reaction vessel at a temperature of about 60-70° C. for a period of time and detecting the CRISPR-generated detectable signal or detectable molecule if the target polynucleotide is present in the sample. In embodiments of the methods of the present disclosure, the BrCas12b Cas enzyme included in the reaction vessel is Brevibacillus sp. SYP-B805 Cas12b having a peptide sequence of SEQ ID NO: 1 or a genetically engineered variant thereof according to the present disclosure. In embodiments of the methods of the present disclosure, the methods can further include adding other components to the reaction vessel, such as an isothermal amplification buffer, an isothermal amplification reporter, and the like.

Also provided are one-pot nucleic acid detection systems for detecting a target polynucleotide in a sample. According to embodiments, the one-pot systems of the present disclosure can include the following components: a set of isothermal amplification components comprising isothermal amplification enzymes and primers configured to recognize and amplify the target polynucleotide; a BrCas12b CRISPR-associated (Cas) enzyme; an sgRNA sequence having a CRISPR RNA (crRNA) sequence configured to bind to the target polynucleotide and a tracrRNA sequence configured to interact with the BrCas12b Cas enzyme to form a CRISPR/Cas complex upon binding of the crRNA sequence to the target polynucleotide; and a plurality of probes, each probe having an oligonucleotide element labeled with a detectable label, where the probe is configured to be cleaved by the BrCas12b Cas enzyme when the crRNA sequence binds the target polynucleotide to generate a CRISPR-generated detectable signal or detectable molecule. In embodiments of the systems of the present disclosure, the BrCas12b Cas enzyme included in the reaction vessel is Brevibacillus sp. SYP-B805 Cas12b having a peptide sequence of SEQ ID NO: 1 or a genetically engineered variant thereof according to the present disclosure. In embodiments of the methods of the present disclosure, the methods can further include adding other components to the reaction vessel, such as an isothermal amplification buffer, an isothermal amplification reporter, and the like.

The present disclosure also provides shelf stable kits for detecting a target polynucleotide in a sample, where the kits include the following: a set of lyophilized isothermal amplification components comprising isothermal amplification enzymes and primers configured to recognize and amplify the target polynucleotide; a lyophilized BrCas12b CRISPR-associated (Cas) enzyme; a lyophilized sgRNA sequence having a CRISPR RNA (crRNA) sequence configured to bind to the target polynucleotide and a tracrRNA sequence configured to interact with the BrCas12b Cas enzyme to form a CRISPR/Cas complex upon binding of the crRNA sequence to the target polynucleotide; a plurality of lyophilized probes, each probe having an oligonucleotide element labeled with a detectable label, where the probe is configured to be cleaved by the BrCas12b Cas enzyme when the crRNA sequence binds the target polynucleotide to generate a CRISPR-generated detectable signal or detectable molecule; and instructions for combining components a-d with a sample, incubating the sample at a temperature of about 60-70° C. for a period of time, and detecting the detectable signal or molecule. Kits of the present disclosure can also include a lyophilized isothermal amplification buffer, lyophilized amplification reporters, and the like. In embodiments of the kits of the present disclosure, the BrCas12b Cas enzyme can be Brevibacillus sp. SYP-B805 Cas12b having a peptide sequence of SEQ ID NO: 1 or a genetically engineered variant thereof according to the present disclosure.

Other systems, methods, features, and advantages of the present disclosure will be or will become apparent to one with skill in the art upon examination of the following drawings and detailed description. It is intended that all such additional systems, methods, features, and advantages be included within this description, and be within the scope of the present disclosure.

### BRIEF DESCRIPTION OF THE DRAWINGS

Further aspects of the present disclosure will be more readily appreciated upon review of the detailed description of its various embodiments, described below, when taken in conjunction with the accompanying drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1.1A-1.1T illustrate BrCas12b characterization and one-pot specificity & sensitivity testing. FIG. 1.1A is a schematic of a BrCAs12b/crRNA/target DNA complex illustrating cleavage pattern of dsDNA target. FIG. 1.1B illustrates a temperature-dependent in-vitro cleavage assay of AacCas12b, AapCas12b, and BrCas12b targeting more restrictive PAM TTTG. 125 nM sgRNA: 100 nM Cas12b: 7 nM dsDNA was combined in 1× NEBuffer 2.1. The experiment was repeated (n=2 biological replicates) with similar results. FIG. 1.1C is a graph of Michaelis-Menten Kinetics of BrCas12b trans-cleavage at 62° C. FIG. 1.1D illustrates differential scanning fluorimetry of apo AacCas12b, apo AapCas12b, and apo BrCas12b and their corresponding Cas12b:sgRNA complexes. FIG. 1.1E illustrates multiplexing using FITC to detect LAMP amplification and HEX to monitor trans-cleavage of BrCas12b. FIGS. 1.1F-1.1I illustrate detection capability of BrCas12b via trans-cleavage against AacCas12b and AapCas12b at various temperatures. Fluorescence kinetics within 30 minutes was monitored using HEX-based reporter. Shaded regions represent standard deviation (n=3 biological replicates). STOPCovid LAMP primers were used in FIG. 1.1F to compare performance with AapCas12b. A different set of LAMP primers (DETECTR) with optimal performance at temperature higher than 60° C. were used in FIGS. 1.1 G, 1.1H, and 1.1I. (+) denotes the presence of the SARS-CoV-2 genomic RNA control, and (−) signifies the non-template control (NTC). FIGS. 1.1J-1.1N illustrate specificity testing using a pair of sgRNA and LAMP primers targeting SARS-CoV-2 variants of concern (n=3 biological replicates). FIGS. 1.1O-T illustrate limit of detection of S gene targeting alpha (B.1.1.7), beta (B.1.352), gamma (P1), delta (B.1.617.2), omicron (B.1.1.529) and N gene, respectively (n=3 biological replicates).

FIGS. 1.2A-1.2G illustrate clinical validation of one-pot BrCas12b detection assay and portable diagnostic systems. FIGS. 1.2A-B illustrate one-pot patient sample detection. The N gene target indicates the presence of SARS-CoV-2 while non-N gene targets indicate detection of variants. Fluorescence measurements were taken at t=30 minutes. FIG. 1.2C is a graph illustrating percent accuracy of the one-pot detection at 20-, 25-, and 30-minutes targeting variants and WT SARS-CoV-2. FIG. 1.2D is a graph illustrating percent accuracy with respect to Cₜ value for SARS-CoV-2 variant detection. FIG. 1.2E is a schematic illustrating an embodiment of a one-pot assembly and comparison of a portable in-house detection instrument (FISSH) and an at-home detection method using a mobile phone with a simple lens. FIG. 1.2F illustrates FISSH footprint and dual-channel graph generated from a positive sample. FIG. 1.2G illustrates comparison of lyophilised samples to the standard one-pot reaction. Fluorescence measurements were taken at t=30 minutes and the Mean±SD (n=5 technical replicates) are indicated.

FIGS. 2.1A-2.1F illustrate aspects of structural prediction of BrCas12b. FIG. 2.1A illustrates the predicted structure of BrCas12b derived from AlphaFold and SWISS-MODEL were spatially aligned with its most homologous Cas12b family member, BthCas12b (PDBID:5WTI)³⁶. FIG. 2.1B shows schematic illustrations of BrCas12b trans-cleavage: BrCas12b forms a binary complex with its sgRNA and binds to target dsDNA (upper schematic) which activates its collateral ssDNA cleavage activity (lower schematic). FIG. 2.1C illustrates a fitness landscape of BrCas12b calculated by DeepDDG. Each tile represents computationally predicted change in the free energy (ΔΔG) relative to the wild-type BrCas12b for the 20 amino acids. FIGS. 2.1D-2.1F is a series of graphs illustrating probability of preservation of function of three residues which are likely to increase the thermostability of the BrCas12b effector calculated by Hotspot Wizard 3.0.

FIGS. 2.2A-2.2F illustrate functional characterization of embodiments of BrCas12b variants. FIG. 2.2A is a graph illustrating the change in melting temperature of BrCas12b variants compared to the wild-type effector (n=4, two technical replicates examined over two independent experiments). Error bars represent mean±SEM. FIG. 2.2B is a graph illustrating differential scanning fluorimetry plots in RFU comparing the wild-type BrCas12b for selected variants screened from (a) (n=4, two technical replicates examined over two independent experiments). FIG. 2.2C is a graph illustrating the trans-cleavage activity of BrCas12b variants performed at increasing temperatures. Heat map represents the background corrected RFU at t=15 min (n=2 biological replicates). FIG. 2.2D illustrates differential scanning fluorimetry plots in terms of derivative of RFU in FIG. 2.2B. Global minima of the curve were determined to be the melting point of the protein (n=4, two technical replicates examined over two independent experiments). FIGS. 2.2E-2.2F are graphs of prediction accuracy in terms of thermostability for SWISS-MODEL and AlphaFold models (n=4, two technical replicates examined over two independent experiments).

FIGS. 2.3A-2.3F illustrate that embodiments of engineered BrCas12b variants exhibit exceptional stability compared to wild-type BrCas12b. FIGS. 2.3A-2.3C illustrate time-dependent trans-cleavage activity at different temperatures for wild-type BrCas12b, FND (F208W/N524V/D868V) variant, and RFND (R160E/F208W/N524V/D868V) variant, respectively. Heat map represents background corrected RFU at t=15 min (n=2 biological replicates). FIGS. 2.3D-2.3E illustrate specificity testing of wild-type BrCas12b, FND variant, and RFND variant against mutated activators (n=2 biological replicates). FIG. 2.3F illustrates structural insights into mechanism of enhanced stability of embodiments of engineered BrCas12b variants at amino acid positions 208 and 868.

FIGS. 2.4A-2.4J illustrate RT-LAMP-coupled one-pot detection with embodiments of engineered BrCas12b variants. FIGS. 2.4A-2.4D are graphs illustrating one-pot detection of BrCas12b variants at 64° C., 65° C., 66° C., and 67° C., respectively (n=3 biological replicates). The variants FND, FNT, FNLD, RFND, FNPD, FNLDTA stand for F208W/N524V/D868V, F208W/N524V/T874S, F208W/N524V/L795l/D868V, R160E/F208W/N524V/D868V, F208W/N524V/P763C/D868V, F208W/N524V/L795l/D868V/T874S/A1015E, respectively. The graph in FIG. 2.4E illustrates optimization of sucrose as an additive to the one-pot reaction at 67° C. (n=3 biological replicates), and FIG. 2.4F illustrates one-pot detection reaction of BrCas12b variants with 200 mM sucrose at 67° C. (n=3 biological replicates). FIGS. 2.4G-2.4H illustrate PAM-dependent detection of RFND variant with and without RT-LAMP reaction, and FIGS. 2.4I-2.4J illustrate PAM-dependent detection of RFND variant with and without RT-LAMP reaction.

FIGS. 2.5A-2.5E illustrate clinical validation of SPLENDID in Hepatitis C infected samples. FIG. 2.5A is a schematic of the Hepatitis C RNA genome. Single-guide RNA was designed to target the 5′ untranslated region (5′ UTR) of the virus. FIG. 2.5B is a schematic illustrating detailed steps of an embodiment of a assay according to the methods/system of the present disclosure (termed “SPLENDID assay”) from patient sample extraction via a magnetic-based method to the one-pot testing. FIG. 2.4C illustrates fluorescence measurements of 80 HCV clinical samples from the SPLENDID assay showing positive, negative, false negatives, and false positives. Signal readouts were taken at t=25 minutes. FIG. 2.5D is a graph summarizing clinical validation of SPLENDID in FIG. 2.5C in terms of sensitivity, specificity, and accuracy. FIG. 2.5E is a graph illustrating receiver operating characteristic curve (ROC) of the assay at t=25 minutes.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of genetics, biochemistry, molecular biology, protein engineering, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20-25° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

All publications and patents cited in this specification are cited to disclose and describe the methods and/or materials in connection with which the publications are cited. Publications and patents that are incorporated by reference, where noted, are incorporated by reference as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. Any terms not specifically defined within the instant application, including terms of art, are interpreted as would be understood by one of ordinary skill in the relevant art; thus, is not intended for any such terms to be defined by a lexicographical definition in any cited art, whether or not incorporated by reference herein, including but not limited to, published patents and patent applications. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

Prior to describing the various embodiments, the following definitions are provided and should be used unless otherwise indicated.

### Definitions

In describing and claiming the disclosed subject matter, the following terminology will be used in accordance with the definitions set forth below.

It must be noted that, as used in the specification and the appended claims, the singular forms “a,” “an,” and “the” include plural referents unless the context clearly dictates otherwise. Thus, for example, reference to “a cell” includes a plurality of cells. In this specification and in the claims that follow, reference will be made to a number of terms that shall be defined to have the following meanings unless a contrary intention is apparent.

As used herein, “about,” “approximately,” and the like, when used in connection with a numerical variable, generally refers to the value of the variable and to all values of the variable that are within the experimental error (e.g., within the 95% confidence interval for the mean) or within +/−10% of the indicated value, whichever is greater.

The terms “comprise”, “comprising”, “including” “containing”, “characterized by”, and grammatical equivalents thereof are used in the inclusive, open sense, meaning that additional elements may be included. It is not intended to be construed as “consists of only.”

As used herein, “consisting of” and grammatical equivalent thereof exclude any element, step or ingredient not specified in the claim.

In this disclosure, “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. patent law and the term is open-ended, allowing for the presence of more than that which is recited so long as basic or novel characteristics of that which is recited is not changed by the presence of more than that which is recited, but excludes prior art embodiments.

As used herein, “cDNA” refers to a DNA sequence that is complementary to an RNA transcript in a cell. It is a man-made molecule. Typically, cDNA is made in vitro by an enzyme called reverse-transcriptase using RNA transcripts as templates.

As used herein with reference to the relationship between DNA, cDNA, cRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, “deoxyribonucleic acid (DNA)” and “ribonucleic acid (RNA)” can generally refer to any polyribonucleotide or polydeoxyribonucleotide, which may be unmodified RNA or DNA or modified RNA or DNA. RNA can be in the form of non-coding RNA such as tRNA (transfer RNA), snRNA (small nuclear RNA), rRNA (ribosomal RNA), anti-sense RNA, RNAi (RNA interference construct), siRNA (short interfering RNA), microRNA (miRNA), or ribozymes, aptamers, guide RNA (gRNA), CRISPR RNA (crRNA), Trans-activating crRNA (tracrRNA), or coding mRNA (messenger RNA).

As used herein, “gene” can refer to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA.

As used herein, the term “exogenous DNA” or “exogenous nucleic acid sequence” or “exogenous polynucleotide” refers to a nucleic acid sequence that was introduced into a cell, organism, or organelle via transfection. Exogenous nucleic acids originate from an external source, for instance, the exogenous nucleic acid may be from another cell or organism and/or it may be synthetic and/or recombinant. While an exogenous nucleic acid sometimes originates from a different organism or species, it may also originate from the same species (e.g., an extra copy or recombinant form of a nucleic acid that is introduced into a cell or organism in addition to or as a replacement for the naturally occurring nucleic acid). Typically, the introduced exogenous sequence is a recombinant sequence.

As used herein, the terms “guide polynucleotide,” “guide sequence,” or “guide RNA” (gRNA or sgRNA) as can refer to any polynucleotide sequence having sufficient complementarity with a target polynucleotide sequence to hybridize with the target sequence and direct sequence-specific binding of a CRISPR complex to the target sequence. The degree of complementarity between a guide polynucleotide and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). A guide polynucleotide (also referred to herein as a guide sequence and includes single guide sequences (sgRNA)) can be about or more than about 5, 10, 11, 12, 13, 14, 15, 16, 17, 18, 19, 20, 21, 22, 23, 24, 25, 26, 27, 28, 29, 30, 35, 40, 45, 50, 75, 90, 100, 110, 112, 115, 120, 130, 140, or more nucleotides in length. The guide polynucleotide (gRNA or sgRNA) can include a nucleotide sequence that is complementary to a target DNA sequence. This portion of the guide sequence can be referred to as the complementary region of the guide RNA or the CRISPR RNA (crRNA). Another portion of the guide sequence serves as a binding scaffold for the CRISPR-associated (Cas) nuclease. This portion of the guide sequence can be referred to as the tracrRNA. The guide sequence can also include one or more miRNA target sequences coupled to the 3′ end of the guide sequence. The guide sequence can include one or more MS2 RNA aptamers incorporated within the portion of the guide strand that is not the complementary portion. As used herein the term guide sequence can include any specially modified guide sequences, including but not limited to those configured for use in synergistic activation mediator (SAM) implemented CRISPR (Nature 517, 583-588 (29 Jan. 2015) or suppression (Cell Volume 154, Issue 2, 18 Jul. 2013, Pages 442-451).

A guide polynucleotide can be less than about 150, 125, 75, 50, 45, 40, 35, 30, 25, 20, 15, 12, or fewer nucleotides in length. The ability of a guide polynucleotide to direct sequence-specific binding of a CRISPR complex to a target sequence may be assessed by any suitable assay. For example, the components of a CRISPR system sufficient to form a CRISPR complex, including the guide polynucleotide to be tested, may be provided to a host cell having the corresponding target sequence, such as by transfection with vectors encoding the components of the CRISPR sequence, followed by an assessment of preferential cleavage within the target sequence. Similarly, cleavage of a target polynucleotide sequence may be evaluated in a test tube by providing the target sequence, components of a CRISPR complex, including the guide polynucleotide to be tested and a control guide polynucleotide different from the test guide polynucleotide, and comparing binding or rate of cleavage at the target sequence between the test and control guide polynucleotide reactions. Other assays are possible, and will occur to those skilled in the art.

As used herein, “nucleic acid,” “nucleotide sequence,” and “polynucleotide” can be used interchangeably herein and can generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphorothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

As used herein, “isolated” means separated from constituents, cellular and otherwise, in which the polynucleotide, peptide, polypeptide, protein, antibody, or fragments thereof, are normally associated with in nature. A non-naturally occurring polynucleotide, peptide, polypeptide, protein, antibody, or fragments thereof, do not require “isolation” to distinguish it from its naturally occurring counterpart.

As used herein, “polypeptides” or “proteins” refers to amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). “Protein” and “Polypeptide” can refer to a molecule composed of one or more chains of amino acids in a specific order. The term protein is used interchangeable with “polypeptide.” The order is determined by the base sequence of nucleotides in the gene coding for the protein. Proteins can be involved in the structure, function, and regulation of various functions.

As used herein with reference to the relationship between DNA, cDNA, CRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, the term “encode” refers to principle that DNA can be transcribed into RNA, which can then be translated into amino acid sequences that can form proteins

As used herein, the terms “optional” or “optionally” means that the subsequently described event or circumstance can or cannot occur, and that the description includes instances where said event or circumstance occurs and instances where it does not.

As used herein, the term “specific binding” or “preferential binding” can refer to non-covalent physical association of a first and a second moiety wherein the association between the first and second moieties is at least 2 times as strong, at least 5 times as strong as, at least 10 times as strong as, at least 50 times as strong as, at least 100 times as strong as, or stronger than the association of either moiety with most or all other moieties present in the environment in which binding occurs. Binding of two or more entities may be considered specific if the equilibrium dissociation constant, Kd, is 10⁻³ M or less, 10⁻⁴ M or less, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10⁻³ M). In some embodiments, specific binding, which can be referred to as “molecular recognition,” is a saturable binding interaction between two entities that is dependent on complementary orientation of functional groups on each entity. Examples of specific binding interactions include primer-polynucleotide interaction, aptamer-aptamer target interactions, antibody-antigen interactions, avidin-biotin interactions, ligand-receptor interactions, metal-chelate interactions, hybridization between complementary nucleic acids, etc.

As used herein, the term “recombinant” or “engineered” can generally refer to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc. Recombinant or engineered can also refer to the polypeptide encoded by the recombinant nucleic acid. Non-naturally occurring nucleic acids or polypeptides include nucleic acids and polypeptides modified by man.

As used herein, “variant” can refer to a polynucleotide or polypeptide that differs from a reference polynucleotide or polypeptide, but retains essential and/or characteristic properties (structural and/or functional) of the reference polynucleotide or polypeptide. A typical variant of a polypeptide differs in amino acid sequence from another, reference polypeptide. The differences can be limited so that the sequences of the reference polypeptide and the variant are closely similar overall and, in many regions, identical. A variant and reference polypeptide may differ in nucleic or amino acid sequence by one or more modifications at the sequence level or post-transcriptional or post-translational modifications (e.g., substitutions, additions, deletions, methylation, glycosylations, etc.). A substituted nucleic acid may or may not be an unmodified nucleic acid of adenine, thiamine, guanine, cytosine, uracil, including any chemically, enzymatically or metabolically modified forms of these or other nucleotides. A substituted amino acid residue may or may not be one encoded by the genetic code. A variant of a polypeptide may be naturally occurring such as an allelic variant, or it may be a variant that is not known to occur naturally. “Variant” includes functional and structural variants.

As used herein, “organism”, “host”, and “subject” refers to any living entity comprised of at least one cell. A living organism can be as simple as, for example, a single isolated eukaryotic cell or cultured cell or cell line, or as complex as a mammal, including a human being, and animals (e.g., vertebrates, amphibians, fish, mammals, e.g., cats, dogs, horses, pigs, cows, sheep, rodents, rabbits, squirrels, bears, primates (e.g., chimpanzees, gorillas, and humans).

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents and are meant to include future updates.

Reference throughout this specification to “one embodiment”, “an embodiment”, “another embodiment”, “some embodiment,” means that a particular feature, structure or characteristic described in connection with the embodiment is included in at least one embodiment of the present invention. Thus, appearances of the phrases “in one embodiment,” “in an embodiment,” “in another embodiment”, or “in some embodiment” in various places throughout this specification are not necessarily all referring to the same embodiment, but they may. Furthermore, the particular features, structures or characteristics may be combined in any suitable manner, as would be apparent to a person skilled in the art from this disclosure, in one or more embodiments. Furthermore, while some embodiments described herein include some, but not other, features included in other embodiments, combinations of features of different embodiments are meant to be within the scope of the invention. For example, in the appended claims, any of the claimed embodiments can be used in any combination.

### Discussion

In accordance with the purpose(s) of the present disclosure, as embodied and broadly described herein, embodiments of the present disclosure, in some aspects, relate to compositions, systems and methods of detecting a target polynucleotide from a sample in a single-pot as well as novel Crisper associate (Cas) enzymes with increased thermal stability and/or activity. The systems and methods of the present disclosure combine novel Cas enzymes with activity at higher temperatures, as well as methods and systems for isothermal amplification and CRISPR/Cas detection in a single pot at elevated temperatures for rapid and accurate detection of target sequences. The present disclosure also includes kits for use in the methods of the present disclosure. According to some aspects, the present disclosure provides novel Cas enzymes, methods, systems, and kits for rapid and accurate detection of infectious agents, including, but not limited to, conditions such as to cancer, infectious diseases (including, without limitation, viruses such as SARS-CoV-2, HIV, and the like). In some embodiments, the methods, systems, and kits of the present disclosure are able to detect/distinguish different variants of SARS-CoV-2. Additional aspects of the current disclosure are provided in the discussion below.

### Overview

The breakthrough of CRISPR/Cas (clustered regularly interspaced short palindromic repeats/CRISPR-associated) systems has transformed the slow-progressing field of genome engineering with diverse applications in biology, agriculture, biotechnology, diagnostics, and treatment of genetic disorders. Originally derived from adaptive immune systems of different species of bacteria, the initial iterations of CRISPR/Cas9 technology enabled genetic engineering by providing targeted endonuclease activity at sites determined by a combination of the PAM (protospacer adjacent motif) requirement of each specific Cas enzyme complexed with a sgRNA (single guide RNA) composed of a variable length crRNA sequence complementary to the target site fused to a Cas specific invariant tracrRNA sequence. For Cas9 enzymes, this complex then creates double-stranded cuts in the DNA or a single-stranded cut in the RNA. This specific target recognition and cleavage is also referred to as ‘cis-cleavage’.

Some CRISPR/Cas systems also exhibit collateral non-specific cleavage or ‘trans-cleavage’ of single-stranded nucleic acids, which is activated immediately after the specific target recognition or ‘cis-cleavage’ of the target. CRISPR is currently classified into two classes based on whether they require multiple Cas effector proteins (Class 1) or a single Cas effector (Class 2). Class 1 includes systems that require multiple Cas effector proteins. Class 2 systems are of high interest as they are based on single Cas effector proteins and crRNA and are further divided into type II, V, and VI. Type II includes, Cas9, which has been studied for gene editing applications. The type V and VI CRISPR/Cas systems such as CRISPR/Cas12, CRISPR/Cas13, and CRISPR/Cas14 (abbreviated as CRISPR/Cas12-14) are emerging as powerful tools for nucleic acid detection and applications in gene and RNA editing.

Both type V and VI systems are of special interest as they are based on single Cas effector proteins and can cleave dsDNA and ssRNA, some without requiring a longer tracrRNA, and they possess a trans-cleavage activity that can be applied for diagnostics. Using single-stranded nucleic acid-based FRET reporters, the trans-cleavage activity of type V and VI systems has been applied for the detection of target dsDNA and ssRNA targets at low nM (1-10 nM) concentrations without further amplification. However, no trans-cleavage signal could be observed below 10 nM of dsDNA without any DNA amplification using the Acidaminococcus sp. derived AsCas12a or below 1 nM (100 fmols) using Lachnospiraceae bacterium derived LbCas12a. However, by coupling these detection systems with isothermal amplification, low aM concentration of cell-free tumor DNA in lung cancer and viral RNA in human saliva and blood samples have been achieved.

The BrCas12b CRISPR/Cas enzyme is a thermostable protein from Brevibacillus species with one of the fastest reported kinetics for cis cleavage activity of DNA; however, no one has reported its trans-cleavage activity or use in nucleic acid detection (https://pubmed.ncbi.nlm,nih.gov/31926228/). For the first time, the present disclosure describes that BrCas12b also has a high trans-cleavage activity after recognizing a target. In fact, as demonstrated in the examples below, BrCas12b appears to be one of the fastest CRISPR/Cas enzymes in terms of trans-cleavage activity. Due to its thermophilicity, the BrCas12b enzyme is stable at high temperatures (such as 60-70° C.). The enzyme's kinetics increase at higher temperatures than most Cas enzymes, along with the rate of cleavage reaction, possibly due to enhanced diffusion rate of substrates, allowing rapid detection of nucleic acids at high temperatures. The BrCas12b enzyme isolated from an unclassified Brevibacillus species identified as SYP-B805 is particularly thermostable with high activity. The peptide sequence of BrCas12b is SEQ ID NO: 1, which is encoded by nucleotide sequence SEQ ID NO: 2.

As described in greater detail in the examples below, genetic variants of BrCas12b with one or more mutations have been engineered that have even greater thermal stability and/or activity than wild type BrCas12b. Methods, systems, and kits of the present disclosure employ BrCas12b enzymes (including, but not limited to, wild type BrCAs12b enzymes and/or the engineered genetically modified variants thereof of the present disclosure), for detection of nucleic acid targets in a sample at higher temperature with an isothermal amplification step (e.g., LAMP, RT-LAMP) for detection of polynucleotide targets (including, but not limited to RNA viruses such as SARS-CoV-2) in a single pot reaction. The novel BrCas12b enzymes, as well as the methods, systems, and kits including BrCas12b enzymes will be described in greater detail below.

The BrCas12b enzymes of the present disclosure can also be used in other CRISPR-based technologies. For example, the BrCas12b enzymes can be used in conjunction with other Cas enzymes. For instance, specificity of enzymes for a target can also be enhanced at higher temperatures, and several Cas12a variants have been identified that can function at different temperatures (see, for instance, https://www.medrxiv.org/content/10.1101/2021.07.21.21260653v1, which is hereby incorporated by reference herein). Therefore, in some embodiments, BrCas12b can also be combined and/or multiplexed with other Cas enzymes (such as Cas12a) to detect two different targets at different temperatures. For example, the activity of Cas12b can be blocked by incorporating an inhibitor (e.g., an anti-CRISPR or blocked sgRNAs). The inhibitor can be configured to be removed or degraded at higher temperatures, such as above 60° C., above 62° C., or at or above 65° C., thereby activating the BrCas12b enzyme for detection of a target.

The BrCas12b enzyme, like various Cas12a enzymes, appears to be compatible with other CRISPR/Cas technologies. This includes CRISPR Chain Reaction technologies, where two or more CRISPR/Cas complexes are combined in a chain reaction, where one complex is activated and can activate a second inactive CRISPR/Cas complex. Such systems are described in greater detail in PCT/US2021/034971, which is hereby incorporated by reference herein. In some embodiments, a BrCas12b enzyme can be employed in such systems and methods, such as combined with itself or other Cas12a enzymes (such as, but not limited to ArCas12a, AsCas12a, BfCas12a, BoCas12a, BsCas12a, CMaCas12a, CmtCas12a, ErCas12a, FnCas12a, HkCas12a, LbCas12a, Lb2Cas12a, Lb5Cas12a, MbCas12a, Mb2Cas12a, Mb3Cas12a, MiCas12a, Pb2Cas12a, PcCas12a, PdCas12a, PrCas12a, PxCas12a, TsCas12a), other Cas12b enzymes (such as, but not limited to, AapCas12b, AacCas12b, BhCas12b, AkCas12b, EbCas12b, LsCas12b, BthCas12b, BvCas12b, AaCas12b, and the like), Cas12a enzymes, and/or Cas13b enzymes. Such combined systems can create an exponential amplification chain reaction with increased specificity and sensitivity, including elevated temperature detection.

In yet other embodiments, the BrCas12b enzyme can also be combined in a multiplexed fashion with different Cas enzymes based on sequence cleavage preferences, multiplexing based on temperature, or combined with aptamers for detecting small molecules. All such systems can be incorporated with a variety of reporter systems based on one or more of fluorescence, luminescence, color change, product formation redox reaction, pH change, surface reaction or cleavage, change in electrical conductivity, resistance, and/or impedance.

As described in greater detail in the Examples and figures below, embodiments and variations of the one-pot CRISPR/Cas detection systems, methods, and kits include various elements that can be combined in a single-pot for quick and accurate detection of a target polynucleotide in a sample. Some elements of the methods, systems, and kits are described below.

### Genetically Modified BrCas12b Cas Variants

As discussed above, a type V-B CRISPR-associated enzyme from an unclassified Brevibacillus species (designated as SYP-B805) was discovered to have higher thermal stability than other Cas12a and Cas12b enzymes previously identified. This SYP-B805 Cas12b has a polypeptide sequence of SEQ ID NO: 1 and a nucleotide sequence of SEQ ID NO: 2. This stability of the BrCas12b enzyme at higher temperatures and higher melting point provides advantages for applications that need to be performed at higher temperatures than the typical melting points of other Cas enzymes. Further engineering was performed on the BrCas12b enzyme to generate structure-guided, site-specific point mutations in different domains within the protein with the goal of modifying the thermal stability and/or ssDNA and/or dsDNA cleaving activity of the enzyme. As described in Example 2, below, site directed mutagenesis was performed on the BrCas12b enzyme having peptide sequence SEQ ID NO: 1 to generate specific point mutations within different domains of the enzyme. These genetically modified variants were tested for thermal stability, increased trans cleavage activity and other properties.

It was found that several of the mutants had improved thermal stability and a higher melting temperature than the wild type BrCas12b, and some of the mutants had increased cleavage activity. Some mutations that provided increased thermostability and higher melting point include, but are not limited to: K44W, S92L, F208W, N524V, D567W, R634L, L795I, D868I, D868V, T874H, T874S, D951F, A1015E, and combinations thereof. Mutations that provided increased cleaving efficiency via increased nuclease activity (e.g., cis and/or trans cleavage) include, but are not limited to, R159E, D209R, F208W, and combinations thereof. Embodiments of the present disclosure include genetically engineered variant BrCas12b enzyme including a peptide sequence of SEQ ID NO: 1, except that it has one or more of the following point mutations: K44W, S92L, F208W, N524V, D567W, R634L, L795I, D868I, D868V, T874H, T874S, D951F, A1015E, R159E, D209R, F208W, and various combinations of these point mutations, including 2 or more, 3 or more, 4 or more, and so forth. In embodiments, genetically engineered variants having 2 or more of the above mutations can have both increased thermal stability as well as increased cleaving activity as compared to standard Cas enzymes and even the unmodified, wild type BrCas12b enzyme. In embodiments, the genetically engineered variants have higher melting temperatures (at which the protein becomes structurally unstable and denatures) than other Cas enzymes, such as other Cas12a and Cas12b enzymes, including wild type BrCas12b. The genetically engineered BrCas12b enzymes maintain structural stability and enzymatic activity at temperatures above 55° C., including, in some embodiments, at temperatures ranging from about 55 to about 70° C.

These engineered variant thermostable BrCas12b enzymes can be used in applications that require activity at elevated temperatures. For instance, these high temperature Cas variants can be efficiently coupled with nucleic acid pre-amplification techniques, such as isothermal amplification techniques, including but not limited to Loop Mediated Isothermal Amplification (LAMP) and reverse-transcription LAMP (RT-LAMP), which are performed at temperatures ranging from 55-70° C. The thermostable Cas12b enzymes of the present disclosure can also be used in genome editing applications where a high temperature endonuclease is employed, such as, for example, in gene editing a thermophilic bacterial strain that grows at temperatures above 55° C.

In addition, stability at higher temperatures also can indicate that the Cas enzymes have increased lifetime for applications at lower temperatures. Applications that would benefit from increased lifetime of Cas enzymes include, but are not limited to applications such as genome editing in human plasma, which is at a temperature of about 37° C. While other Cas enzymes may be active at this temperature, their lifespan is often limited, but these Cas enzymes with enhanced thermostability can have a longer lifespan at normal temperatures than Cas with standard thermostability, which is advantageous for some genome editing applications.

In embodiments, the hyperactive Cas12B enzymes (engineered variant Cas12b enzymes with increased cleaving activity relative to wild-type enzymes) can be used in efficient genome editing applications as well as nucleic acid detection applications. In some embodiments, the genetically engineered variant Cas12b enzymes have both increased thermostability and increased cleavage efficiency relative to other wild type Cas12b enzymes, including wild type BrCas12b.

The genetically engineered variant BrCas12b enzymes of the present disclosure can be used in the methods, systems, and kits of the present disclosure described in greater detail below.

### One-Pot BrCas12b CRISPR/Cas Nucleic Acid Detection Systems and Methods:

In embodiments, the one-pot nucleic acid detection systems of the present disclosure include one-pot nucleic acid detection systems for detecting a target polynucleotide in a sample. The systems and methods of the present disclosure combine isothermal amplification approaches and CRISPR/Cas detection to detect a target polynucleotide in a one-pot reaction. The isothermal amplification elements of the methods and systems include a set of isothermal amplification components including isothermal amplification enzymes (e.g., polymerases, reverse transcriptase, etc., as needed) and isothermal amplification primers configured to recognize and amplify the target polynucleotide. The CRISPR/Cas detection portion of the systems and methods include a thermostable BrCas12b Cas enzyme, a single guide RNA (sgRNA) sequence having a CRISPR RNA (crRNA) sequence configured to bind to the target polynucleotide of interest, and probes configured to be cleaved by the BrCas12b enzyme when the crRNA sequence binds the target polynucleotide in the sample. Some of these elements and additional elements will be described in greater detail below as well as in the examples and figures. The present disclosure also provides kits that include shelf stable components of the systems of the present disclosure that can be used in the methods of the present disclosure, as described in greater detail below.

### Isothermal Amplification Platforms and Components

Methods, systems and kits of the present disclosure include a set of isothermal amplification components, including, but not limited to isothermal amplification primers and isothermal amplification enzymes.

Isothermal amplification platforms are platforms that allow for amplification of a target polynucleotide in a sample without the high-temperature thermocycling and expensive equipment required for PCR amplification. Isothermal amplification is performed at moderately high temperatures of above 55° C., such as from about 60-70° C. (typically about 60-65° C.) which can be maintained in relatively simple and readily available equipment, including a warm water bath. The reaction components for isothermal amplification include sets of isothermal amplification primers and thermostable enzymes, such as polymerases, and in the case of RNA amplification reverse transcriptase for converting RNA targets to DNA for amplification. The isothermal amplification primers include sets of primers for recognizing, binding, an initiating amplification of the target polynucleotide.

Common platforms for isothermal amplification include loop-mediated isothermal amplification (LAMP) and reverse-transcription LAMP (RT-LAMP). LAMP is typically used for amplification/detection of DNA and includes thermostable DNA polymerases, where RT-LAMP is used for amplification/detection of RNA and includes both thermostable reverse transcriptase and thermostable DNA polymerase. Both LAMP and RT-LAMP include sets of LAMP primers. LAMP primers typically include a set of 4 to 6 thermostable primers configured to bind to different small target sequences of the target polynucleotide to initiate amplification. In embodiments the LAMP primers include at least a forward internal primer (FIP), a backward internal primer (BIP), a forward outer primer (sometimes referred to as F3), and a backward outer primer (sometimes referred to as B3). In embodiments, the LAMP primers can also include a set of loop primers. LAMP primers for specific target polynucleotides can be designed using software programs such as described in the examples below. In embodiments, the target polynucleotide can be a DNA or RNA associated with a specific condition/disease, such as cancer, genetic disease, or infectious agent (e.g., bacteria, virus, fungal, etc.). In embodiments, the target polynucleotide is a virus, such as SARS-CoV-2, HIV, HCV, Chagas, malaria, and the like. LAMP primers can be designed for recognition and amplification of any such DNA or RNA targets. Additional description of isothermal amplification enzymes and primers, such as LAMP/RT-LAMP primers and enzymes, is provided in the examples below. Sequences of some example embodiments of some LAMP/RT-LAMP primer are described below.

In embodiments, the isothermal components in the reaction vessel also include an isothermal amplification buffer compatible with the isothermal amplification components and the BrCas12b Cas enzyme. The wild type and genetically engineered BrCas12b enzymes of the present disclosure have been found to be fairly universal and compatible with various isothermal amplification buffers. In embodiments, the buffer is a buffer is a LAMP Master Mix, such as but not limited to buffers from NEB and ThermoFisher. In embodiments the buffer can be, but is not limited to one of the following: NEBuffer 2.1, WarmStart® Multi-Purpose LAMP/RT-LAMP 2× Master Mix (with UDG) (“NEB LAMP Master Mix”), and the SuperScript IV RT-LAMP Master Mix from ThermoFisher (Catalog #A51801).

In some embodiments of the present disclosure, the set of isothermal amplification components also includes an isothermal amplification reporter that produces a detectable signal upon amplification of the target sequence, such that the signal becomes detectable after sufficient amplification of the target sequence, indicating its presence in the sample. The isothermal amplification reporter is configured to produce an amplification-generated detectable signal or detectable molecule upon amplification of the target polynucleotide. In embodiments, the amplification-generated detectable signal or molecule of the isothermal amplification reporter is different and distinguishable from the CRISPR-generated detectable signal or detectable molecule produced by cleavage of the probes (described in greater detail below). In embodiments, the isothermal amplification reporter is an amplification-generated dye or other detectable signal. In embodiments, the isothermal amplification reporter is a SYTO dye, including but not limited to SYTO9, SYTO62, SYTO17, SYTO59, and SYTO60-64.

In some embodiments of systems of the present disclosure, the system further includes a single reaction vessel to contain the elements of the system of the present disclosure in a single pot. The system can also include, in embodiment, a heating element to maintain the reaction vessel at the reaction temperature for isothermal amplification and detection with the BrCas12b enzymes of the present disclosure. In embodiments the heating element is configured to maintain the temperature of the reaction vessel at a temperature above about 55° C., above about 60° C., above about 63° C., in a range of about 60-70° C., or other intervening temperatures or ranges as appropriate.

### Crispr-Associated (Cas) Enzyme

CRISPR-associated (Cas) enzymes (also known as CRISPR effector protein) are enzymes which can bind to a guide RNA (sgRNA) and to a complementary target polynucleotide sequence, forming a CRISPR/Cas complex, and can cleave the target sequence (cis cleavage). Some Cas enzymes possess both cis- and trans-cleavage activity, where trans-cleavage activity is activated upon binding of the CRISPR/Cas complex with the target sequence. Activation of the trans cleavage activity allows cleavage of probes also included in the reaction mixture, such that the probes produce a detectable signal or molecule that indicates the presence of the target sequence.

In embodiments of the present disclosure the Cas enzymes are from a hot spring bacterium Brevibacillus or are one or more of the genetically engineered variants thereof of the present disclosure described above. The Brevibacillus Cas enzyme, BrCas12b, and the engineered variants, exhibit outstanding stability at high temperatures (up to 70° C.) and, as described in the Examples below, were found to exhibit robust trans cleavage activity at these elevated temperatures, which is suitable for coupling with an isothermal amplification reaction, such as LAMP and/or RT-LAMP.

Some types of Cas12b enzymes have been shown to have high rates of cis-cleavage at somewhat elevated temperatures, but it is known in the field that trans cleavage activity does not always directly correspond to cis-cleavage activity. While AapCas12b and AacCas12b have been shown to have trans-cleavage activity, and have been combined with RT-LAMP, they are sub-optimal because the melting temperatures of these Cas enzymes (55.3° C., 58.3° C., respectively) are below the typical operating temperatures for RT-LAMP (typically around 60-65° C.), meaning that these Cas enzymes start degrading at temperatures just below 60° C. therefore reducing the enzymatic activity and requiring a much longer time to detect target RNA in a sample (typically around 45 minutes for accurate detection). Additionally, specificity of crRNA binding to the target polynucleotide is reduced at lower operating temperatures. BrCas12b, from Brevibacillus sp. SYSU G02855 (protein sequence SEQ ID NO: 1 and nucleotide sequence SEQ ID NO: 1) has been shown to have high rates of cis-cleavage https://pubmed.ncbi.nlm.nih.gov/31926228/, but there are no published studies on trans-cleavage rates. Example 1 of the present disclosure demonstrate that BrCas12b has a high melting point (of about 63.4° C.), as shown by enzyme kinetics, and exhibits high rates of trans-cleavage activity, with significantly higher enzymatic activity at temperatures above 60° C. than either AapCas12b or AAcCas12b and higher specificity of detection, with the ability to discriminate different variants of SARS-CoV-2. These features allow BrCas12b enzyme to be used in a single-pot CRISPR/Cas-based detection test combined with isothermal amplification (e.g., LAMP and RT-LAMP) that can detect a target nucleic acid both accurately and rapidly.

In embodiments, the BrCas12b CRISPR-associated (Cas) enzyme of the systems and methods of the present disclosure is derived from subspecies Brevibacillus sp. SYP-B805 (GenBank ID: WP_165214399.1, SEQ ID Nos: 1 and 2). However, further studies, as described in Example 2 below, were conducted to generate structure-guided, site-specific modifications to the BrCas12b enzyme to improve the thermal stability and/or the cleaving activity of the enzyme at temperatures above the melting temperature of wild type BrCas12b. The resulting engineered variant BrCas12b enzymes having one or more site specific point mutations in different domains within the protein have improved thermal stability, higher melting temperatures, and/or increased cleaving activity (cis and/or trans cleavage). As described above, embodiments of the genetically engineered variant BrCas12b enzymes can have one or more point mutations including, but not limited to, K44W, S92L, F208W, N524V, D567W, R634L, L795I, D868I, D868V, T874H, T874S, D951F, A1015E, R159E, D209R, F208W, and combinations thereof.

In embodiments, the genetically engineered BrCas12b enzymes of the present disclosure can have structural stability and exhibit trans cleavage activity at temperatures up to about 70° C., and even in some cases up to about 75° C. (with addition of sucrose), with robust activity between about 60 and 68° C.) and a melting temperature ranging from about 65 to 70° C., such as 65-68° C. This compares favorably to the melting temperatures of AacCas12 b and AapCas12b, which have melting temperatures of 55.3° C., 58.3° C., respectively, (and even to wild type BrCas12b, with a melting temperature of about 63.4° C.) and have a much lower activity at the temperatures used for LAMP, RT-LAMP and other isothermal amplification platforms.

sgRNA and crRNA

The guide polynucleotide, also called guide RNA or sgRNA, of the present disclosure includes both a guide CRISPR RNA (crRNA) sequence and a conserved sequence (tracrRNA). The crRNA sequence is configured to bind to a target polynucleotide, and the tracrRNA sequence is conserved among sgRNA from closely related bacterial species and is configured to act as a scaffold for complexing with the BrCas12b Cas enzyme upon binding of a target sequence to form a CRISPR/Cas complex.

In some embodiments, a sgRNA (crRNA+tracrRNA) is about 130 base pairs. In embodiments, the length of the guide sequence of the crRNA is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, the guide sequence is less than about 75, 50, 45, 40, 35, 30, 25, 20, 15, 12, or fewer nucleotides in length. In one embodiment, the guide sequence is 10⁻³⁰ nucleotides long.

In embodiments, the target polynucleotide can be a DNA or RNA associated with a specific condition/disease, such as cancer, genetic disease, or infectious agent (e.g., bacteria, virus, fungal, etc.). In embodiments, the target polynucleotide is a virus, such as, but not limited to SARS-CoV-2, HIV, HCV, Chagas, malaria, and the like. Thus, in embodiments the crRNA can be complimentary to and configured to bind a complimentary region on the target DNA or RNA of interest to detect the specific condition/disease, such as cancer, genetic disease, infection agent (e.g., bacteria, virus, etc.) and the like. The target polynucleotide in the sample is amplified by the isothermal amplification components, and then the CrRNA binds the amplified target polynucleotides and forms a CRISPR/Cas complex with the thermostable BrCAS12b to activate the trans cleavage activity of the BrCas12b, which cleaves probes in the reaction vessel to create a detectable signal and indicate the presence of the target polynucleotide in the sample, thereby diagnosing the specific condition/disease associated with the target polynucleotide.

In embodiments, such as described in the examples below, the target polynucleotide is from a virus such as, but not limited to, SARS-CoV-2, HIV, HCV, Chagas, malaria, etc., and the CRSPR/BrCas12b complex binds the target polynucleotide, activating trans cleavage activity, cleaving the probes, and detecting the presence of the virus in the sample. In some embodiments the target polynucleotide is a specific for a variant of a virus, such as variants of SARS-CoV-2, and the specificity of the crRNA is specific enough to distinguish between variants of SARS-CoV-2, such as, but not limited to Alpha (B.1.1.7), Beta (B.1.352), Delta (B.1.617.2), and Gamma (P1). Example sequences of crRNA for detection of SARS-CoV-2, including different variants, are provided in the tables below.

### Probe

As used herein, a “probe” refers to a polynucleotide-based molecule that can be cleaved by an activated CRISPR-associated (Cas) enzyme with a trans-cleavage activity to produce a detectable signal or a detectable molecule. A detectable signal may be any signal that can be detected using optical, fluorescent, chemiluminescent, electrochemical or other detection methods known in the art. The probe comprises an oligonucleotide element. In one embodiment, a first end of the oligonucleotide element in the probe is linked to a fluorophore; and a second end of the oligonucleotide element in the probe is linked to a quencher of the fluorophore. In one embodiment, the probe further comprises biotin. In some embodiments of the present disclosure, the probes are configured to be cleaved by a BrCas12b enzyme of the present disclosure that is in crRNA/Cas complex (e.g., an activated BrCas12b enzyme), such that the detectable signal or molecule can be produced upon binding of the crRNA/BrCAs12b complex to the target polynucleotide.

Quenching of the fluorophore can occur as a result of the formation of a non-fluorescent complex between the fluorophore and another fluorophore or nonfluorescent molecule. This mechanism is known as ground state complex formation, static quenching, or contact quenching. Accordingly, the oligonucleotide element may be designed so that the fluorophore and quencher are in sufficient proximity for contact quenching to occur. Fluorophores and their cognate quenchers are known in the art and can be selected for this purpose by one having ordinary skill in the art.

Upon activation of the BrCas12b enzyme disclosed herein (e.g., by recognition of the target sequence and formation of an active crRNA/Cas complex), the oligonucleotide-based probe is cleaved, thereby severing the proximity between the fluorophore and quencher needed to maintain the contact quenching effect. Accordingly, detection of the fluorophore may be used to determine the presence of a target molecule in a sample. In one embodiment, the fluorophore is selected from the group consisting of FITC, HEX and FAM, and the quencher is selected from the group consisting of BHQ1, BHQ2, MGBNFQ, and 3IABKFQ. In one embodiment, a first end of the oligonucleotide element in the probe is linked to a fluorophore; a second end of the oligonucleotide element in the probe is linked to a quencher; and the probe further comprises biotin. In embodiments, the probe is selected from HEX-polyT-Quencher (HEX-FQ) and FAM-polyT-Quencher, which are shown in the examples below to work well in the one pot assays of the present disclosure. In one embodiment, a fluorophore-quencher probe is within the crRNA.

A detectable molecule may be any molecule that can be detected by methods known in the art. In one embodiment, the detectable molecule is one member of a binding pair and can be detected by binding to another member of the binding pair. Examples of binding pairs include, but are not limited to, antibody-antigen pairs, enzyme-substrate pairs, receptor-ligand pairs, and streptavidin-biotin.

In one embodiment, the oligonucleotide element in the probe is ssDNA, since BrCas12b trans cleavage activity preferentially cuts ssDNA. Since Cas12 enzymes preferentially cleaves DNA with an A/T rich sequence, in embodiments the oligonucleotide element of the probe includes at least 55%, 60%, 65%, 70%, 75%, 80%, 90%, or 95% of A and/or T. In embodiments the oligonucleotide element of the probe is a ssDNA and is about 80% of A and/or T. In some embodiments the oligonucleotide element is TA-rich or TA-only, and is about 2-10 nucleotides in length. In one embodiment, the ssDNA of the oligonucleotide element of the probe consists of A and/or T. In one embodiment, the oligonucleotide element is TTATT. In some embodiments, the oligonucleotide element is primarily or only T “poly T”, and in some embodiments it is polyT and about 2-10 nucleotides in length. In embodiments it is an “8-mer poly T” (TTTTTTTT).

In one embodiment, the probe can be, but is not limited to a HEX-FQ reporter or a FAM-FQ reporter, such as HEX-TTATT-FQ or FAM-TTATT-FQ. In another embodiment, the probe comprises HEX or FAM-polyT-Quencher (HEX/FAM-FQ). In embodiments, the probe can also be and FITC probe or a Cy5 probe, such as, but not limited to FITC-polyT-Quencher and Cy5-PolyT-quencher.

### Methods

The present disclosure also includes one-pot methods of detecting a target polynucleotide in a sample, by combining the isothermal amplification/CRISPR/Cas system elements described above with a sample in a single reaction vessel, incubating the contents of the reaction vessel at a temperature of about 60-70° C. (or any intervening temperature or range) for a period of time and detecting the CRISPR-generated detectable signal or detectable molecule if the target polynucleotide is present in the sample. In embodiments, the contents of the reaction vessel are incubated at a temperature of about 60-65° C. In embodiments, the contents of the reaction vessel are incubated at a temperature of about 62-70° C. The period of time until a signal is detected can vary and may be from about 5 minutes to about 45 minutes. In embodiments, the contents of the reaction vessel are incubated for about 5-45 minutes, such as from about 10⁻³⁰ minutes, about 15-30, minutes, and other intervening and overlapping ranges.

As described above, the elements (as described above) included in the reaction vessel with the sample include: a set of isothermal amplification components including isothermal amplification enzymes and primers configured to recognize and amplify the target polynucleotide; a BrCas12b CRISPR-associated (Cas) enzyme; an sgRNA sequence having a CRISPR RNA (crRNA) sequence configured to bind to the target polynucleotide and a tracrRNA sequence configured to interact with the BrCas12b Cas enzyme to form a CRISPR/Cas complex upon binding of the crRNA sequence to the target polynucleotide; and a plurality of probes, each probe having an oligonucleotide element labeled with a detectable label, wherein the probe is configured to be cleaved by the BrCas12bCas enzyme when the guide sequence binds the target polynucleotide to generate a CRISPR-generated detectable signal or detectable molecule.

In some embodiments, the BrCas12b is Brevibacillus sp. SYP-B805 Cas12b, such as BrCas12b having the peptide sequence SEQ ID NO: 1. In some embodiments the BrCas12b is a genetically engineered variant BrCas12b of the present disclosure described above, such as a variant of Brevibacillus sp. SYP-B805 Cas12b having at least one mutation selected from: K44W, S92L, F208W, N524V, D567W, R634L, L795I, D868I, D868V, T874H, T874S, D951F, A1015E, R159E, D209R, F208W, and combinations thereof.

In some embodiments the target polynucleotide can be a DNA or RNA associated with a specific condition/disease, such as cancer, genetic disease, or infectious agent (e.g., bacteria, virus, fungal, etc.). In embodiments, the target polynucleotide is a virus, such as, but not limited to SARS-CoV-2, HIV, HCV, Chagas, malaria, and the like. Thus, in embodiments the crRNA can be complimentary to and configured to bind a complimentary region on the target DNA or RNA of interest to detect the specific condition/disease, such as cancer, genetic disease, infection agent (e.g., bacteria, virus, etc.) and the like. According to the methods of the present disclosure, when the sample is combined with all of these elements in the reaction vessel at the elevated temperatures, the target polynucleotide in the sample is amplified by the isothermal amplification components, and then the CrRNA binds the amplified target polynucleotides and forms a CRISPR/Cas complex with the thermostable BrCAS12b to activate the trans cleavage activity of the BrCas12b, which cleaves probes in the reaction vessel to create a detectable signal and indicate the presence of the target polynucleotide in the sample, thereby diagnosing the specific condition/disease associated with the target polynucleotide. Thus, in embodiments, the methods of the present disclosure can include diagnosing a condition associated with the target polynucleotide upon detection of the detectable signal.

In embodiments the target polynucleotide is from a virus such as, but not limited to, SARS-CoV-2, HIV, HCV, Chagas, malaria, etc., and the CRSPR/BrCas12b complex binds the target polynucleotide, activating trans cleavage activity, cleaving the probes, and detecting the presence of the virus in the sample. In some embodiments the target polynucleotide is a specific for a variant of a virus, such as variants of SARS-CoV-2, and the specificity of the crRNA is specific enough to distinguish between variants of SARS-CoV-2, such as, but not limited to Alpha (B.1.1.7), Beta (B.1.352), Delta (B.1.617.2), and Gamma (P1 wherein the set of isothermal amplification components comprise loop-mediated isothermal amplification (LAMP) or reverse-transcription LAMP (RT-LAMP) enzymes and primers. Embodiments of sequences for isothermal amplification primers for SARS-CoV-2 are described in the tables and examples below.

In some embodiments, the set of isothermal amplification components added to the reaction vessel also includes an isothermal amplification reporter configured to produce an amplification-generated detectable signal or detectable molecule upon amplification of the target polynucleotide, wherein the amplification-generated detectable signal or detectable molecule of the isothermal amplification reporter is different and distinguishable from the CRISPR-generated detectable signal or detectable molecule produced by cleavage of the probes. In some embodiments, the isothermal amplification primers (e.g, LAMP primers) are not as specific for different variants of a target polynucleotide (e.g., virus variants) and can amplify different variants of the same target (such as, different variants of a virus, including, for instance different variants of SARS-CoV-2). Thus, in embodiments, the isothermal amplification reporter can detect the presence of a target polynucleotide associated with a specific condition, but may not be able to discern between different variants. However, the crRNA can be designed to specifically bind to a specific variant, such that the crRNA/BrCas12b complex only binds and activates trans cleavage of the probes in the presence of the target polynucleotide from the specific variant. In embodiments, the detectable signal produced by the isothermal amplification reporter can indicate the presence of a target polynucleotide associated with a specific condition, and the CRISPR-generated detectable signal produced by cleavage of the probe indicates the presence of a specific variant of the condition. For instance, in an embodiment described in greater detail in the examples below, an isothermal amplification reporter can indicate the presence in the sample of SARS-CoV-2 (any variant), while cleavage of the probe by the CRISPR/Cas complex indicates the presence in the sample of a specific variant of SARS-CoV-2, such as but not limited to Alpha (B.1.1.7), Beta (B.1.352), Delta (B.1.617.2), and Gamma (P1). In embodiments the reaction vessel could contain different crRNAs each configured to specifically bind and identify a different variant of SARS-CoV-2, allowing for detection of and the target polynucleotide and determination of the variant. This method has the advantage of allowing rapid, on-site, simultaneous determination of a positive COVID case in a patient as well as identification of circulating variants. Embodiments of sequences of crRNA for distinguishing different variants of SARS-CoV-2 are described in the tables and examples below.

It has also been found that adding sucrose to the reaction increases the detection capability of Cas12b enzymes at elevated temperatures. Although the BrCas12b enzymes of the present disclosure (both wild type and genetically engineered thermostable variants) already have increased stability and/or activity at the elevated temperatures used in isothermal amplification, sucrose can further increase the detection capability, such as by increasing thermal stability and or melting temperature. Thus, in embodiments of methods and systems of the present disclosure, sucrose can be added to reaction vessel to increase detection capability of Cas12b enzymes of the present disclosure at elevated temperatures.

It has further been discovered that performing CRISPR-based detection at elevated temperatures, such as the methods of the present disclosure, makes the reaction less or non-PAM restrictive (at least in part due to the fact that at the elevated temperatures, dsDNA denatures for the isothermal amplification, and thus the PAM sequence, which is located on the non-target strand, is no longer associated with the target strand, and the crRNA recognizes and binds to the target strand without the associated PAM sequence. Thus, methods of the present disclosure also include PAM-less nucleic acid detection during one-pot isothermal amplification and CRISPR-based detection at temperatures from about 60-70° C., such as temperatures from about 62-68° C. or other intervening or overlapping ranges.

Additional description of embodiments of methods of the present disclosure are provided in the examples below. Other methods for use of the isothermal amplification/CRISPR/Cas systems of the present disclosure can be appraised by one of skill in the art.

### Kits

The present disclosure also includes kits including some or all of the system components described above to be combined with a sample and instructions for use. In embodiments, the kit is shelf-stable, such that it can be transported and stored at ambient temperatures until use. In embodiments, the contents of the kit are lyophilized, such that they are non-reactive and stable without the need for refrigeration until combined with a sample and incubated at elevated temperatures for isothermal amplification and CRISPR/Cas detection.

In embodiments, shelf-stable kits of the present disclosure for detecting a target polynucleotide in a sample in a single-pot including the following components:
a) a set of lyophilized isothermal amplification components including isothermal amplification enzymes and primers as described above that are configured to recognize and amplify the target polynucleotide and also are lyophilized for longer shelf life and ease of transport.
b) a lyophilized BrCas12b CRISPR-associated (Cas) enzyme, where the Cas enzyme is a BrCas12b enzyme of the present disclosure as described above where the Cas enzyme has been lyophilized for stability;
c) a lyophilized guide RNA sequence, where the guide RNA sequence includes a CRISPR RNA (crRNA) sequence of the present disclosure as described above configured to bind to the target polynucleotide and a tracrRNA sequence configured to interact with the BrCas12b Cas enzyme to form a CRISPR/Cas complex upon binding of the crRNA sequence to the target polynucleotide and where the crRNA sequence and the tracrRNA sequence are lyophilized;
d) a plurality of lyophilized probes, where the probes are probes of the present disclosure as described above each probe having an oligonucleotide element labeled with a detectable label, where the probe is configured to be cleaved by the BrCas12bCas enzyme when the crRNA sequence binds the target polynucleotide to generate a detectable signal or molecule and where the probes have been lyophilized; and
instructions for combining components a-d in a single reaction vessel with a sample, incubating the sample at a temperature of about 60-70° C. for a period of time, and detecting the detectable signal or molecule.

In embodiments, the kit also includes a lyophilized isothermal amplification buffer, such as a lyophilized version of NEB LAMP Master Mix. In embodiments, the lyophilized isothermal amplification buffer does not contain glycerol. Kits of the present disclosure can also include any of the elements described in the systems and methods above. In embodiments, additional elements can also be lyophilized to render them shelf-stable.

Additional details regarding the methods, systems, and kits of the present disclosure are provided in the Examples below. The specific examples below are to be construed as merely illustrative, and not limitative of the remainder of the disclosure in any way whatsoever. Without further elaboration, it is believed that one skilled in the art can, based on the description herein, utilize the present disclosure to its fullest extent.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and protected by the following claims.

### Various Aspects and Embodiments of the Present Disclosure

The present disclosure further includes the following aspects and embodiments.

Aspect 1: A genetically engineered variant BrCas12b CRISPR-associated (Cas) enzyme comprising a peptide sequence of SEQ ID NO: 1 with at least one mutation from the group consisting of: K44W, S92L, F208W, N524V, D567W, R634L, L795I, D868I, D868V, T874H, T874S, D951F, A1015E, R159E, D209R, F208W, and combinations thereof.

Aspect 2: The genetically engineered variant BrCas12B enzyme of aspect 1, wherein the genetically engineered variant BrCas12B enzyme has greater thermal stability, greater nuclease activity, or both, as compared to a corresponding wild type BrCas12b Cas enzyme.

Aspect 3: The genetically engineered variant BrCas12B enzyme of aspect 1 or 2, wherein the genetically engineered variant BrCas12B enzyme retains structural stability and has enzymatic activity at temperatures from about 55-70° C.

Aspect 4: A method of detecting a target polynucleotide in a sample, the method comprising:
combining the sample in a single reaction vessel comprising:
   a set of isothermal amplification components comprising isothermal amplification enzymes and primers configured to recognize and amplify the target polynucleotide;
   a BrCas12b CRISPR-associated (Cas) enzyme;
   an sgRNA sequence comprising a CRISPR RNA (crRNA) sequence configured to bind to the target polynucleotide and a tracrRNA sequence configured to interact with the BrCas12b Cas enzyme to form a CRISPR/Cas complex upon binding of the crRNA sequence to the target polynucleotide; and
a plurality of probes, each probe comprising an oligonucleotide element labeled with a detectable label, wherein the probe is configured to be cleaved by the BrCas12b Cas enzyme when the crRNA sequence binds the target polynucleotide to generate a CRISPR-generated detectable signal or detectable molecule;
incubating the contents of the reaction vessel at a temperature of about 60-70° C. for a period of time; and
detecting the CRISPR-generated detectable signal or detectable molecule if the target polynucleotide is present in the sample.

Aspect 5: The method of aspect 4, further comprising adding to the reaction vessel an isothermal amplification buffer compatible with the isothermal amplification components and the BrCas12b Cas enzyme.

Aspect 6: The method of aspect 4 or 5, wherein the BrCas12b Cas enzyme is Brevibacillus sp. SYP-B805 Cas12b having a peptide sequence of SEQ ID NO: 1 or a genetically engineered variant thereof.

Aspect 7: The method of aspect 6, wherein the BrCas12b Cas enzyme is a genetically engineered variant of Brevibacillus sp. SYP-B805 Cas12b having a higher thermostability than the wild type Brevibacillus sp. SYP-B805 Cas12b.

Aspect 8: The method of aspect 7, wherein the genetically engineered variant of Brevibacillus sp. SYP-B805 comprises at least one mutation from the group consisting of: K44W, S92L, F208W, N524V, D567W, R634L, L795I, D868I, D868V, T874H, T874S, D951F, A1015E, R159E, D209R, F208W, and combinations thereof.

Aspect 9: The method of any of aspects 4-8, wherein the set of isothermal amplification components further comprises an isothermal amplification reporter configured to produce an amplification-generated detectable signal or detectable molecule upon amplification of the target polynucleotide, wherein the amplification-generated detectable signal or detectable molecule of the isothermal amplification reporter is different and distinguishable from the CRISPR-generated detectable signal or detectable molecule produced by cleavage of the probes.

Aspect 10: The method of aspect 9, wherein the isothermal amplification reporter is an SYTO dye.

Aspect 11: The method of any of aspects 4-9, wherein the probe is selected from a FAM-polyT-Quencher (PAM-FQ) and a HEX-polyT-Quencher (HEX-FQ) reporter.

Aspect 12: The method of any of aspects 4-11, wherein the oligonucleotide element of the probe is a ssDNA and is about 80% of A and/or T.

Aspect 13: The method of any of aspects 4-12, wherein the oligonucleotide element of the probe comprises a nucleotide sequence selected from TTATT (SEQ ID NO: 3) and TTTTTTTT (SEQ ID NO: 4).

Aspect 14: The method of any of aspects 4-13, wherein incubating comprises incubating the contents of the reaction vessel at a temperature of about 62-68° C.

Aspect 15: The method of any of aspects 4-14, wherein the contents of the reaction vessel are incubated for about 8-30 minutes.

Aspect 16: The method of any of aspects 4-15, wherein the set of isothermal amplification components comprise loop-mediated isothermal amplification (LAMP) or reverse-transcription LAMP (RT-LAMP) enzymes and primers.

Aspect 17: The method of aspect 16, wherein the LAMP/RT-LAMP enzymes comprise a thermostable polymerase and wherein the LAMP/RT-LAMP primers comprise at least a forward internal primer (FIP), a backward internal primer (BIP), a forward outer primer, and a backward outer primer.

Aspect 18: The method of aspect 17, wherein the target polynucleotide is DNA and the set of isothermal amplification components comprise LAMP enzymes and primers and a thermostable polymerase.

Aspect 19: The method of aspect 17, wherein the target polynucleotide is RNA and the set of isothermal amplification components comprise RT-LAMP enzymes and primers and wherein the RT-LAMP enzymes comprise a thermostable reverse transcriptase and thermostable a polymerase.

Aspect 20: The method of any of aspects 4-19, wherein the target polynucleotide is a SARS-CoV-2 polynucleotide.

Aspect 21: The method of aspect 20, wherein the method can distinguish between variants of SARS-CoV-2.

Aspect 22: The method of aspect 21, wherein the variants are selected from Alpha (B.1.1.7), Beta (B.1.352), Delta (B.1.617.2), Gamma (P1).

Aspect 23: The method of aspect 22, wherein the target polynucleotide is a SARS-COV-2 variant selected from Alpha (B.1.1.7), Beta (B.1.352), Delta (B.1.617.2), and Gamma (P1), the method further comprising including in the reaction vessel an isothermal amplification reporter configured to produce an amplification-generated detectable signal or molecule upon amplification of the SARS-CoV-2 polynucleotide, wherein the amplification-generated detectable signal or molecule of the isothermal amplification reporter is different and distinguishable from the CRISPR-generated detectable signal or molecule produced by cleavage of the probes, wherein detecting the amplification-generated detectable signal or molecule indicates the presence of SARS-CoV-2 in the sample, and detecting the CRISPR-generated detectable signal or molecule indicates the presence of the SARS-CoV-2 variant.

Aspect 24: The method of any of aspects 4-23, further comprising adding sucrose to the reaction vessel at a concentration of about 100 mM-350 mM, wherein the sucrose increases the detection capability of the BrCas12b enzyme.

Aspect 25: A one-pot nucleic acid detection system for detecting a target polynucleotide in a sample, the system comprising:
a set of isothermal amplification components comprising isothermal amplification enzymes and primers configured to recognize and amplify the target polynucleotide;
a BrCas12b CRISPR-associated (Cas) enzyme;
an sgRNA sequence comprising a CRISPR RNA (crRNA) sequence configured to bind to the target polynucleotide and a tracrRNA sequence configured to interact with the BrCas12b Cas enzyme to form a CRISPR/Cas complex upon binding of the crRNA sequence to the target polynucleotide; and
a plurality of probes, each probe comprising an oligonucleotide element labeled with a detectable label, wherein the probe is configured to be cleaved by the BrCas12bCas enzyme when the guide sequence binds the target polynucleotide to generate a CRISPR-generated detectable signal or molecule.

Aspect 26: The system of aspect 25, further comprising a single reaction vessel configured to contain the elements of the system of aspect X in a single pot and further comprising a heating element to maintain the reaction vessel at a temperature of about 60-70° C.

Aspect 27: The system of aspect 25 or 26, further comprising an isothermal amplification buffer compatible with the isothermal amplification components and the BrCas12b Cas enzyme.

Aspect 28: The system of any of aspects 25-27, wherein the BrCas12b is Brevibacillus sp. SYP-B805 Cas12b having the polypeptide sequence SEQ ID NO: 1 or a genetically engineered variant thereof.

Aspect 29: The system of aspect 28, wherein the BrCas12b Cas enzyme is a genetically engineered variant of Brevibacillus sp. SYP-B805 Cas12b having a higher thermostability than the wild type Brevibacillus sp. SYP-B805 Cas12b.

Aspect 30: The system of aspect 29, wherein the genetically engineered variant of Brevibacillus sp. SYP-B805 comprises at least one mutation from the group consisting of: K44W, S92L, F208W, N524V, D567W, R634L, L795I, D868I, D868V, T874H, T874S, D951F, A1015E, R159E, D209R, F208W, and combinations thereof.

Aspect 31: The system of any of aspects 25-30, wherein the set of isothermal amplification components further comprises an isothermal amplification reporter configured to produce an amplification-generated detectable signal or detectable molecule upon amplification of the target polynucleotide, wherein the amplification-generated detectable signal or detectable molecule of the isothermal amplification reporter is different and distinguishable from the CRISPR-generated detectable signal or detectable molecule produced by cleavage of the probes.

Aspect 32: The system of aspect 31, wherein the isothermal amplification reporter is SYTO9 dye and the probe is a HEX-FQ reporter.

Aspect 33: The system of aspect 32, wherein the oligonucleotide element of the probe comprises a nucleotide sequence selected from TTATT and TTTTTTTT.

Aspect 34: The system of any of aspects 25-33, wherein the set of isothermal amplification components comprise loop-mediated isothermal amplification (LAMP) or reverse-transcription LAMP (RT-LAMP) enzymes and primers.

Aspect 35: The system of aspect 34, wherein the LAMP/RT-LAMP enzymes comprise a thermostable polymerase and wherein the LAMP/RT-LAMP primers comprise at least a forward internal primer (FIP), a backward internal primer (BIP), a forward outer primer, and a backward outer primer.

Aspect 36: The system of aspect 35, wherein the target polynucleotide is RNA and the set of isothermal amplification components comprise RT-LAMP enzymes and primers and wherein the RT-LAMP enzymes comprise a thermostable reverse transcriptase and thermostable a polymerase.

Aspect 37: The system of any of aspects 25-36, wherein the target polynucleotide is a SARS-CoV-2 polynucleotide.

Aspect 38: The system of aspect 37, wherein the method can distinguish between variants of SARS-CoV-2.

Aspect 39: The system of aspect 38, wherein the target polynucleotide is a SARS-CoV-2 variants are selected from Alpha (B.1.1.7), Beta (B.1.352), Delta (B.1.617.2), and Gamma (P1), the method further comprising including in the reaction vessel an isothermal amplification reporter configured to produce an amplification-generated detectable signal or molecule upon amplification of the SARS-CoV-2 polynucleotide, wherein the amplification-generated detectable signal or molecule of the isothermal amplification reporter is different and distinguishable from the CRISPR-generated detectable signal or molecule produced by cleavage of the probes, wherein detecting the amplification-generated detectable signal or molecule indicates the presence of SARS-CoV-2 in the sample, and detecting the CRISPR-generated detectable signal or molecule indicates the presence of the SARS-CoV-2 variant.

Aspect 40: A shelf-stable kit for detecting a target polynucleotide in a sample comprising the following components:
a) a set of lyophilized isothermal amplification components comprising isothermal amplification enzymes and primers configured to recognize and amplify the target polynucleotide;
b) a lyophilized BrCas12b CRISPR-associated (Cas) enzyme;
c) a lyophilized sgRNA sequence comprising a CRISPR RNA (crRNA) sequence configured to bind to the target polynucleotide and a tracrRNA sequence configured to interact with the BrCas12b Cas enzyme to form a CRISPR/Cas complex upon binding of the crRNA sequence to the target polynucleotide;
d) a plurality of lyophilized probes, each probe comprising an oligonucleotide element labeled with a detectable label, wherein the probe is configured to be cleaved by the BrCas12bCas enzyme when the crRNA sequence binds the target polynucleotide to generate a CRISPR-generated detectable signal or molecule; and
instructions for combining components a-d with a sample, incubating the sample at a temperature of about 60-70° C. for a period of time, and detecting the detectable signal or molecule.

Aspect 41: The kit of aspect 40, wherein the kit further comprises lyophilized isothermal amplification buffer compatible with the isothermal amplification components and the BrCas12b Cas enzyme.

Aspect 42: The kit of aspect 40 or 41, wherein the BrCas12b is Brevibacillus sp. SYP-B805 Cas12b having the polypeptide sequence SEQ ID NO: 1 or a genetically engineered variant thereof.

The following examples are put forth so as to provide those of ordinary skill in the art with a complete disclosure and description of how to perform the methods and use the compositions and compounds disclosed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value. In addition, the phrase “about ‘x’ to ‘y’” includes “about ‘x’ to about ‘y’”.

### Sequences:

The following list and tables provide sequences of nucleic acids and polypeptides described in the present disclosure and/or used in the examples.

polypeptide sequence of wild type Brevibacillus sp.
SYP-B805 Cas12b
SEQ ID NO: 1
MPVRSFKVKLVTRSGDAEHMLQLRRGLWKTHEIVNQGIAYYMNKL
ALMRQEPYAGKSREVVRLELLHSLRAQQKRNNWTGDAGTDDEILN
LSRRLYELLVPSAIGEKGDAQMLSRKFLSPLVDPNSEGGKGTAKS
GRKPRWMKMREEGHPDWEAEREKDRAKKAADPTASILNDLEAFGL
RPLFPLFTDEQKGIQWLPKQKRQFVRTFDRDMFQQALERMLSWES
WNRRVAEEYQKLQAQRDELYAKYLADGGAWLEALQSFEKQREVEL
AEESFAAKSEYLITRRQIRGWKQVYEKWSQLPEHAAQEQFWQVVA
DVQTSLPGAFGDPKVYQFLSQPEHHHIWRGYPNRLFHYSDYNGVR
KKLQRARHDATFTLPDPVEHPLWIRFDARGGNIHDYEISQNGKQY
QVTFSRLLWPENETWVERENVTVAIGASQQLKRQIRLDGYADKKQ
KVRYRDYSSGIELTGVLGGAKIQFDRRHLRKASNRLADGETGPVY
LNVVVDIEPFLAMRNGRLQTPIGQVLQVNTKDWPKVTGYKPAELI
SWIQNSPLAVGTGVNTIEAGMRVMSVDLGQRSAAAVSIFEVMRQK
PAEQETKLFYPIAVTGLYAVHRRSLLLRLPGEKISDEIEQQRKIR
AHARSLVRYQIRLLADVLRLHTRGTAEQRRAKLDELLATLQTKQE
LDQKLWQTELEKLFDYIHEPAERWQQALVAAHRTLEPVIGQAVRH
WRKSLRIDRKGLAGMSMWNIEELEETRKLLIAWSKHSRVPGEPNR
LDKEETFAPQQLQHIQNVKDDRLKQMANLLVMTALGYKYDEAEKQ
WKEAYPACQMILFEDLSRYRFALDRPRRENNRLMKWAHRSIPRLV
YLQGELFGIQVGDVYSAYTSRFHAKTGAPGIRCHALKEEDLQPNS
YVVKQLIKDGFIREDQTGSLKPGQIVPWSGGELFVTLADRSGSRL
AVIHADINAAQNLQKRFWQQNTEIFRVPCKVTTSGLIPAYDKMKK
LFGKGYFAKINQTDTSEVYVWEHSAKMKGKTTPADPAEEGVFDES
LTDEMEELEDSQEGYKTLFRDPSGFFWSSDRWLPQKEFWFWVKRR
IEKKLREQLQ
E. coli codon optimized nucleotide sequence
encoding wild type Brevibacillus sp.
SYP-B805 Cas12b
SEQ ID NO: 2
atgcccgtaaggtcatttaaagttaagctagtgactcggtctggc
gatgcggagcacatgctgcaactgcgtagggggctgtggaaaacc
catgaaatcgtaaatcagggcattgcgtactacatgaacaagttg
gcgctgatgcgtcaagaaccgtatgctggtaaaagccgtgaggtt
gttcgtttggagctgctgcatagcctgcgtgcacagcagaaacgt
aataactggaccggcgacgccggtacggatgatgaaattctgaac
ctgagccgtcgcctgtacgagttgctcgtgccgagcgcgatcggc
gagaagggcgatgcgcagatgttatctcgcaagtttctgtcgccg
ctggttgatccgaattctgaaggtggtaaaggcacggcgaagtcc
ggccgtaagccgcgttggatgaaaatgcgggaggagggtcatccg
gattgggaagctgaacgcgagaaggaccgcgcaaagaaagcagcg
gacccgactgcgtctattctgaatgatttggaagcgtttggtctg
cgtccgctgttcccgctgttcaccgacgagcaaaaaggcatccag
tggctgccgaaacaaaaacgtcagttcgtgcgtaccttcgatcgt
gatatgtttcagcaggctctggagcgtatgctgtcctgggagtcc
tggaaccgccgtgtcgccgaggaatatcagaagctgcaggctcaa
cgtgacgagttgtacgcaaaatatctggccgatggtggtgcctgg
ctggaagcgttacaaagctttgagaagcagcgtgaagttgaactg
gctgaggagtcttttgccgctaaatccgaatatctgatcacccga
cgacaaattcgcggttggaaacaggtgtacgaaaagtggtcacaa
ctgccagagcacgccgcccaagaacaattctggcaagttgtagcg
gacgtacagacgtctctgcctggtgcgtttggtgacccaaaagtt
taccagtttctgagccagccggagcatcaccacatctggcgcggt
tacccgaaccgactattccactattctgattacaacggtgttcgc
aagaagctgcaaagagcgcgtcacgatgcgacttttaccttgccg
gacccggttgaacacccgctttggatcagattcgatgcgcgtggt
ggtaatattcatgattatgagatcagccagaatggcaagcaatat
caagttaccttctcccgcctcctgtggcctgaaaatgaaacctgg
gtggagcgtgaaaacgttaccgttgcaatcggggcaagccagcag
cttaagcgtcagattcggttggacggctacgccgataaaaagcag
aaggtacgttatcgtgactactctagcggcatcgagctgaccggt
gttctgggtggtgcgaagatccagttcgaccgtcgtcacctgcgc
aaagcaagtaatcgtttagccgacggtgaaaccggccctgtgtac
ctgaacgtggtggtcgatatcgagccgtttctggctatgcgcaac
ggtcgtctacaaacgccgattggccaagtgctgcaagtcaacacc
aaggactggccgaaggtgacgggttataaaccggctgagctgatc
agctggattcaaaacagcccgctggcggtgggtacaggcgtcaac
accattgaggcgggtatgcgcgttatgagtgttgatctgggtcaa
agaagcgcagctgctgtgagcattttcgaggtcatgcgccagaaa
ccggcggaacaggaaaccaaactcttctatccgattgctgtgacc
ggtctgtatgcggtgcatcgtcgtagtttgctgctgcgtcttcca
ggcgagaaaattagcgacgaaatcgaacagcaacgtaaaatcaga
gcccatgctcgtagcttggtgcgctaccagatccgtctgcttgcg
gacgtactgcgtctccacacccgtggcaccgcggaacagagacgt
gcgaaactggatgaattgctggctaccctgcaaacgaaacaagag
ttagatcagaagttatggcaaactgagttggaaaagctgttcgac
tacattcacgaaccggcggagcgctggcagcaggcgctggtggcg
gcgcaccgtaccttggagccggttatcggtcaggcagttcgtcat
tggcgtaagtccctccgcattgatcgtaagggcttggcgggcatg
agcatgtggaacattgaagagttggaggaaacccggaagttgttg
atcgcatggagcaaacattccagggtccccggcgagccgaaccgt
ctggacaaagaagaaacgtttgcaccgcagcagctccaacatatc
caaaacgtcaaagacgaccgcttgaagcagatggcaaatctgctc
gtgatgaccgccttgggctacaagtacgatgaggccgagaaacaa
tggaaagaggcgtatccggcgtgtcagatgattctgttcgaagat
ctaagccgctaccgctttgcgctggaccgaccgcgtcgcgagaac
aaccggttaatgaaatgggcacacagaagtatcccgcgtctggtt
tatctgcaaggtgaattgttcggcatccaggtgggtgatgtttat
tcggcgtatactagccgtttccatgctaaaactggtgcgccaggc
attcgctgccacgcgctgaaagaagaagacctgcaaccgaacagc
tacgtggtgaagcaactgattaaagacggctttatccgtgaggac
cagaccggttcccttaagccgggtcagattgtaccgtggtctggc
ggtgaactgttcgttaccttagccgaccgttccggcagccgtttg
gccgtgatccacgccgacatcaatgcagcgcagaatctgcaaaag
agattctggcaacagaacaccgagatctttcgtgtgccgtgcaaa
gtgactacgtccggtctgataccggcatacgacaagatgaaaaag
ttattcggcaaaggctatttcgcaaagatcaaccagacggacacc
agcgaagtgtacgtttgggaacacagcgctaagatgaagggcaaa
acgaccccggcggacccagcagaagaaggagtttttgatgagtcc
ctgaccgacgagatggaagagcttgaagacagccaagagggttac
aagaccctgttccgtgatccgtcgggctttttttggtcgagcgat
cgctggctgccgcagaaagagttctggttttgggttaaacgccgt
atcgagaagaaactgcgcgagcagctccag

### Example RT-LAMP Primer Sequences

| | | | 1X |
|---|---|---|---|
| Primer | | | Concen- |
| target | Primer name/SEQ ID | Sequence | tration |
| N gene | N_F3/SEQ ID NO: 3 | AACACAAGCTTTCGGCAG | 0.2 μM |
| (for | N_B3/SEQ ID NO: 4 | GAAATTTGGATCTTTGTCATCC | 0.2 μM |
| presence | N_FIP/SEQ ID NO: 5 | TGCGGCCAATGTTTGTAATCAGCC | 1.6 μM |
| of | | AAGGAA ATTTTGGGGAC | |
| SARS- | N_BIP/SEQ ID NO: 6 | CGCATTGGCATGGAAGTCACTTTG | 1.6 μM |
| CoV-2) | | ATGGC ACCTGTGTAG | |
| | N_LF/SEQ ID NO: 7 | TTCCTTGTCTGATTAGTTC | 0.8 μM |
| | N_LB/SEQ ID NO: 8 | ACCTTCGGGAACGTGGTT | 0.8 μM |
| Alpha | Alpha_F3/SEQ ID NO: 9 | ATACACTAATTCTTTCACACGT | 0.2 μM |
| (B.1.1.7) | Alpha_B3/SEQ ID NO: 10 | CCTCTTATTATGTTAGACTTCTCAG | 0.2 μM |
| | Alpha_FIP/SEQ ID NO: 11 | GGAAAAGAAAGGTAAGAACAACCC | 1.6 μM |
| | | TGACAAAGTTTTCAGAT | |
| | Alpha_BIP/SEQ ID NO: 12 | ATGGTACTAAGAGGTTTGATTGGA | 1.6 μM |
| | | AGCAAAATAAACACCATC | |
| | Alpha_LF/SEQ ID NO: 13 | CCTGAGTTGAATGTAAAACTGAGG | 0.8 μM |
| | Alpha_LB/SEQ ID NO: 14 | AACCCTGTCCTACCATTTAA | 0.8 μM |
| Beta | Beta_F3/SEQ ID NO: 15 | ACACGCCTATTAATTTAGTGC | 0.4 μM |
| (B.1.352) | Beta_B3/SEQ ID NO: 16 | TAGAAAAGTCCTAGGTTGAAGA | 0.4 μM |
| | Beta_FIP/SEQ ID NO: 17 | CCTAGTGATGTTAATACCTATTGGC | 1.6 μM |
| | | TCCCTCAGGGTTTTTCG | |
| | Beta_BIP/SEQ ID NO: 18 | AAGTTATTTGACTCCTGGTCATAAT | 1.6 μM |
| | | AAGCTGCAGCACC | |
| | Beta_LF/SEQ ID NO: 19 | CAAATCTACCAATGGTTCTAAAGC | 0.8 μM |
| | Beta_LB/SEQ ID NO: 20 | GATTCTTCTTCAGGTTGGACAGC | 0.8 μM |
| Delta | Delta_F3/SEQ ID NO: 21 | CCCTACTTATTGTTAATAACGCT | 0.4 μM |
| (B.1.617.2) | Delta_B3/SEQ ID NO: 22 | ATTCTTAAACACAAATTCCCTAAG | 0.4 μM |
| | Delta_FIP/SEQ ID NO: 23 | TTTTTGTGGTAATAAACACCCAAAA | 1.6 μM |
| | | ACTAATGTTGTTATTAAAGTCTGTG | |
| | Delta_BIP/SEQ ID NO: 24 | CTAGTGCGAATAATTGCACTTTTGA | 1.6 μM |
| | | TGTTTTCCTTCAAGGTCCAT | |
| | Delta_LF/SEQ ID NO: 25 | ATGGATCATTACAAAATTGAAA | 0.8 μM |
| | Delta_LB/SEQ ID NO: 26 | ATATGTCTCTCAGCCTTTTCTT | 0.8 μM |
| Gamma | Gamma_F3/SEQ ID NO: 27 | CAATTAATTGCCAGGAACCTAA | 0.2 μM |
| (P1) | Gamma_B3/SEQ ID NO: 28 | TACTGCCAGTTGAATCTGA | 0.2 μM |
| | Gamma_FIP/SEQ ID NO: 29 | CAACACGAACGTCATGATACTCTA | 1.6 μM |
| | | AGGGTAGTCTTGTAGTG | |
| | Gamma_BIP/SEQ ID NO: 30 | ACTAAAATGTCTGATAATGGACCC | 1.6 μM |
| | | CGGGTCCACCAAACGTAAT | |
| | Gamma_LF/SEQ ID NO: 31 | AAAGTCTTCATAGAACGAACAA | 0.8 μM |
| | Gamma_LB/SEQ ID NO: 32 | AAAATCAGCGAAATGCACCCC | 0.8 μM |
| RNase | RNaseP_F3/SEQ ID NO: 33 | TTGATGAGCTGGAGCCA | 0.2 μM |
| P | RNaseP_B3/SEQ ID NO: 34 | CACCCTCAATGCAGAGTC | 0.2 μM |
| | RNaseP_FIP/SEQ ID NO: 35 | GTGTGACCCTGAAGACTCGGTTTT | 1.6 μM |
| | | AGCCACTGACTCGGATC | |
| | RNaseP_BIP/SEQ ID NO: 36 | CCTCCGTGATATGGCTCTTCGTTTT | 1.6 μM |
| | | TTTCTTACATGGCTCTGGTC | |
| | RNaseP_LF/SEQ ID NO: 37 | ATGTGGATGGCTGAGTTGTT | 0.8 μM |
| | RNaseP_LB/SEQ ID NO: 38 | CATGCTGAGTACTGGACCTC | 0.8 μM |

### Example Single-Guide RNA Sequences

| Target | SEQ ID | Sequence |
|---|---|---|
| N gene (for | SEQ ID NO: 39 | GAAGGUGGUUAGCUACAGGCUGACCAGUGCA |
| presence of | | GUUGUGUCAUGUGCUACGGUGACCUAACACG |
| SARS-CoV-2) | | UCACUCAGUCACAACGGCUAUCUAUAUUUCCA |
| | | CUAACCAAAGUUAGUGGAAAUGUAGAUGGUUA |
| | | GCACCGAAGAACGCUGAAGCGCUG |
| Alpha (B.1.1.7) | SEQ ID NO: 40 | GAAGGUGGUUAGCUACAGGCUGACCAGUGCA |
| | | GUUGUGUCAUGUGCUACGGUGACCUAACACG |
| | | UCACUCAGUCACAACGGCUAUCUAUAUUUCCA |
| | | CUAACCAAAGUUAGUGGAAAUGUAGAUGGUUA |
| | | GCACGUUCCAUGCUAUCUCUGGGA |
| Beta (B.1.352) | SEQ ID NO: 41 | GAAGGUGGUUAGCUACAGGCUGACCAGUGCA |
| | | GUUGUGUCAUGUGCUACGGUGACCUAACACG |
| | | UCACUCAGUCACAACGGCUAUCUAUAUUUCCA |
| | | CUAACCAAAGUUAGUGGAAAUGUAGAUGGUUA |
| | | GCACUAUGUAAAGUUUGAAACCUA |
| Delta (B.1.617.2) | SEQ ID NO: 42 | GAAGGUGGUUAGCUACAGGCUGACCAGUGCA |
| | | GUUGUGUCAUGUGCUACGGUGACCUAACACG |
| | | UCACUCAGUCACAACGGCUAUCUAUAUUUCCA |
| | | CUAACCAAAGUUAGUGGAAAUGUAGAUGGUUA |
| | | GCACGAUGGAAAGUGGAGUUUAUU |
| Gamma (P1) | SEQ ID NO: 43 | GAAGGUGGUUAGCUACAGGCUGACCAGUGCA |
| | | GUUGUGUCAUGUGCUACGGUGACCUAACACG |
| | | UCACUCAGUCACAACGGCUAUCUAUAUUUCCA |
| | | CUAACCAAAGUUAGUGGAAAUGUAGAUGGUUA |
| | | GCACGUUUGUUUGUUCGUUUAGAU |
| RNase P | SEQ ID NO: 44 | GAAGGUGGUUAGCUACAGGCUGACCAGUGCA |
| | | GUUGUGUCAUGUGCUACGGUGACCUAACACG |
| | | UCACUCAGUCACAACGGCUAUCUAUAUUUCCA |
| | | CUAACCAAAGUUAGUGGAAAUGUAGAUGGUUA |
| | | GCACAAUUACUUGGGUGUGACCCU |

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1—Thermostable Cas12b from Brevibacillus for Rapid, Sensitive, and Highly Specific One-Pot Detection of SARS-CoV-2 Variants of Concern

The present example describes a complete one-pot detection reaction using a thermostable Cas12b effector endonuclease from Brevibacillus sp. to overcome challenges detecting and discriminating SARS-CoV-2 VOCs in clinical samples. CRISPR-SPADE was then applied for discriminating SARS-CoV-2 VOCs, including Alpha (B.1.1.7), Beta (B.1.351), Gamma (P.1), Delta (B.1.617.2), and Omicron (B.1.1.529) and validated in 208 clinical samples. CRISPR-SPADE achieved 92.8% sensitivity, 99.4% specificity, and 96.7% accuracy within 10-30 minutes for discriminating the SARS-CoV-2 VOCs, in agreement with S gene sequencing, achieving a positive and negative predictive value of 99.1% and 95.1%, respectively. Interestingly, for samples with high viral load (Ct value ≤30), 100% accuracy and sensitivity were attained. To facilitate dissemination and global implementation of the assay, a lyophilised version of one-pot CRISPR-SPADE reagents was developed and combined with an in-house portable multiplexing device capable of interpreting two orthogonal fluorescence signals.

This technology enables real-time monitoring of RT-LAMP-mediated amplification and CRISPR-based reaction at a fraction of the cost of a qPCR system. The thermostable Brevibacillus sp. Cas12b offers relaxed primer design for accurately detecting SARS-CoV-2 VOCs in a simple and robust one-pot assay. The lyophilised reagents and simple instrumentation further enable rapid deployable point-of-care diagnostics that can be easily expanded beyond COVID-19.

### Introduction

Since the beginning of the COVID-19 pandemic, many strategies have been explored to develop rapid and sensitive detection kits to drive the diagnostics towards home-based testing(1-5). Current gold-standard Reverse Transcription-quantitative Polymerase Chain Reaction (RT-qPCR) tests require sophisticated instrumentation as well as intensive labour training, and therefore become a major hurdle for deployment in remote areas. CRISPR-based detection technologies hold promises for future rapid point-of-care diagnosis of infectious diseases and cancer(6, 7). This is in part due to their versatility in implementation and design that lie in the programmability of the guide RNA sequence. Taking advantage of the collateral cleavage property, many Cas effectors have been repurposed for nucleic acid detection such as Cas12a in DETECTR (DNA Endonuclease-Targeted CRISPR Trans Reporter)(5, 8) and in HOLMESv1 (a one-HOur Low-cost Multipurpose highly Efficient System) (9), Cas12b in HOLMESv2(10), and Cas13a in SHERLOCK (Specific High Sensitivity Enzymatic Reporter UnLOCKing)(11, 12). Many of these tests employ a two-pot assay in which a pre-amplification step such as RPA (reverse transcription recombinase polymerase amplification) or RT-LAMP (reverse transcription loop-mediated isothermal amplification) is required prior to CRISPR detection. This strategy increases reaction time and chances of carryover contamination.

Efforts into developing rapid amplification-free CRISPR tests have been made, such as FIND-IT (Fast Integrated Nuclease Detection In Tandem) that supplements Csm6 protein into a Cas13a reaction to trigger a cascade of amplified fluorescence signal. However, these tests rely on guide RNA targeting multiple regions of the SARS-CoV-2 RNA genome, which can be difficult to design to discriminate the variants of concerns (VOCs) due to the low number of mutations. Having the ability to differentiate the VOCs is still dependent on the enrichment of target viral genomes. STOPCovid (SHERLOCK testing in one pot) technology can potentially overcome these challenges by employing a thermostable Cas12b derived from Alicyclobacillus acidiphilus (AapCas12b) that can be combined with RT-LAMP into a one-pot detection assay. One major disadvantage of STOPCovid is that the wild-type AapCas12b collateral activity ceases to work above 60° C. and requires additional additives, such as taurine, and a longer incubation time to perform robustly due to suboptimal temperature conditions, leading to slower diagnostics. Additionally, the protein's performance at this temperature range restricts LAMP primer designs since RT-LAMP reactions are typically optimised at 60° C.-65° C.

### Methods

### Expression Plasmid Construction

BrCas12b gene sequence derived from Brevibacillus sp. SYP-B805 was obtained from the National Center for Biotechnology Information (GenBank ID: WP_165214399.1). The gene sequence was then codon-optimised using the GenSmart™ Codon Optimisation tool (Genscript) for bacterial protein expression (SEQ ID NO: 1), synthesised by Twist Bioscience, and cloned into PET28a⁺ vector.

### Protein Expression and Purification

The BrCas12b expression plasmid was transformed into BL21(DE3) competent E. coli cells. Individual colonies were picked and inoculated in 8 mL Luria Broth (Fisher Scientific) for 12-15 hours. The culture was added to a 2 L of homemade Terrific Broth for scale-up and shaken until the OD600 reached 0.5-0.8. The culture was then placed on ice for 15-30 minutes prior to the addition of 0.2 mM IPTG (Isopropyl β-d-1-thiogalactopyranoside) followed by overnight expression at 16° C. for 14-18 hours.

The next day, cells were harvested by centrifugation at 10,000×g for 5 minutes. Cell pellets were then resuspended in lysis buffer (0.5 M NaCl, 50 mM Tris-HCl, PH=7.5, 0.5 mM TCEP, 1 mM PMSF, 0.25 mg/ml lysozyme, and 10 μg/mL Deoxyribose nuclease I). The suspended cell mixture was disrupted by sonication and centrifuged at 39,800×g for 30 minutes at 4° C. The supernatant was collected and filtered through a 0.22 μm filter (Milipore Sigma) prior to injecting into a Histrap 5 mL FF column (Cytiva) pre-equilibrated with Wash Buffer (0.5 M NaCl, 50 mM Tris-HCl, PH=7.5, 0.5 mM TCEP, 20 mM Imidazole). The purification process was performed via an FPLC Biologic Duoflow system (Bio-rad). After lysate injection and washing with Wash Buffer A, the column was eluted with 40 mL of Elution Buffer B (0.5 M NaCl, 50 mM Tris-HCl PH=7.5, 0.5 mM TCEP, 300 mM Imidazole). The eluted fractions were pooled together, transferred to a 10 KDa-14 KDa dialysis bag, and dialysed in Dialysis Buffer (0.25 M NaCl, 40 mM HEPES, pH=7, 1 mM DTT) overnight at 4° C.

Following overnight dialysis, the BrCas12b mixture was concentrated down to 15 mL in a 30 kDa MWCO Vivaspin® 20 concentrator via centrifugation with the speed of 2000×g at 4° C. The concentrate was mixed at 1:1 ratio with Buffer C (150 mM NaCl, 50 mM HEPES, pH=7, 0.5 mM TCEP) before injecting into a Hitrap Heparin 1 mL HP column (Cytiva). The protein was eluted over a gradient buffer exchange from Buffer C to Buffer D (2 M NaCl, 50 mM HEPES, pH=7, 0.5 mM TCEP). Purest elution fractions of BrCas12b were collected, concentrated down, snap frozen, and stored at −80° C. until use. Experiment-ready BrCas12b was diluted in storage buffer (500 mM NaCl, 20 mM sodium acetate, 0.1 mM EDTA, 0.1 mM TCEP, 50% Glycerol, pH 7 @ 25° C.) which can be stored at −20° C.

AacCas12b and AapCas12b expression plasmids were obtained as a gift from Jennifer Doudna (Addgene plasmid #113433) and Wei Li (Addgene plasmid #121949), respectively. These two proteins were expressed and purified following Chen et al(8). and Teng et al(13).

### Nucleic Acid Preparation

For in vitro cleavage assay and differential scanning fluorimetry experiments, single-guide RNA (sgRNA) was synthesised using PCR (Takara) and in vitro transcription (IVT) using the HiScribe™ T7 Quick High Yield RNA Synthesis Kit (New England Biolabs). The IVT reaction was purified using the RNA Clean and Concentrator Kit (Zymo Research).

For patient sample detection, the sgRNA was either chemically synthesised by Integrated DNA Technologies (IDT) or enzymatically synthesised by an in vitro transcription (IVT) reaction followed by HPLC purification via the HPLC 1100 system (Agilent). The control genomic RNA for Beta, Delta, and Omicron variants were obtained from Twist Bioscience. The control genomic RNA for wild-type SARS-CoV-2 and Alpha were obtained from BEI Resources. Control genomic RNA for Gamma was obtained from Salemi Lab at the University of Florida.

In Vitro dsDNA Cleavage Assay

Cas12b, sgRNA and dsDNA were combined in 1× NEBuffer 2.1 on ice to a final concentration of 100 nM, 125 nM, and 7 nM, respectively. The reaction mixture was immediately transferred to a pre-set thermocycler and incubated for 1 hour at different temperatures (37° C., 47° C., 52° C., 57° C., 59° C., 61° C., 63° C., 65° C., 70° C., and 75° C.). After incubation, the reaction mixture was treated with 6× quenching buffer (30% glycerol, 1.2% SDS, 250 mM EDTA). The reaction products were analysed on 1% agarose gel pre-stained with GelRed (Biotium).

### Differential Scanning Fluorimetry

Cas12b and sgRNA were combined in a 1:2.5 ratio (1 μM:2.5 μM final concentration) in a mixture of Protein Thermal Shift™ buffer (Thermofisher) and 1× reaction buffer (100 mM NaCl, 50 nM Tris-HCl, pH=7.5, 1 mM DTT, and 10 mM MgCl₂). The reaction was incubated at 37° C. for 30 minutes to ensure complexation of sgRNA and Cas12b prior to adding the Protein Thermal Shift™ dye (Thermofisher). The reaction mixture was then transferred to qPCR StepOne Plus system (Thermofisher), and the binary complex temperature melting profile was recorded over a temperature range of 25-99° C. with a ramp rate of 1%/second. The experiment was carried in duplicates and repeated twice.

### Cas12b Trans-Cleavage Kinetic Assay

The trans-cleavage kinetic experiment was carried out following Cofsky et al (14), incorporated herein by reference, with modifications. In short, BrCas12b, sgRNA, and dsDNA activator were combined to a final concentration of 100 nM:125 nM:1 nM respectively in 1× NEBuffer 2.1 (New England Biolabs) and incubated at 62° C. for 30 minutes. HEX-polyT-Quencher reporter (FQ) at various concentrations (10 nM, 100 nM, 200 nM, 500 nM, 1 μM, and 2 μM) was added to the reaction mixture containing Cas12b trans activated complex, and the entire reaction was immediately transferred to a plate reader. Fluorescence measurements (λₑₓ: 483/30 nm, λₑₘ: 530/30 nm) were read every 30 seconds using the Biotek Synergy Neo (Agilent) that was pre-heated to 62° C. Initial velocity for each FQ concentration was determined by establishing the slope for all components and subtracting the slope for the no-activator control. The cleaved HEX-polyT reporter was titrated to different concentrations (10 nM, 100 nM, 200 nM, 500 nM, 1 μM, and 2 μM) and measured in the same experiment for the conversion of FQ fluorescence to concentration, eliminating non-linearity at high reporter concentrations.

### Patient Sample Collection

Saliva samples were collected and processed by following the guidelines approved by the University of Florida Institutional Review Board through Evaluating the Molecular Epidemiology of Coronavirus (COVID-19) in Florida (IRB202000633), Saliva Collection to Support Disease Diagnostics (IRB202002224), and Detecting SARS-CoV-2 RNA in human samples using an engineered CRISPR-based paper-based test (IRB202000781). Samples were extracted following CDC-recommended procedures and sequenced (described below) to identify SARS-CoV-2 lineages.

RNA extraction, library preparation and sequencing

Viral RNA was extracted from 180 μl of each saliva sample using the QIAamp 96 Viral RNA Kit with the QIAcube HT (Qiagen) using the following settings with a filter plate: the lysed sample was pre-mixed 8 times before subjecting to vacuum for 5 minutes at 25 kPa and vacuum for 3 min at 70 kPa. Following 3 washes using the same vacuum conditions above, the samples were eluted in 75 μl AVE buffer followed by a final vacuum for 6 minutes at 60 kPa. Next, nine microliters of RNA were used for cDNA synthesis and library preparation using the Illumina COVIDSeq Test kit (Illumina) and Mosquito HV Genomics Liquid Handler (SPT Labtech Inc.). The size and purity of the library were determined using the 4200 TapeStation System (Agilent) and the Qubit dsDNA HS Assay Kit (Life Technologies) according to the manufacturer's instructions. Constructed libraries were pooled and sequenced using the NovaSeq 6000 Sequencing System SP Reagent Kit and the NovaSeq Xp 2-Lane Kit. Illumina's DRAGEN pipeline was used to derive sample consensus sequences, which were filtered based on a minimum of 70% coverage of the genome.

### SARS-CoV-2 Viral Load Quantification

Levels of SARS-CoV-2 were determined using the 2019-nCoV_N1 assay (primer and probe set) with 2019-nCoV_N_positive control (IDT). Viral RNA was extracted as previously described then subjected to first-strand synthesis using ProtoScript II Reverse Transcriptase according to the manufacturer's instructions (New England Biolabs). Quantitative PCR was performed using TaqMan Fast Advanced Master Mix (ThermoFisher Scientific) according to the manufacturer's instructions. A standard curve was generated using N1 quantitative standards 10-fold diluted to determine viral copies. The assay was run in triplicate including one non-template control.

### One-Pot BrCas12b Detection Assay

LAMP primers targeting wild-type SARS-CoV-2 and variants of concern were designed using NEBR LAMP Primer Design Tool (New England Biolabs) and PrimerExplorer v5 at https://primerexplorer.jp/e/(15).

For the one-pot detection assay that tracks both target amplification and BrCas12b trans-cleavage, the reaction was assembled by combining the following reagents:

| Item | | Volume | Final |
|---|---|---|---|
| # | Reagents | (μL) | Concentration |
| 1 | WarmStart ® Multi-Purpose LAMP/ | 12.5 | 1X |
| | RT-LAMP 2X Master Mix (with UDG) | | |
| 2 | 10X LAMP Primer Mix | 2.5 | 1X |
| 3 | 100 μM HEX-polyT-Quencher Reporter | 0.5 | 2 μM |
| 4 | 25 μM SYTO ™ 9 Dye | 1 | 1 μM |
| 5 | 10 μM BrCas12b | 0.25 | 0.1 μM |
| 6 | 10 μM sgRNA | 0.5 | 0.2 μM |
| 7 | RNase-free Water | 2.75 | [symbol] |
| | Total | 20 | |

For the rapid one-pot detection assay monitoring BrCas12b trans-cleavage only, the reaction was assembled by combining the following reagents:

| Item | | Volume | Final |
|---|---|---|---|
| # | Reagents | (μL) | Concentration |
| 1 | WarmStart ® Multi-Purpose LAMP/ | 12.5 | 1X |
| | RT-LAMP 2X Master Mix (with UDG) | | |
| 2 | 10X LAMP Primer Mix | 2.5 | 1X |
| 3 | 100 μM HEX-polyT-Quencher Reporter | 0.5 | 2 μM |
| 4 | 10 μM BrCas12b | 0.25 | 0.1 μM |
| 5 | 10 μM sgRNA | 0.5 | 0.2 μM |
| 6 | RNase-free Water | 3.75 | [symbol] |
| | Total | 20 | |

For SARS-CoV-2 variant specificity testing, the one-pot reaction was added with LAMP primer and sgRNA corresponding to a SARS-CoV-2 variant and tested against other variants. For Limit of Detection (LoD) testing, RNA controls were subjected to serial dilution in triplicates and added to the one-pot reaction. For patient sample detection testing, 5 μL of extracted nucleic acid from clinical samples were added to the one-pot reaction.

The reaction mixture was transferred to a Bio-rad CFX96 Real-Time system with C1000 Thermal Cycler module. The reaction was isothermally incubated at 62° C. for all variant detection except Omicron, which was carried out at 60° C. Fluorescence measurements were read every 30 seconds per cycle over 120 cycles. The FAM channel (λₑₓ: 470/20 nm, λₑₘ: 520/10 nm) was used to monitor SYTO9™, and the HEX channel (λₑₓ: 525/10 nm, λₑₘ: 570/10 nm) was used to detect BrCas12b trans-cleavage via FRET-based HEX reporter.

### Lyophilization of BrCas12a One-Pot Detection Assay

Developmental lyo-ready Warmstart® Master Mix with Uracil-DNA glycosylases (UDG) (New England Biolabs) was combined with 100 nM BrCas12b, 200 nM sgRNA, and 1.25 μmoles Trehalose in ice. The mixture was placed in an aluminum cooling block and kept in dry ice for 30 minutes. The cooling block containing samples was then transferred to a freeze-drying system (Labconco) and lyophilized for 4 hours or overnight. To initiate the reaction, the lyophilized master mix was reconstituted in 20 μL RNase-free water.

### Imaging Using a Mobile Phone and a Lens

One-pot BrCas12b reagents were combined in an optically clear PCR tube (Applied Biosystems) followed by the addition of the extracted RNA sample and 62° C. incubation for 20 minutes. The reaction tube was then imaged using a battery-operated 410 nm-415 nm UVA-blue flashlight on a mobile phone in a dark setting. To visualise a sample containing FRET-based HEX reporter, the imaging system was assembled by attaching a combination of yellow and orange lenses onto the flashlight camera (NestEcho).

### Portable Multiplexing Detection Prototype (FISSH)

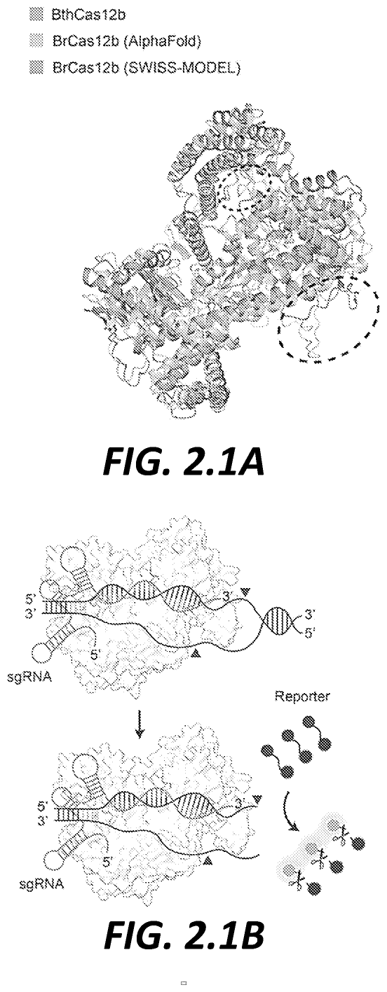

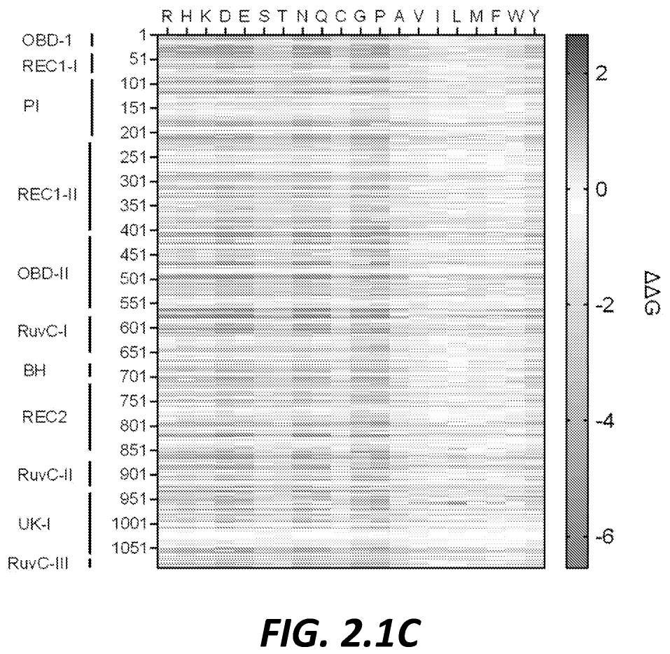

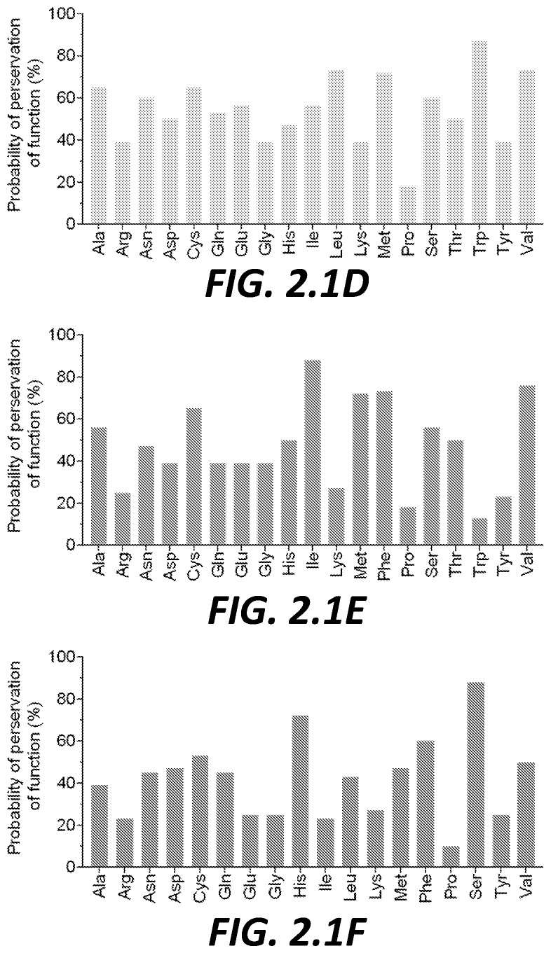

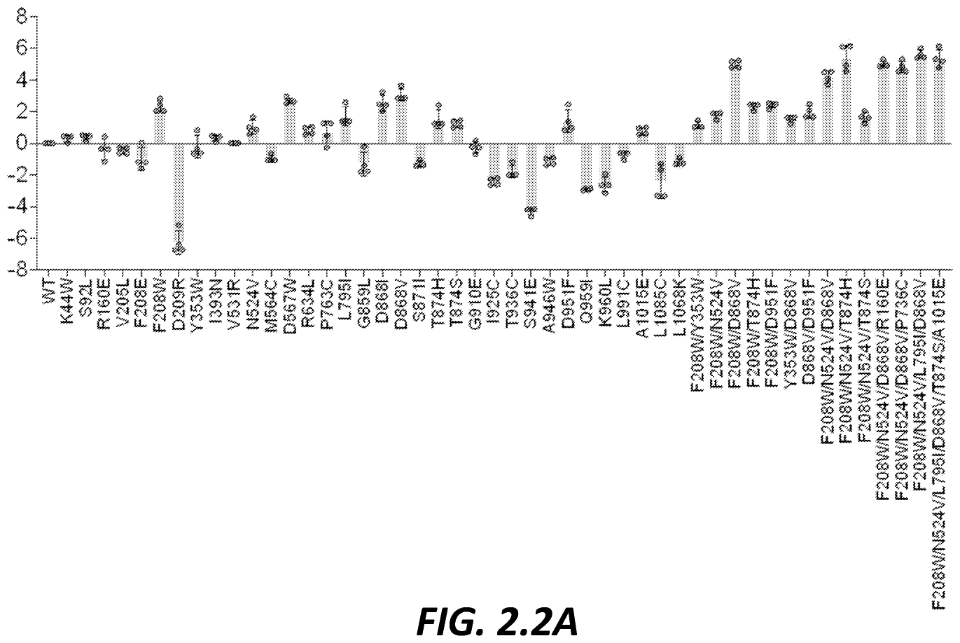

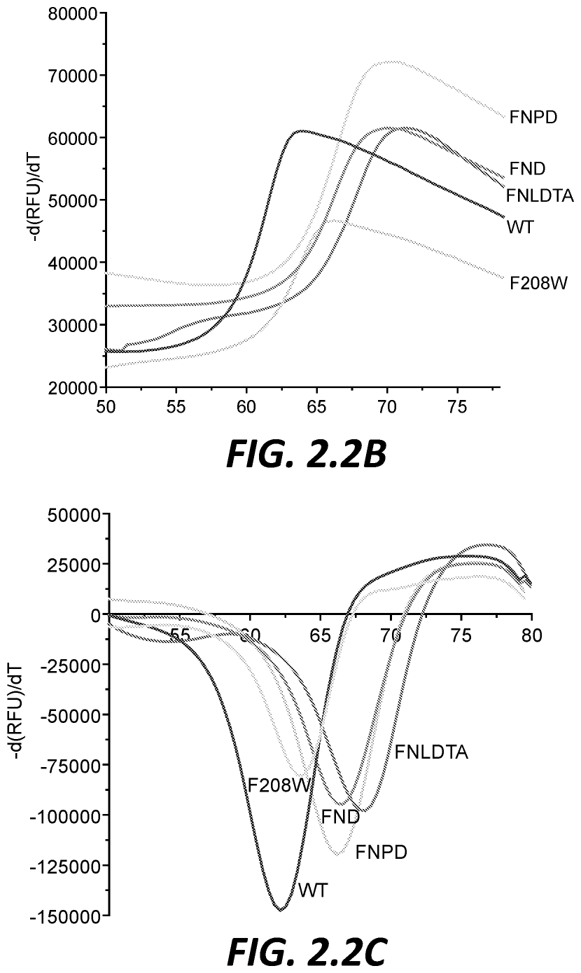

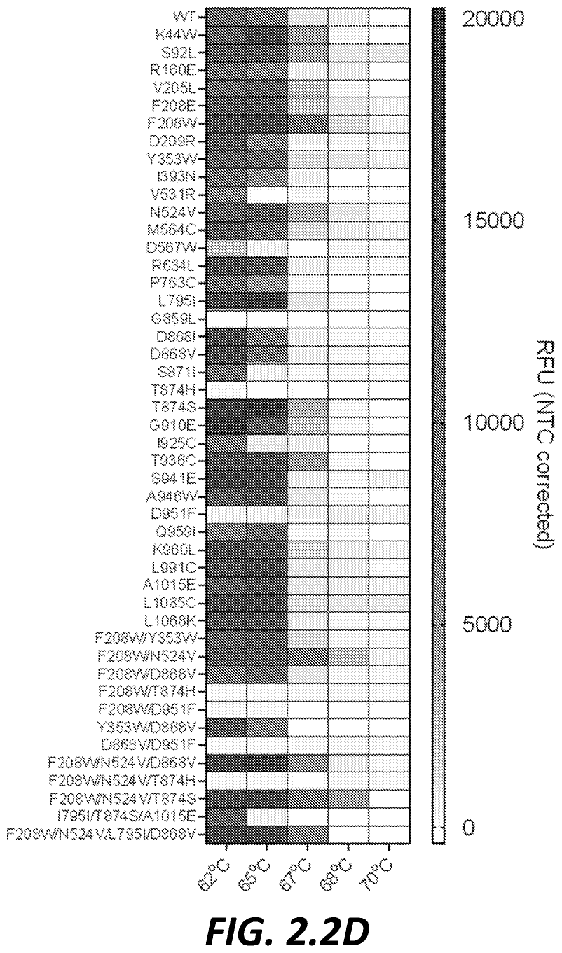

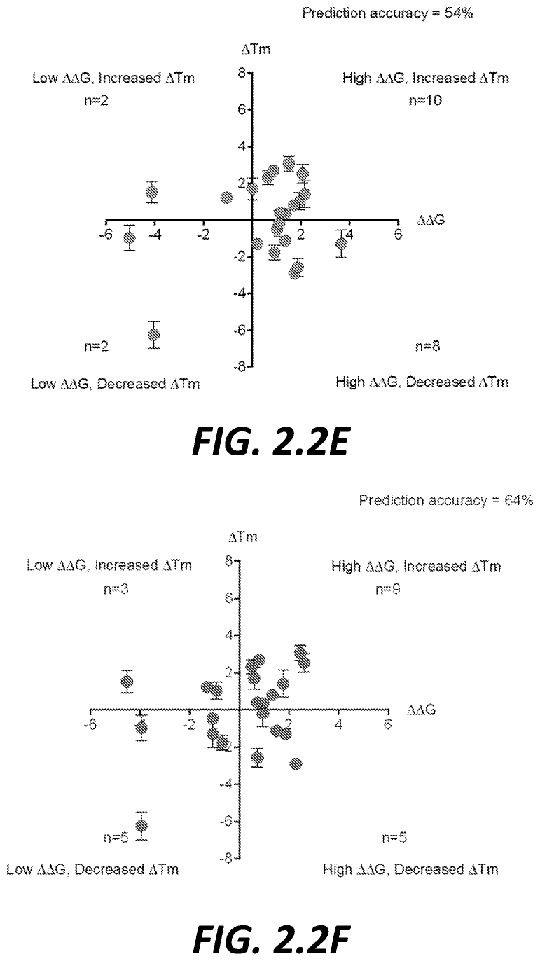

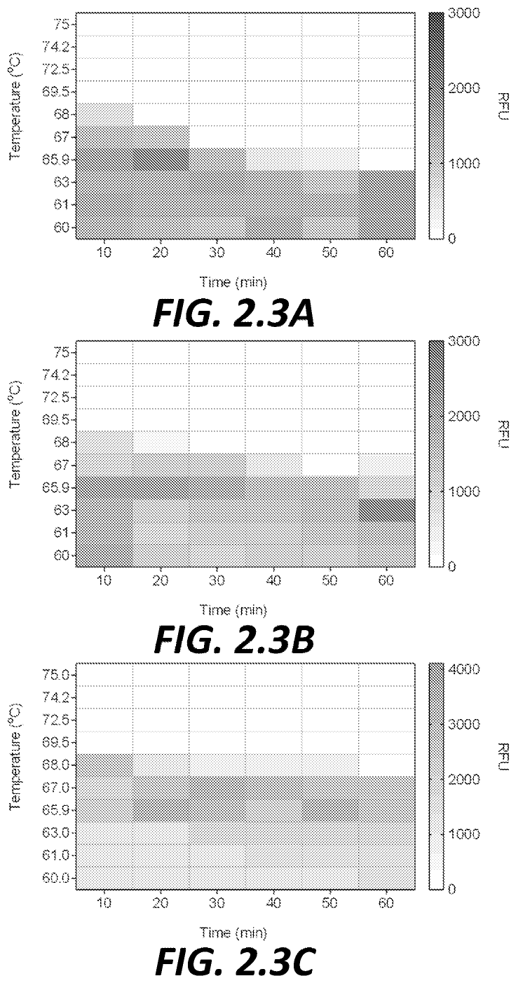

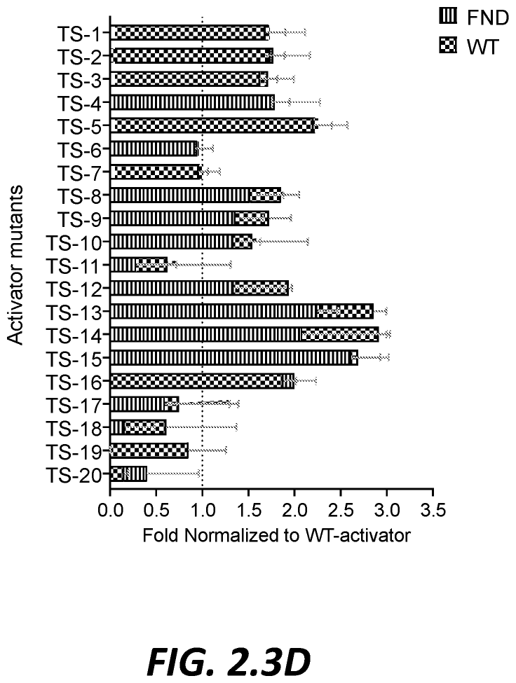

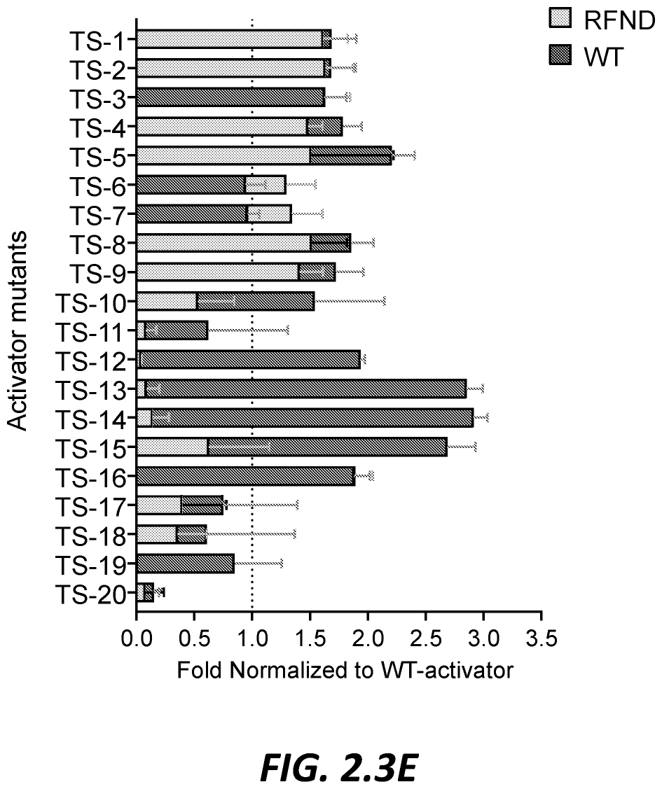

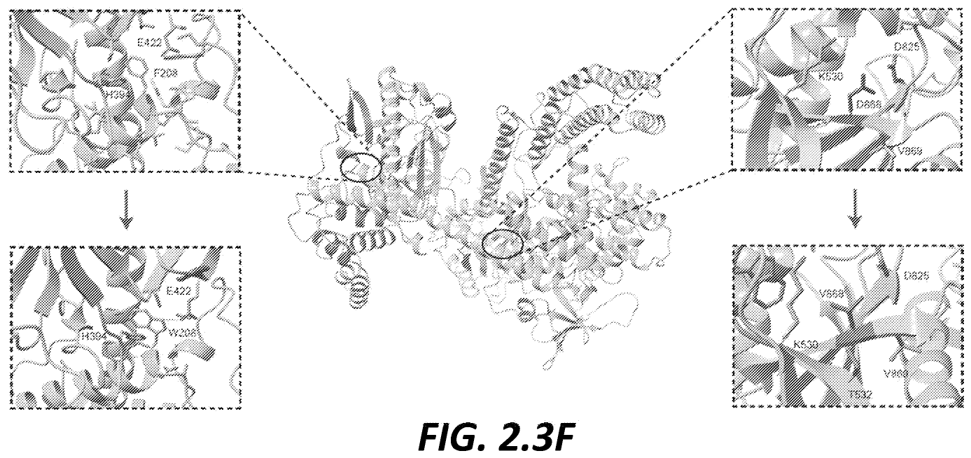

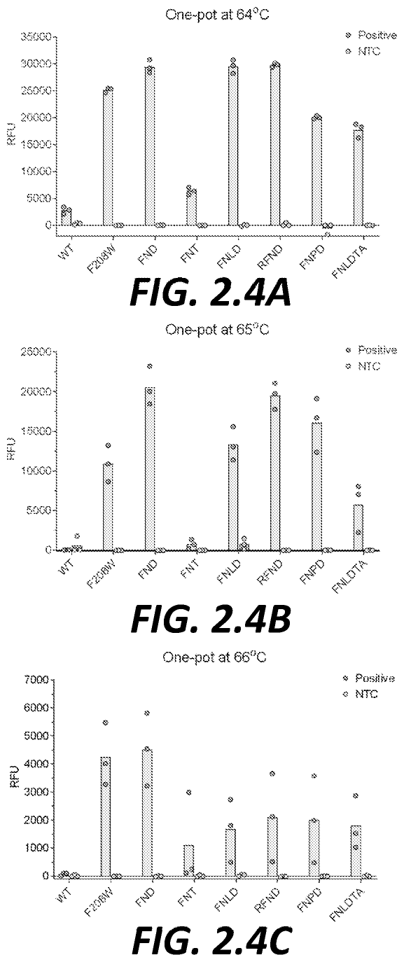

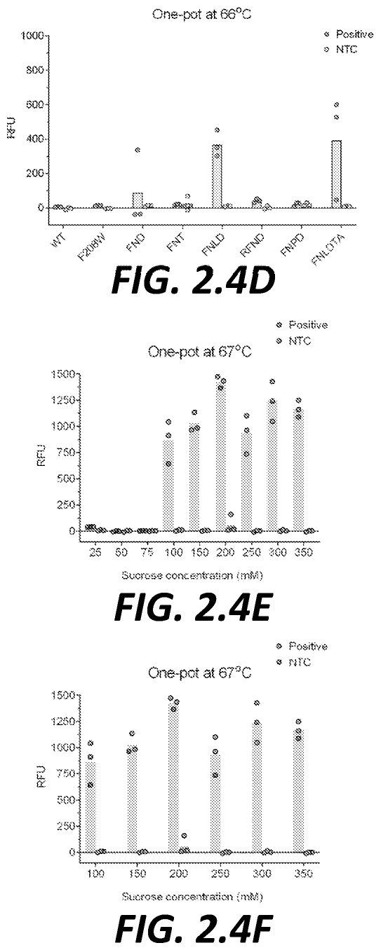

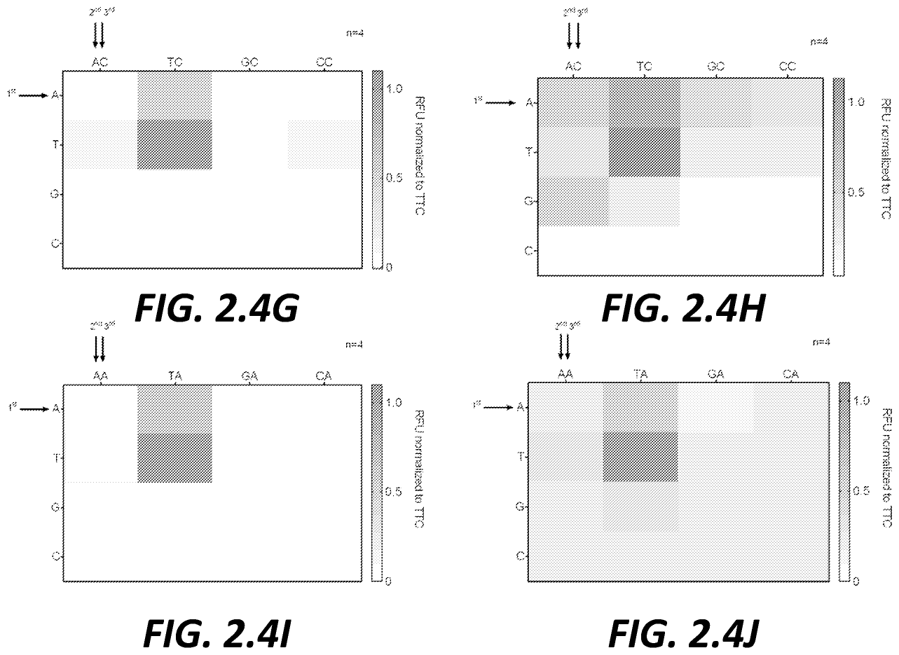

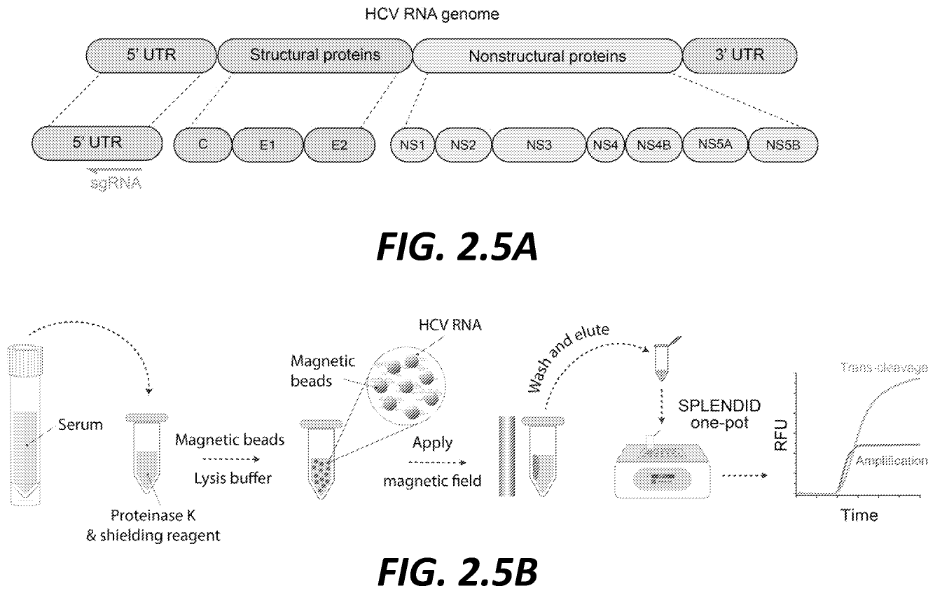

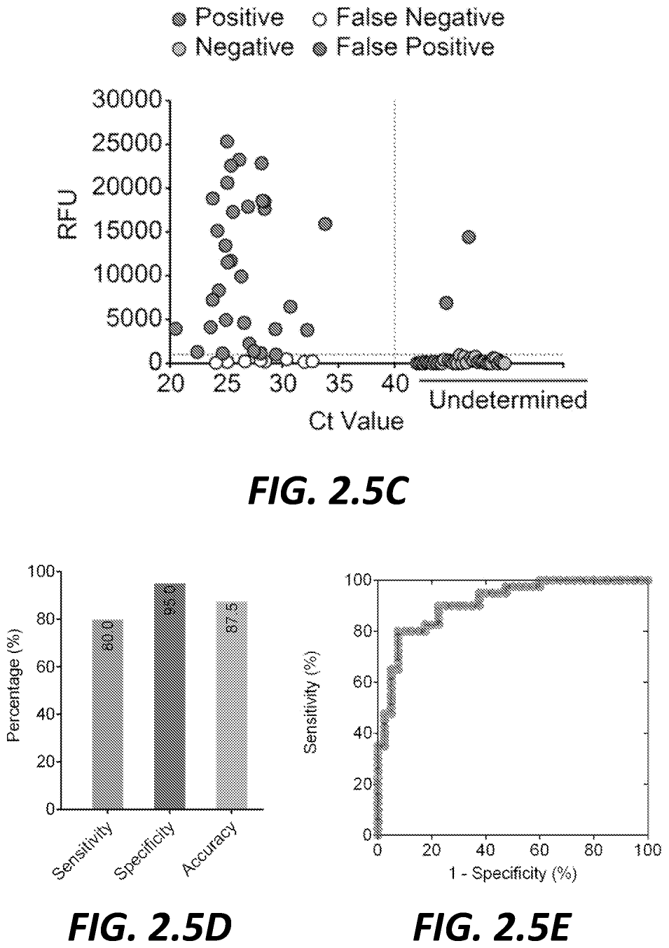

To build a portable detection device that is capable of multiplexing (in this study the device enables monitoring both LAMP amplification and BrCas12b collateral cleavage), the optical system was assembled with two wavelengths. The first one was a MCPCB-mounted LED on 490 nm with a bandwidth of 26 nm, 205 mW driven by 199 mA with impulse length 150 ms for exposure of FITC (Thorlabs). The second LED worked on 625 nm with a bandwidth of 17 nm, 700 mW driven by 215 mA with the same length of impulse for exposure of Cy5 (Thorlabs). The beam of light from the LED was collimated (straightened) by the lens before it travelled to the dichroic mirror and reflected the light into an excitation filter, which cut off the parasitic light. The light beam went to the second dichroic mirror which reflected the beam of light to the collimating lens, concentrating light into the vial with the sample. When the sample started emitting the light from the fluorophore, it then travelled through the collimating lens into the second dichroic mirror, allowing the beam light to go through directly into the emission filter, cutting off the parasitic light. Next, the beam went through another collimating lens prior to being focused and detected by a photodiode sensor/detector (Thorlabs). A schematic diagram of the device is presented in FIG. 2e, and the specifications of device parts are indicated in supplementary table S2.

### Ethics

This study was conducted by strictly following the ethical guidelines approved by the University of Florida Institutional Review Board under the IRB202000633: “Evaluating the Molecular Epidemiology of Coronavirus (COVID-19) in Florida”, IRB202002224: “Saliva Collection to Support Disease Diagnostics”, and IRB202000781: “Detecting SARS-CoV-2 RNA in human samples using an engineered CRISPR-based paper-based test”. IRB202000633 and IRB202000781 protocols were approved as an exempt study with de-identified/coded samples under confidentiality agreements signed by investigator (recipient investigator) and the code owner (collecting investigator). The IRB202002224 was approved as a banking study with the informed consent form by following the Institutional Review Board guidelines.

### Statistics

Patient samples containing different VOCs and wild-type strains were randomised and blinded. Data were visualised using GraphPad Prism 8 (GraphPad Software, San Diego, CA) and results were expressed as means±SD.

### Results

Here, we report the development of a complete one-pot RT-LAMP-coupled CRISPR detection reaction using a novel Cas12b derived from unclassified Brevibacillus sp. SYP-B805 (GenBank ID: WP_165214399.1) named BrCas12b. BrCas12b exhibits phenomenal stability at high temperatures (up to 70° C. in optimal buffers) which is suitable for coupling with an RT-LAMP reaction. Notably, BrCas12b is shown to have high collateral cleavage (referred to as trans-cleavage) up to the temperature of 64° C. without the need for supplemental additives. In addition, BrCas12b works robustly in isothermal amplification buffer, which is an ideal scenario for its incorporation into a complete one-pot reaction. The one-pot RT-LAMP-coupled BrCas12b reaction provides two detection checkpoints: 1) amplification by RT-LAMP that can be tracked by SYTO™ dye and 2) BrCas12b: sgRNA complex detecting amplified targets that can emit a different signal by a Fluorescence Resonance Energy Transfer (FRET)-based reporter. This dual-checkpoint one-pot assay provides a highly accurate level of nucleic acid detection. The broad versatility along with the high specificity of BrCas12b enables us to detect SARS-CoV-2 and distinguish its variants of concerns Alpha (B.1.1.7), Beta (B.1.352), Gamma (P1), Delta (B.1.617.2), and Omicron (B.1.1.529). This example also provides two new low-cost detection methods for fluorescence visualisation. The first one requires a colour-filtered lens attached to a mobile phone camera and a handheld flashlight. By engaging the flashlight, samples can be detected in the dark by the naked eye or enhanced and magnified through the use of filters and the camera. The second method utilises an in-house, portable and inexpensive prototype that allows for quantitative dual checkpoints of amplification and BrCas12b trans-cleavage activity using FITC and Cy5 channels.

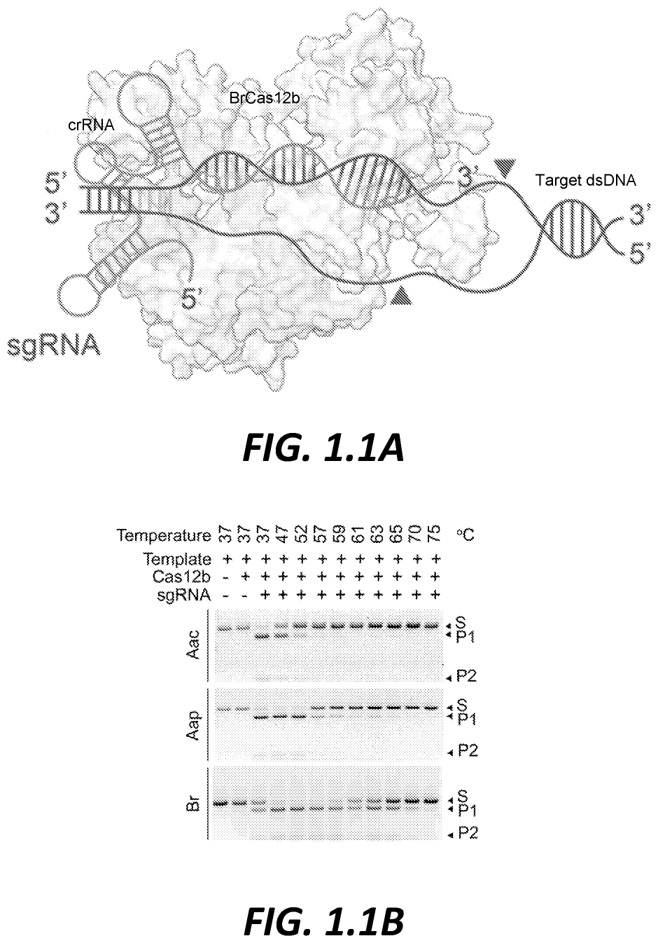

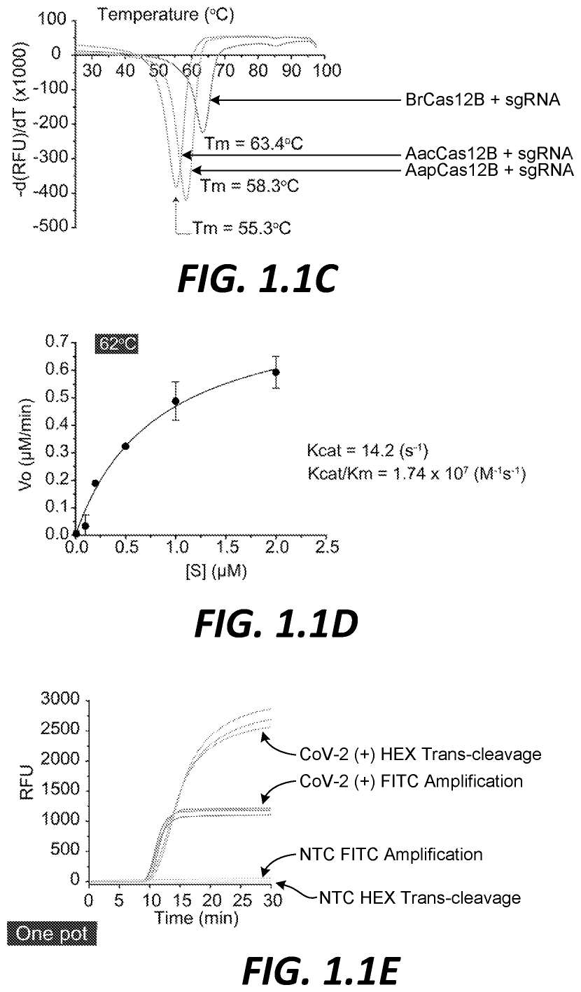

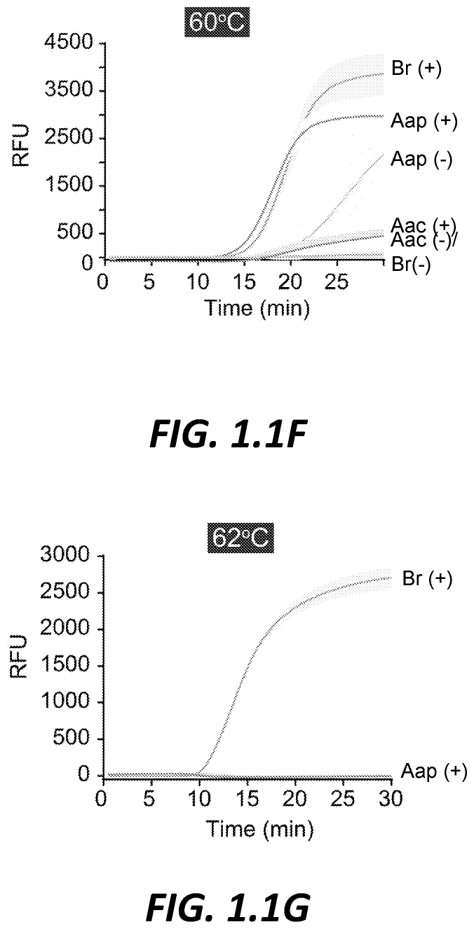

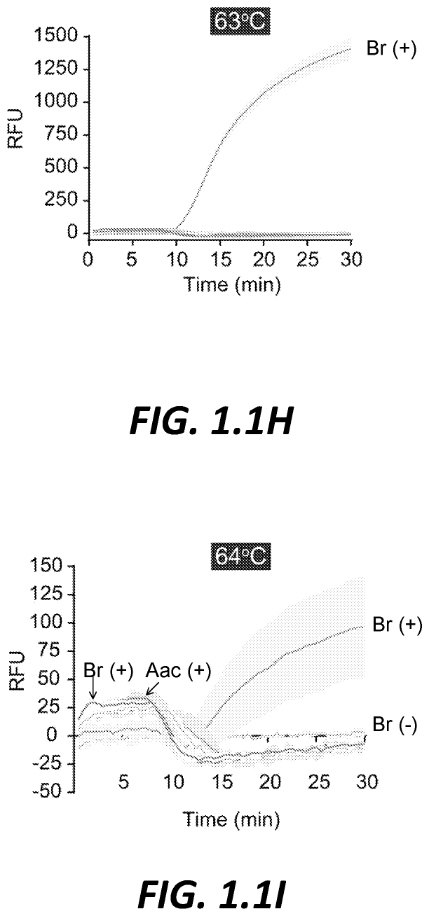

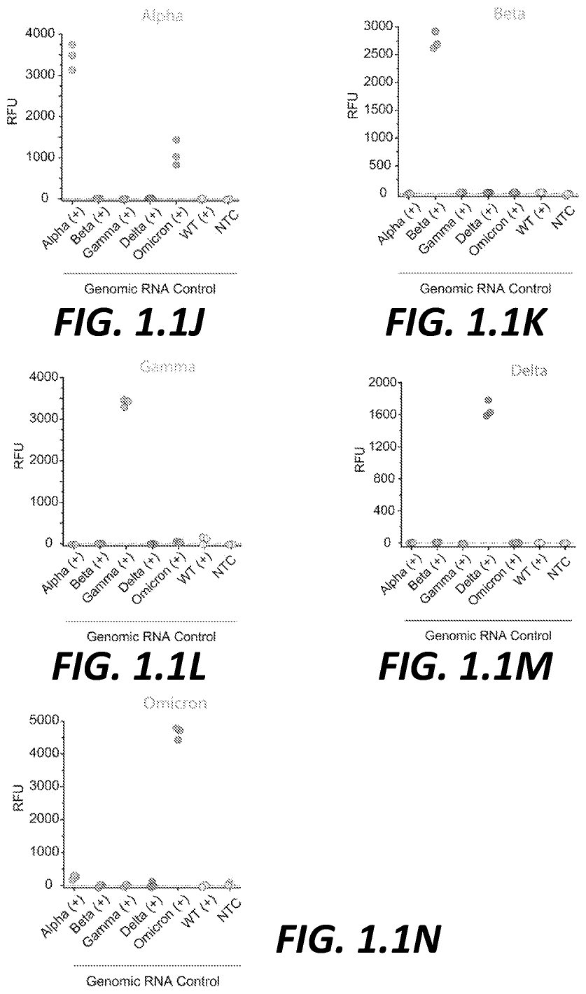

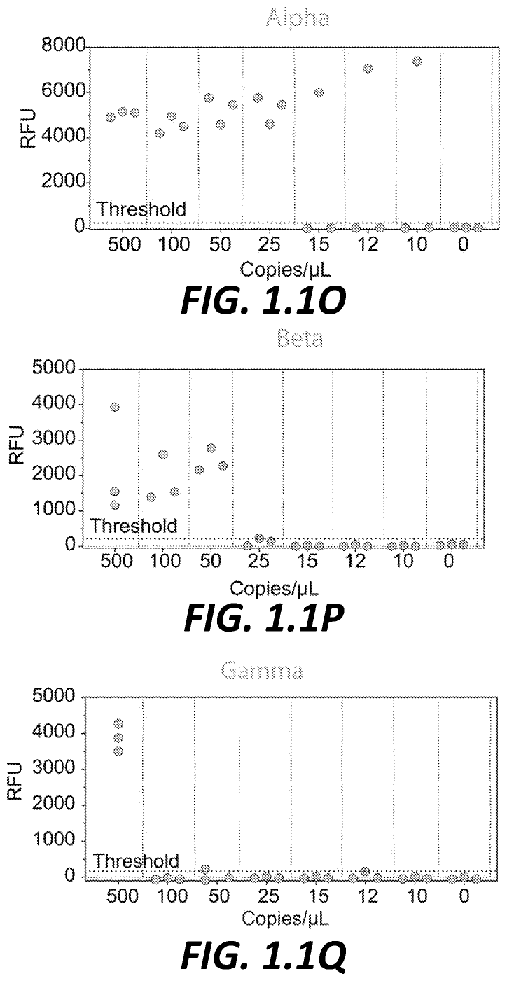

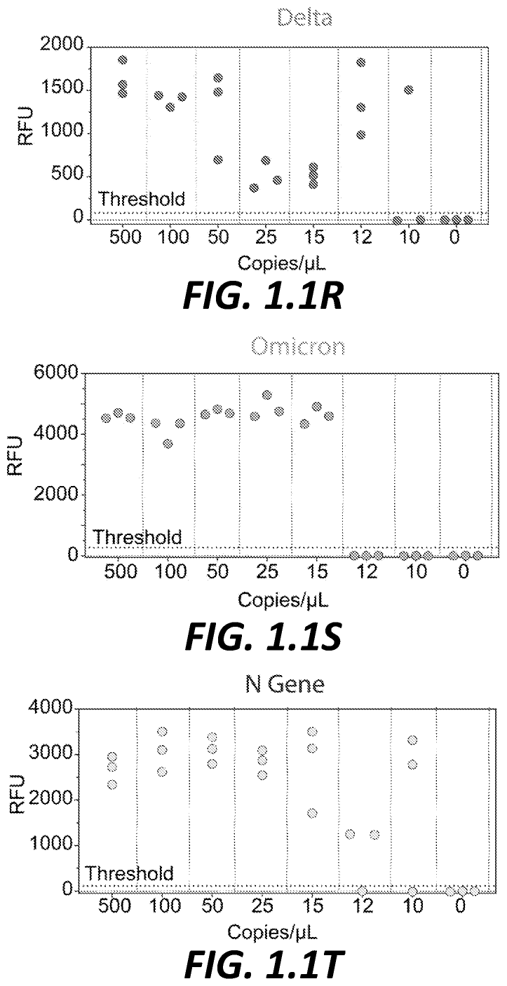

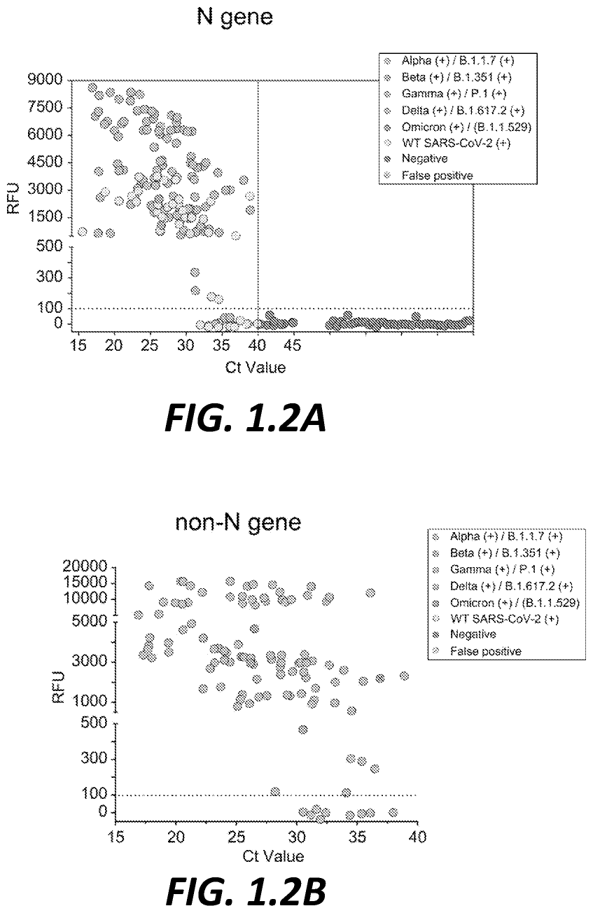

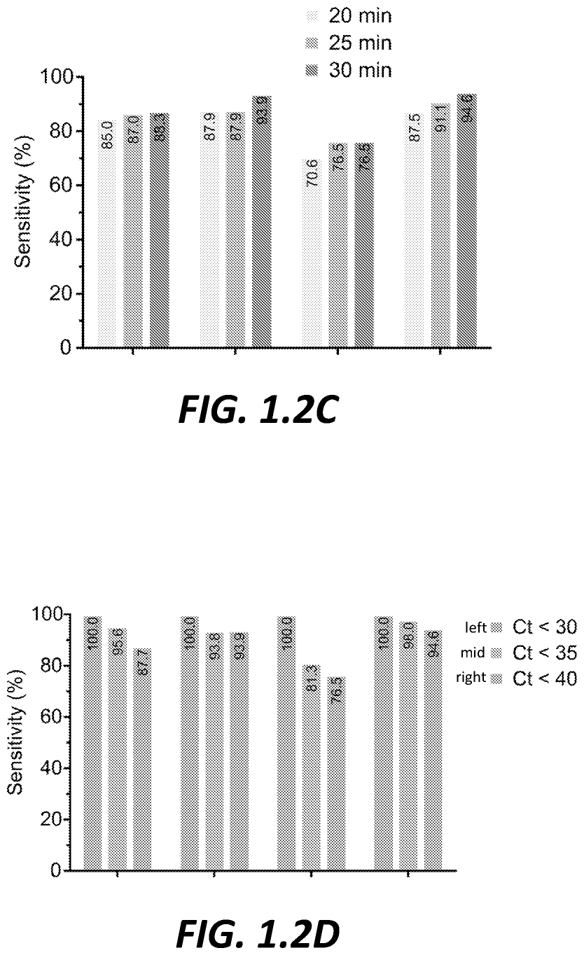

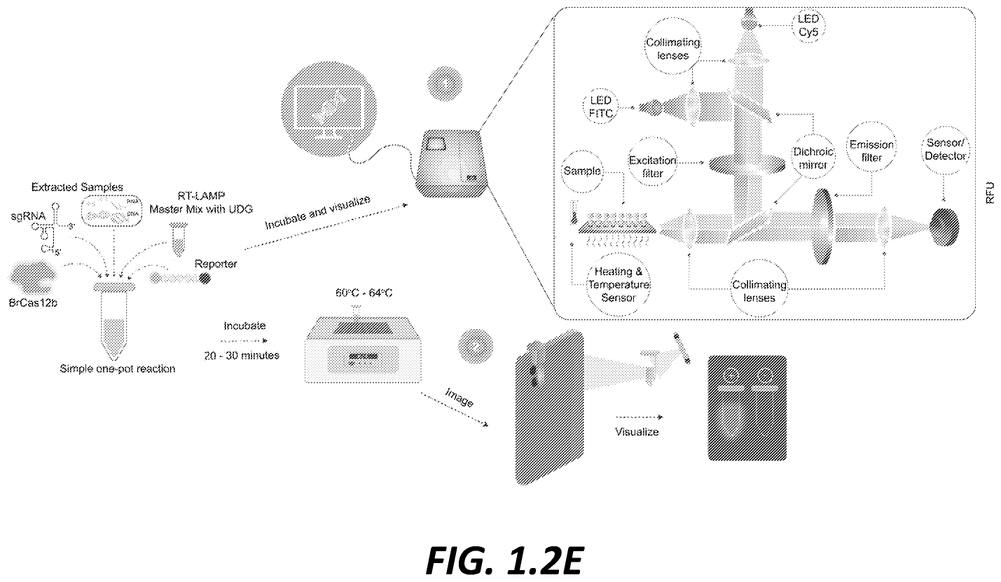

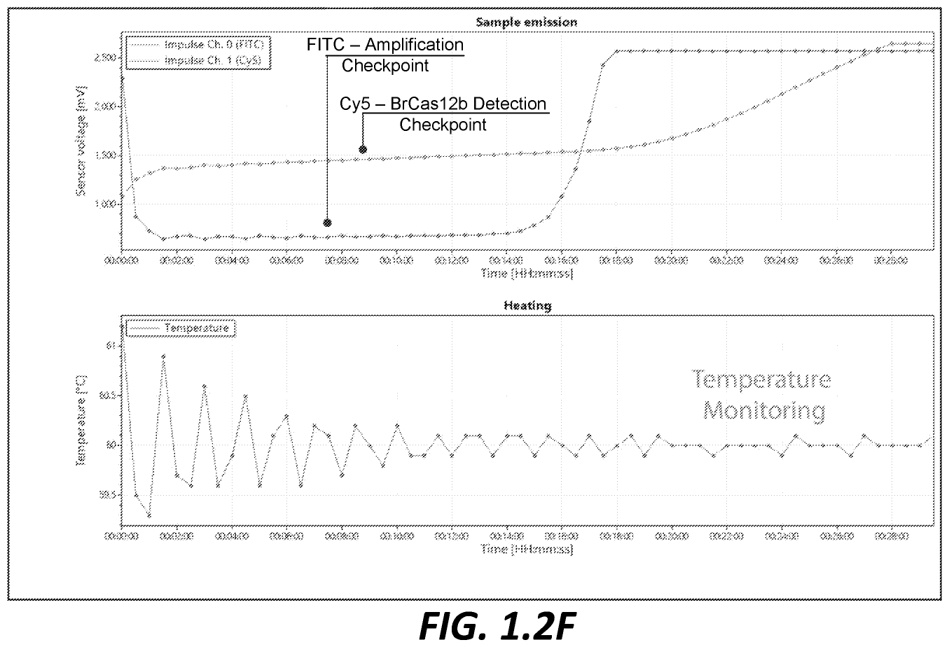

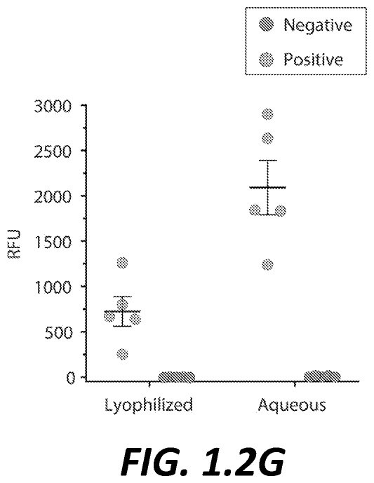

Tian et al. first reported and characterised the BrCas12b from a hot spring bacterium Brevibacillus sp. SYP-B805 that exerts high enzymatic target cleavage (cis-cleavage) activity at elevated temperature (up to 65.5° C.); however, the trans-cleavage activity remained to be investigated (16). BrCas12b forms a complex with crRNA and tracrRNA (˜130-nt) to recognise and cleave dsDNA target containing a TTN PAM sequence upstream of the complementary binding site (FIG. 1.1A). We sought to verify the reported BrCas12b cis-cleavage activity by carrying out a temperature-dependent in vitro cleavage assay compared to Cas12b from Alicyclobacillus acidoterrestris (AacCas12b) and Alicyclobacillus acidiphilus (AapCas12b), two thermostable effector endonucleases with considerable cleavage activity as reported in STOPCovid. A more restrictive PAM sequence TTTN was selected to investigate these three effectors' performance. BrCas12b showed cleavage up to 70° C. in Bovine Serum Albumin-containing buffer compared to 52° C. and 59° C. for AacCas12b and AapCas12b, respectively (FIG. 1.1B). Thermal stability for AacCas12b, AapCas12b, and BrCas12b were confirmed by differential scanning fluorimetry where the melting temperatures were found to be 55.3° C., 58.3° C., and 63.4° C., respectively (FIG. 1.1C). Fascinated by the robust performance of BrCas12b, we proceeded to test the effector's trans-cleavage activity to determine if it is suitable for a one-pot RT-LAMP-coupled detection reaction. As shown by trans-cleavage kinetic analysis, we observed a turnover number of 14.2 s⁻¹ and a catalytic efficiency of 1.74×10⁷ (M⁻¹s⁻¹) at 62° C., indicating fast enzymatic activity (FIG. 1.1D).

The above observations provide advantages when coupling with an RT-LAMP reaction for detection purposes. First, the high optimum temperature of BrCas12b lies within the range of ideal RT-LAMP operating conditions. Additionally, we noticed that BrCas12b performed well in isothermal amplification buffer used in the RT-LAMP reaction. After iterative optimization using many common additives such as proline, taurine, BSA, and betaine, we did not observe further enhancement in the trans-cleavage activity (data not shown). Moreover, FAM- and HEX-based reporters exhibited significantly higher fluorescence signal compared to Cy5-based reporter (data not shown). Therefore, we formulated an RT-LAMP-coupled BrCas12b reaction in the absence of additives and used HEX-based reporter for our assay. By multiplexing the assay with two different reporters, we were able to track both target amplification and BrCas12b trans-cleavage activity in a one-pot reaction. This allowed us to confirm the presence of nucleic acid targets via the two checkpoints. Since the LAMP reaction is prone to non-specific primer-dimer amplification, the BrCas12b trans-cleavage activity serves as a final verdict for detection (FIG. 1.1E). We observed that the trans-cleavage activity of BrCas12b has a 1-3-minute delay after amplification, indicating minimal inhibition by the LAMP reaction on activity. Multiple studies have shown that when combining RT-LAMP/RT-RPA with the CRISPR reaction, the sensitivity of detection is reduced significantly, possibly due to operating temperature differences, an inhibitory effect of excessive amount of amplified product, differences in optimal buffer and salt conditions, and unwanted nonspecific trans-cleavage of Cas effector on primers (1, 17-20). However, having observed such a robust trans-cleavage with a small delay after amplification, we consider that BrCas12b has circumvented many of these issues.

We then systematically tested the one-pot reaction at various temperatures ranging from 60° C. to 64° C. to evaluate detection performance among Cas12b. To compare with STOPCovid, we tested its LAMP primers at 60° C. but with higher concentration of Cas protein and sgRNA. We selected the LAMP primers used in DETECTR for the remaining temperatures because STOPCovid primers are not optimal above 60° C. (FIGS. 1.1F-I) (5). BrCas12b was observed to work robustly up to 63° C. and with reduced activity at 64° C., whereas, as expected, AacCas12b and AapCas12b failed at temperatures above 60° C. (FIGS. 1F-I). Additionally, false positives were occasionally observed when STOPCovid LAMP primers and high concentrations of AapCas12b and sgRNA were used (FIGS. 1.1F).

Thermal stability of BrCas12b enabled us to design LAMP primers with less restrictive parameters compared to STOPCovid to comprehensively distinguish SARS-CoV-2 VOCs from its original strain. We systematically tested each variant including Alpha (B.1.1.7), Beta (B.1.351), Gamma (P1), Delta (B.1.6127.2), and Omicron (B.1.1.529) against one another and the original strain (data not shown). The pair of LAMP primers and sgRNA targeting each variant exhibited high specificity with no relative cross-target detection (FIGS. 1.1J-N) apart from the Alpha variant showing fluorescence signal for the Omicron variant. Prior to the emergence of the Omicron variant in late 2021, we had designed the sgRNA and LAMP primers targeting the Alpha variant based on a 6-base deletion associated with H69del of the spike protein. Coincidentally, multiple sequence alignment revealed that the Omicron variant possesses the same deleted region in its spike protein, resulting in the positive signal when detected with Alpha-targeted LAMP primers and sgRNA. To overcome this nonspecificity, we designed a new pair of LAMP primers and sgRNA targeting the Omicron variant at another unique base-deleted region in the spike protein with high specificity, allowing for the discrimination between the Alpha-detected samples and Omicron-detected samples. For sensitivity testing, we sought to determine the limit of detection (LoD) of nucleocapsid gene (N) for the presence of SARS-COV-2 virions and non-N mutated genes for the prevalent VOCs. The assay confirmed the estimated LoD of 25 copies/μL, 50 copies/μL, 500 copies/μL, 12 copies/μL, 15 copies/μL, and 15 copies/μL for Alpha, Beta, Gamma, Delta, Omicron, and the universal N gene, respectively (FIGS. 1.1 O-T).

We hereafter refer the RT-LAMP-coupled BrCas12b test as CRISPR-SPADE (Single Pot Assay for Detecting Emerging VOCs). We proceeded to validate CRISPR-SPADE in clinical samples including 57 Delta positive (B.1.617.2), 33 Alpha positive (B.1.1.7), 17 Gamma positive (P.1), 1 Beta positive (B.1.351), 45 wild-type SARS-CoV-2, and 53 negative samples. At the time of the study (October 2021), the Omicron variant was not clinically detected. N gene and non-N genes were tested in parallel. The detection of N gene served as a basis for the presence of SARS-CoV-2, whereas the non-N genes were used to differentiate the variants. An 88.3% sensitivity in N gene for all samples was reached (FIGS. 1.2A-C); likewise, a 93.9%, 100%, 76.5%, 94.6%, and 100% sensitivity was achieved after 30 minutes in non-N gene for Alpha, Beta, Gamma, Delta, and Omicron samples, respectively (FIGS. 1.2A-C, Table 1.1). LAMP primers play a crucial role in the sensitivity and specificity of the BrCas12b one-pot detection. Quality in LAMP primer design can aid in the reduction of primer-dimer amplification (15). During the development of the one-pot detection reaction, we occasionally observed non-specific amplification, which was the main cause for false negatives in low copy samples due to the consumption of resources. The high optimum temperature of BrCas12b alleviates some of the LAMP primer design restrictions and thus allows for more flexibility in primer selection. Specifically, in samples with a Cₜ value less than 30, 100% sensitivity was accomplished, and in samples above a Cₜ value of 30, we observed a reduced performance in sensitivity (FIG. 1.2D). With negative samples considered, we achieved an overall 96.7% accuracy and 99.4% specificity with one false positive detected out of a total of 208 samples (Tables 1.1-1.3). Patient samples detected with variants of concern were confirmed through next-generation sequencing (data not shown).

We further developed two methods of fluorescence-based detection: an inexpensive lens attached to a mobile phone camera, and a multiplexing detection prototype engineered to track RT-LAMP amplification and BrCas12b trans-cleavage simultaneously (FIGS. 1.2E-F). The phone-based detection activates via UVA-blue flashlight (410 nm-415 nm) and a combination of low-cost yellow and orange filtered lenses with a clip (cost <$5). This combined lens system allowed for the detection of FRET-based HEX reporters in a dark setting (FIG. 1.2E). We envision that this approach, with proper safety precautions, could be used for home-based testing, as demonstrated by others (3, 4, 19, 21). The portable multiplexing detection prototype was built with two wavelengths in the optical system: a LED working at 490/26 nm excitation for FITC, and a second LED working at 625/17 nm with the same length of impulse for Cy5 excitation. A combination of reporter pairs could be used for this portable device such as SYTO™ 62 using Cy5 channel for tracking amplification and FRET-based FAM reporter using the FITC channel for detection of BrCas12b trans-cleavage activity, or the SYTO™ 9 and Cy5 reporter pair for the opposite channels. This prototype came with a built-in touch screen for ease of use and could potentially be programmed to connect to a mobile phone or a tablet via an app. The device was built with a significant reduction in cost compared to qPCR systems. We anticipate that it will be most suitable in a professional setting such as a clinic.

Additionally, we took one step further by lyophilizing the one-pot reagent to facilitate in transportation, distribution, and deployment without the need for cold-chain requirements in remote or austere settings. We obtained the lyo-ready Warmstart® reagent currently in development from New England Biolabs that is comprised of glycerol-free LAMP reaction mixture. Prior to lyophilization, BrCas12b, sgRNA, and cryoprotectant were added to the lyo-ready mixture. Under our testing conditions, we observed a slightly reduced detection signal from BrCas12b to that of the glycerol version, and the LAMP exhibited a five-minute delay in reaction time (FIG. 1.2K), possibly due to incorporation of several enzymes in the one-pot master mix. Additionally, the freeze-dried reagents were shown to maintain their activity at 4° C. for up to two months (data not shown).

### Discussion

As the COVID-19 pandemic progresses with a growing number of VOCs circulating, the need for a rapid, one-pot detection system to differentiate among the strains is paramount (22-24). Currently, the primary method to reliably detect a VOCs is via next-generation sequencing (NGS) (25-27). Although NGS-based methods are vital in identification and confirmation of new variants, they are labor-intensive and require several hours of processing time, which limit their cost-effectiveness for real time molecular epidemiology surveillance. Due to several advantages of BrCas12b such as high specificity, and robust trans-cleavage at RT-LAMP reaction temperatures, we applied this effector towards a one-pot detection reaction. BrCas12b was observed to have minimal inhibitory effects on RT-LAMP reaction and perform well in isothermal amplification buffer. In ≤30 minutes, samples with high viral load (Cₜ value ≤30) exhibited 100% accuracy, and we achieved >95% sensitivity detecting VOCs for samples with Cₜ value ≤32. When combined with Uracil-DNA Glycosylase (UDG), the one-pot detection reaction minimizes carryover contamination as one false positive were observed in our clinical validation (Table 1.3). Additionally, the freeze-drying process reduces the need for low-temperature logistics. Although lyophilized reactions offer convenience in transport and distribution, there is a slight reduction in fluorescent signal after extended periods of storage. Further research is needed to optimize the lyophilization conditions. We envision that CRISPR-SPADE with the portable detection instrument will move us closer to providing a cost-effective, rapid point-of-care test.

### Tables:

TABLE 1.1
Clinical characteristics of SPADE compared to genomic
sequencing for discriminating SARS-CoV-2 VOC with non-N gene
Clinical characteristics of SARS-CoV-2 VOC with S gene

| VOC | Sensitivity (%) | | Specificity (%) | | Accuracy (%) | | PPV (%) | | NPV (%) | |
|---|---|---|---|---|---|---|---|---|---|---|
| a | 31/33 | (93.9%) | 29/29 | (100%) | 60/62 | (96.8%) | 31/31 | (100%) | 29/31 | (93.5%) |
| b | 1/1 | (100%) | 44/44 | (100%) | 45/45 | (100%) | 1/1 | (100%) | 44/44 | (100%) |
| g | 13/17 | (76.5%) | 42/42 | (100%) | 55/59 | (93.2%) | 13/13 | (100%) | 42/46 | (91.3%) |
| d | 53/56 | (94.6%) | 33/34 | (97.1%) | 86/90 | (95.6%) | 53/54 | (98.1%) | 33/36 | (91.7%) |
| o | 2/2 | (100%) | . . . | | 2/2 | (100%) | 2/2 | (100%) | . . . | |
| Other | 16/16 | (100%) | 28/28 | (100%) | 44/44 | (100%) | 16/16 | (100%) | 28/28 | (100%) |
| All combined | 116/125 | (92.8%) | 176/177 | (99.4%) | 292/302 | (96.7%) | 116/117 | (99.1%) | 176/185 | (95.1%) |

TP = true positive;
TN = true negative;
FP = false positive;
FN = false negative;
PPV = positive predictive value;
NPV = negative predictive value;
Sensitivity = TP/(TP + FN);
Specificity = TN/(TN + FP);
Accuracy = (TP + TN)/(TP + TN + FP + FN);
PPV = TP/(TP + FP);
NPV = TN/(TN + FN)

TABLE 1.2
Clinical validation of CRISPR-SPADE with N gene for detecting SARS-CoV-2
N gene for detecting SARS-CoV-2

| | | Cₜ Value (RT-qPCR) | | | | | | | | |
|---|---|---|---|---|---|---|---|---|---|---|
| | | Positive | | | | | | Negative | | |
| SAR-CoV-2 | | <30 | | <35 | | <40 | | >40 or ND | | Total |
| SPADE | Positive | 96 | (TP) | 130 | (TP) | 136 | (TP) | 0 | (FP) | 136 |
| | Negative | 0 | (FN) | 6 | (FN) | 19 | (FN) | 53 | (TN) | 72 |

| | | | | | |
|---|---|---|---|---|---|
| Total | 96 | 136 | 155 | 53 | 208 |

TP = true positive;
TN = true negative;
FP = false positive;
FN = false negative

TABLE 1.3
Clinical validation of CRISPR-SPADE with non-N gene for detecting SARS-CoV-2

| | S gene for detecting SARS-CoV-2 VOC | | | | | | | | | | | | | |
|---|---|---|---|---|---|---|---|---|---|---|---|---|---|---|
| | | | Genomic sequencing results | | | | | | | | | | | |
| | | | a | | b | | g | | d | | o | Other | | Total |
| SPADE | a | Positive | 31 | (TP) | 0 | (FP) | 0 | (FP) | 0 | (FP) | . . . | 0 | (FP) | 31 |
| | | Negative | 2 | (FN) | 0 | (TN) | 2 | (TN) | 11 | (TN) | . . . | 16 | (TN) | 31 |
| | b | Positive | 0 | (FP) | 1 | (TP) | 0 | (FP) | 0 | (FP) | . . . | 0 | (FP) | 1 |
| | | Negative | 15 | (TN) | 0 | (FN) | 2 | (TN) | 11 | (TN) | . . . | 16 | (TN) | 44 |
| | g | Positive | 0 | (FP) | 0 | (FP) | 13 | (TP) | 0 | (FP) | . . . | 0 | (FP) | 13 |
| | | Negative | 15 | (TN) | 0 | (TN) | 4 | (FN) | 11 | (TN) | . . . | 16 | (TN) | 46 |
| | d | Positive | 1 | (FP) | 0 | (FP) | 0 | (FP) | 53 | (TP) | . . . | 0 | (FP) | 54 |
| | | Negative | 15 | (TN) | 0 | (TN) | 2 | (TN) | 3 | (FN) | . . . | 16 | (TN) | 36 |
| | o | Positive | . . . | | . . . | | . . . | | . . . | | | . . . | | 2 |
| | | Negative | . . . | | . . . | | . . . | | . . . | | . . . | . . . | | . . . |
| | Other | Positive | 0 | (FP) | 0 | (FP) | 0 | (FP) | 0 | (FP) | . . . | 16 | (TP) | 16 |
| | | Negative | 15 | (TN) | 0 | (TN) | 2 | (TN) | 11 | (TN) | . . . | 0 | (FN) | 28 |

| | | | | | | | |
|---|---|---|---|---|---|---|---|
| Total | 94 | 1 | 25 | 100 | 2 | 80 | 302 |

TP = true positive;
TN = true negative;
FP = false positive;
FN = false negative

### REFERENCES FOR EXAMPLE 1

1. Joung J, Ladha A, Saito M, Kim N G, Woolley A E, Segel M, et al. Detection of SARS-CoV-2 with SHERLOCK One-Pot Testing. N Engl J Med. 2020; 383(15):1492-4.
2. Nguyen P Q, Soenksen L R, Donghia N M, Angenent-Mari N M, de Puig H, Huang A, et al. Wearable materials with embedded synthetic biology sensors for biomolecule detection. Nat Biotechnol. 2021.
3. de Puig H, Lee R A, Najjar D, Tan X, Soeknsen L R, Angenent-Mari N M, et al. Minimally instrumented SHERLOCK (miSHERLOCK) for CRISPR-based point-of-care diagnosis of SARS-CoV-2 and emerging variants. Sci Adv. 2021; 7(32).
4. Fozouni P, Son S, Diaz de Leon Derby M, Knott G J, Gray C N, D'Ambrosio M V, et al. Amplification-free detection of SARS-CoV-2 with CRISPR-Cas13a and mobile phone microscopy. Cell. 2021; 184(2):323-33 e9.
5. Broughton J P, Deng X, Yu G, Fasching C L, Servellita V, Singh J, et al. CRISPR-Cas12-based detection of SARS-CoV-2. Nat Biotechnol. 2020; 38(7):870-4.
6. Kaminski M M, Abudayyeh O O, Gootenberg J S, Zhang F, Collins J J. CRISPR-based diagnostics. Nat Biomed Eng. 2021; 5(7):643-56.
7. Abudayyeh O O, Gootenberg J S. CRISPR diagnostics. Science. 2021; 372(6545):914-5.
8. Chen J S, Ma E, Harrington L B, Da Costa M, Tian X, Palefsky J M, et al. CRISPR-Cas12a target binding unleashes indiscriminate single-stranded DNase activity. Science. 2018; 360(6387):436-9.
9. Li S Y, Cheng Q X, Wang J M, Li X Y, Zhang Z L, Gao S, et al. CRISPR-Cas12a-assisted nucleic acid detection. Cell Discov. 2018; 4:20.
10. Li L, Li S, Wu N, Wu J, Wang G, Zhao G, et al. HOLMESv2: A CRISPR-Cas12b-Assisted Platform for Nucleic Acid Detection and DNA Methylation Quantitation. ACS Synth Biol. 2019; 8(10):2228-37.
11. Gootenberg J S, Abudayyeh O O, Lee J W, Essletzbichler P, Dy A J, Joung J, et al. Nucleic acid detection with CRISPR-Cas13a/C2c2. Science. 2017; 356(6336):438-42.
12. Kellner M J, Koob J G, Gootenberg J S, Abudayyeh O O, Zhang F. SHERLOCK: nucleic acid detection with CRISPR nucleases. Nat Protoc. 2019; 14(10):2986-3012.
13. Teng F, Cui T T, Feng G H, Guo L, Xu K, Gao Q Q, et al. Repurposing CRISPR-Cas12b for mammalian genome engineering. Cell Discovery. 2018; 4.
14. Cofsky J C, Karandur D, Huang C J, Witte I P, Kuriyan J, Doudna J A. CRISPR-Cas12a exploits R-loop asymmetry to form double-strand breaks. Elife. 2020; 9.
15. Notomi T, Okayama H, Masubuchi H, Yonekawa T, Watanabe K, Amino N, et al. Loop-mediated isothermal amplification of DNA. Nucleic Acids Res. 2000; 28(12):E63.
16. Tian Y, Liu R R, Xian W D, Xiong M, Xiao M, Li W J. A novel thermal Cas12b from a hot spring bacterium with high target mismatch tolerance and robust DNA cleavage efficiency. Int J Biol Macromol. 2020; 147:376-84.
17. Li Z, Zhao W, Ma S, Li Z, Yao Y, Fei T. A chemical-enhanced system for CRISPR-Based nucleic acid detection. Biosens Bioelectron. 2021; 192:113493.
18. Hardinge P, Murray JAH. Full Dynamic Range Quantification using Loop-mediated Amplification (LAMP) by Combining Analysis of Amplification Timing and Variance between Replicates at Low Copy Number. Sci Rep-Uk. 2020; 10(1).
19. Liu T Y, Knott G J, Smock D C J, Desmarais J J, Son S, Bhuiya A, et al. Accelerated RNA detection using tandem CRISPR nucleases. Nat Chem Biol. 2021; 17(9):982-+.
20. Ali Z, Aman R, Mahas A, Rao G S, Tehseen M, Marsic T, et al. iSCAN: An R T-LAMP-coupled CRISPR-Cas12 module for rapid, sensitive detection of SARS-CoV-2. Virus Res. 2020; 288:198129.
21. Young R M, Solis C J, Barriga-Fehrman A, Abogabir C, Thadani A R, Labarca M, et al. Smartphone screen testing, a novel pre-diagnostic method to identify SARS-CoV-2 infectious individuals. Elife. 2021; 10.
22. Fontanet A, Autran B, Lina B, Kieny M P, Karim S S A, Sridhar D. SARS-CoV-2 variants and ending the COVID-19 pandemic. Lancet. 2021; 397(10278):952-4.
23. Tregoning J S, Flight K E, Higham S L, Wang Z, Pierce B F. Progress of the COVID-19 vaccine effort: viruses, vaccines and variants versus efficacy, effectiveness and escape. Nat Rev Immunol. 2021; 21(10):626-36.
24. Chookajorn T, Kochakarn T, Wilasang C, Kotanan N, Modchang C. Southeast Asia is an emerging hotspot for COVID-19. Nat Med. 2021; 27(9):1495-6.
25. Bhoyar R C, Jain A, Sehgal P, Divakar M K, Sharma D, Imran M, et al. High throughput detection and genetic epidemiology of SARS-CoV-2 using COVIDSeq next-generation sequencing. Plos One. 2021; 16(2).
26. Otu A, Agogo E, Ebenso B. Africa needs more genome sequencing to tackle new variants of SARS-CoV-2. Nat Med. 2021; 27(5):744-5.
27. Chiara M, D'Erchia A M, Gissi C, Manzari C, Parisi A, Resta N, et al. Next generation sequencing of SARS-CoV-2 genomes: challenges, applications and opportunities. Brief Bioinform. 2021; 22(2):616-30.

### Example 2—Engineered Highly Thermostable Cas12b Via De Novo Structural Analyses for One-Pot Detection of Nucleic Acids

The present example provides an engineered Cas12b system from Brevibacillus (eBrCas12b) with improved thermostability that falls within the optimal range of the Reverse Transcription-Loop-Mediated Isothermal Amplification (RT-LAMP) (60° C.-65° C.). Using the de novo structural analyses via DeepDDG and HotSpot Wizard based on Alpha Fold and Swiss Model predicted structures, mutations were introduced into the REC and RuvC domains of wild-type BrCas12b to tighten the hydrophobic core of the protein, thereby enhancing its stability at high temperatures. The assay utilizing eBrCas12b, which was coined SPLENDID (Single-pot LAMP-mediated engineered BrCas12b for nucleic acid detection of infectious diseases), exhibits robust trans-cleavage activity up to 67° C. in a one-pot setting, which significantly, is 4° C. and 7° C. higher than wild-type BrCas12b and AapCas12b, respectively. SPLENDID was further validated clinically in 40 Hepatitis C (HCV) positive and 40 negative serum samples. A sensitivity of 80%, a specificity of 95%, and an accuracy of 87.5% were achieved. Results can be obtained via the one-pot testing in as little as 20 minutes. With the extraction process, the entire assay can be performed in under an hour. Therefore, SPLENDID provides a widely universal planform for detection of infectious diseases.

### Introduction

Clustered Regularly Interspaced Short Palindromic Repeats (CRISPR)-based diagnostics have elevated nucleic acid detection in terms of sensitivity, specificity, and rapidness. CRISPR-Cas systems can be combined with a pre-amplification step in a one-pot reaction to simplify workflow and reduce carryover contamination. Since the beginning of the COVID-19 pandemic, CRISPR-based diagnostic platforms have emerged as a prominent detection technology that could tackle the emergence of future pathogens via massive surveillance testing and potentially replace the traditional time-consuming Reverse Transcription—quantitative Polymerase Chain Reaction (RT-qPCR) method¹⁻⁵. Recent advancements in CRISPR-based tests allows for the combination of a preamplification step, such as reverse transcription loop-mediated isothermal amplification (RT-LAMP), and a CRISPR reaction into a convenient one-pot detection assay⁶. RT-LAMP is a highly sensitive reaction that can result in detectable signals in as fast as 5-10 minutes⁷. However, since the optimal temperature for the RT-LAMP reaction is around 60° C.-65° C.⁷⁻⁹, there has not been a thermostable CRISPR system that encompasses this range. Recently reported STOPCovid technology employed a thermostable Cas12b from Alicyclobacillus acidiphilus (AapCas12b), but one drawback of the system is that the Cas protein ceases to display enzymatic activity at temperatures higher than 60° C.⁶. In Example 1 above, we implemented a thermostable Cas12b, which is derived from an unclassified Brevibacillus (BrCas12b) and exhibited high enzymatic activity up to 63.4° C., within a single-pot CRISPR-diagnostic assay for detection of nucleic acids. Our one-pot assay successfully distinguished among multiple SARS-CoV-2 Variants of Concerns (VOCs) with a high sensitivity and specificity within 20 minutes¹⁰. However, while BrCas12b allowed for detection at a higher temperature than AapCas12b, the range of detection is still narrow. Since RT-LAMP primers are substantially dependent on temperature due to their loop-forming nature, single degree changes often result in significant improvement to target amplification and sensitivity^{7, 11}.

To allow for enzymatic activity at high temperatures such that complete coverage of the optimal range of RT-LAMP could be attained, we employed de novo structural analyses to engineer a more thermostable BrCas12b to allow for better flexibility within the use of the one-pot assay. We employed the capability of AlphaFold and SwissModel to obtain predicted structures of wild-type BrCas12b for the visualization of arrangement and manipulation of amino acid interaction¹²⁻¹⁷. These structures were inputted into stability prediction tools HotSpotWizard and DeepDDG, which could help us gain insight into potential mutations that encourage thermostabilityl^{18, 19}.

The present example provides engineered BrCas12b variants (eBrCas12b) that show up to a 13-fold increase in activity and a melting temperature of 10° C. and 6° C. higher than AapCas12b and wild-type BrCas12b, respectively. When combined in a one-pot reaction with RT-LAMP, the engineered BrCas12b with the highest thermostability was observed to have robust detection capability up to 67° C. Since RT-LAMP is typically performed between 60-65° C., the engineered BrCas12b variant enabled the combination of the RT-LAMP and CRISPR trans-cleavage assays to be carried out at the upper temperature range, allowing for more versatility and ease of primer design. The one-pot assay, which we coined SPLENDID (Single-pot LAMP-mediated engineered BrCas12b for nucleic acid detection of infectious diseases) can be performed in low-resource areas and offers great sensitivity and specificity in as little as 20 minutes.

### Results

### Identification of Stabilizing Mutants of BrCas12b

Type V and type VI CRISPR-Cas systems such as Cas12a, Cas12b, and Cas13a-d have been harnessed for detection of nucleic acids^{1, 20-23}. While Cas12a and Cas13a have been extensively deployed for on-field applications, Cas12b has recently emerged as a distinct class of enzyme, the majority of which have been found to be thermostable²⁴⁻²⁶. Unlike Cas12a, Cas12b requires both a crRNA and tracrRNA to carry out cleavage of double-stranded DNA (dsDNA) and subsequent collateral cleavage of single-stranded DNA (ssDNA) (referred to as trans-cleavage activity) (FIG. 2.1B)^{24, 26}. Although CRISPR-Cas systems can detect nucleic acids, they suffer from low sensitivity that lies within the picomolar range^{21, 27}. Traditionally, CRISPR-based detection platforms are coupled with a pre-amplification step to increase the sensitivity of detection^{1-3, 20}. Amplification-free methods have also been developed and successfully executed^{28, 29}. However, they require multiple guide RNAs to have detectable signal readouts, and as a result, it can be challenging for these assays to distinguish subvariants for genotyping purposes.

Reverse transcription—loop-mediated isothermal amplification (RT-LAMP) has emerged as a powerful tool for molecular diagnosis due to its rapid and isothermal amplification as quick as 5-10 minutes⁷. Since four to six primers are needed to achieve such high sensitivity, false positives that often result from primer-dimer formation are the major limitation of this technology^{30, 31}. Recent efforts have coupled RT-LAMP with a thermostable CRISPR-Cas complex in a one-pot reaction to overcome this drawback⁶. The programmability of the CRISPR-Cas systems improves the specificity of detection. Upon amplification, correct base-pairing between the guide RNA and the amplified target serves as an additional checkpoint to provide accurate signal readouts. The one-pot reaction further eliminates carryover contamination that is frequently observed with two-pot RT-LAMP based platforms¹⁰. Most RT-LAMP reactions work optimally between 60° C.-65° C.^{7, 11}; however, only a handful of Cas enzymes are functional at this temperature range, most of them not very effectively^{6, 32}. The discrepancy in reaction conditions and inhibitory effects between amplification and trans-cleavage has also been a major hurdle in developing such one-pot systems. Recently, we reported the use of a thermostable Cas12b from an unclassified Brevibacillus (BrCas12b) that outperformed previous platforms in a one-pot detection setting. Nevertheless, the thermostable nature of BrCas12b only allowed this system to work robustly up to 63° C.¹⁰.

Previous studies have shown that engineering the crRNA for Cas12a enhances its trans-cleavage activity^{27, 33}. However, the thermostability of Cas enzymes is mainly linked to their secondary and tertiary structures^{34, 35}. Therefore, rather than focusing on modifying sgRNA for BrCas12b, we aimed to engineer the enzyme itself not only to boost its activity but also its thermostability for better synergy with the RT-LAMP reaction. We employed structure-guided rational design to identify amino acid positions whose mutations are likely to be beneficial. To accomplish this, we predicted the structure of BrCas12b utilizing both Alpha Fold and SWISS-MODEL (FIG. 2.1A) followed by sequence alignment of these models with the most homologous solved Cas12b ortholog, BthCas12b (PDBID: 5WTI)^{12-15, 17, 36}, to identify functional domains. Overall, the Alpha Fold and SWISS-MODEL predictions were congruent with one another except for two minor regions close to the RuvC domains (FIG. 2.1B). We next processed these predicted models via DeepDDG, a neural network-based algorithm to generate a fitness landscape of all amino acid positions¹⁹. We observed that the majority of the mutations were destabilizing except for hydrophobic substitutions in the REC1, RuvC, and UK-I regions (FIG. 2.1C). Based on Gibbs free energy changes from the fitness landscape, we analyzed these mutations individually on the AlphaFold structures to determine predictors of thermostability in terms of hydrophobicity, compactness, hydrogen bonding, and salt bridges. In addition to these thermostability factors, we also applied a semi-rational protein design tool called HotSpot Wizard 3.0 to filter down to the best candidates for further validation¹⁸. We pooled in a few single-point mutations that were predicted to be destabilizing according to their ΔΔG values as controls. We identified three prime candidates for point mutations (F208, L795I, T874) from consensus of both algorithms and computed their mutational landscape exhibiting probability of preservation of function (FIGS. 2.1D-2.1F).

### Engineered BrCas12b Showed Robust Activity at High Temperature

34 single-point mutated Br variants were expressed and purified, and we characterized their thermostability and catalytic activity at different temperatures (FIG. 2.2A-2.2C). Of these 34 mutants, we observed that 16 increased the melting temperatures (Tₘ) relative to the wild-type BrCas12b, whose apo form had a Tₘ=61.4° C. Five variants (F208W, D567W, D868I, D868V) displayed an increase of more than 2° C. in their Tₘ with respect to the wild-type. To investigate whether a combination of these stabilizing mutations would display an additive effect on the overall stability of the protein, we stacked beneficial mutations on top of each other to create a library of multiple stacked mutants. We observed that the majority of stacked mutations such as F208W/D868V (FD), F208W/N524V/D868V (FND), F208W/N524V/T874H (FNT (H)), and F208W/N524V/L795l/D868V (FNLD) exhibited an overall additive increase of Tₘ. However, there were a few exceptions. For instance, F208W/N524V/T874S (FNT(S)) and F208W/N524V/L795l/D868V/T874S/A1015E (FNLDTA) decreased thermostability upon stacking.

We performed an in vitro trans-cleavage assay on all variants to check for functional preservation at elevated temperature. While the wild-type BrCas12b variants ceased to work above 65° C. (possibly due to denaturation at high temperature), the engineered BrCas12b variants FN, FND, FNT(S), FNLD, RFND, and FNLDTA showed robust activity up to 68° C. (FIG. 2.2D). We also observed that some stabilizing mutants like D567W, T874H, and D951F were no longer active; by examining the predicted structure, we learned that these mutations were part of the RuvC and UK-I regions and potentially had an active role in Cas12b-mediated cleavage activity. We next sought out to characterize the accuracy of both the AlphaFold and SWISS-MODEL predictions by comparing the computationally predicted single-point mutants with the experimental observations. For the SWISS-MODEL, only 12 out of 22 mutants were experimentally consistent to the software calculations yielding a prediction accuracy of 54%. On the other hand, the AlphaFold model resulted in 14 out of 22 correct mutants yielding a higher prediction accuracy of 64% (FIG. 2.2E,F).

Engineered BrCas12b Exhibited Improved Thermostability while Maintaining Specificity Compared to its Wild Type

We next carried out time-dependent trans-cleavage assays to investigate the stability of BrCas12b variants compared to its wild-type. Ribonucleoprotein complexes (RNP) were incubated between 10 and 60 minutes at different temperature ranging from 60° C. to 75° C. The dsDNA target and fluorescence-based reporter were then added to the RNP complex to initiate the non-specific ssDNA cleavage. Based on kinetic measurements, we observed that the FND and RFND variants showed robust trans-cleavage activity at 68° C. when the RNP complex was incubated for up to 20 minutes and 50 minutes, respectively. On the other hand, the wild-type BrCas12b failed to have detectable trans-cleavage activity in the same conditions (FIG. 2.3A-2.3C). To investigate the specificity of these BrCas12b variants, we designed the dsDNA target with single-point or double-point mutations across its first 10 bases proximal to the PAM site. While the FND variants showed comparable specificity to the wild-type BrCas12b, the RFND displayed a much lower tolerance to double-point mutations near the seed region than wild-type BrCas12b (FIG. 2.3D-E). Overall, the RFND variant exhibited the most thermostability and specificity among all the variants tested.

It was believed that the mutations F208W, N524V, and D868V contributed to the increase in melting temperature while the R160E mutation enhanced the enzyme activity. By looking at the BrCas12b predicted structure closely, we observed that the tryptophan at position 208 further stabilized the interaction with His394 and formed an extra hydrogen bond with Glu422. Through biochemical assays, this mutation was observed to be the most contributing factor to the overall thermostability of the enzyme. The Valine at position 868 allowed for improved compactness in the hydrophobic core of the RuvC region by interacting with more hydrophobic residues around it (FIG. 2.3F). For the mutation R160E, we did not observe an increase in the melting temperature via differential scanning fluorimetry; however, its trans-cleavage activity was higher than the wild-type BrCas12b. We believe conversion of R160E to a negatively charged residue may have subsequently led to the enhancement in dsDNA target binding.

### Engineered BrCas12b Show Robust Activity Up to 67° C. In a RT-LAMP-Mediated One-Pot Reaction

We aimed to investigate the thermostability of these engineered BrCas12b in a one-pot setting. To accomplish this, we employed the LAMP primers described in Broughton et al. that were used to detect SARS-CoV-2 N-gene and tested them at high temperatures ranging from 62° C. to 70° C. with an increment of 1° C. Interestingly, these primers worked robustly up to 68° C. We next coupled some of the most promising BrCas12b variants with the RT-LAMP reaction in a single pot at different temperatures ranging from 64° C. to 68° C. Notably, FND, FNLD, RFND, FNPD, and FNLDTA showed high detection signal at 64° C. with the FND, FNLD, and RFND exhibiting approximately 7-fold in activity compared to the wild-type BrCas12b (FIG. 2.4A). At 65° C., the wild-type BrCas12b completely ceased to work mainly because of its denaturation at high temperature (FIG. 4.4B). The engineered BrCas12b variants worked robustly up to 66° C. but performance declined for all but FNLD and FNLDTA at around 67° C. possibly due to reaching its thermostability limit (FIG. 2.4C,2.4D).

Recent studies have shown that having additives can boost the activity of CRISPR reaction in a one-pot setting^{6, 37-40}. For instance, Joung et al. used taurine in their STOPCovid reaction and observed enhanced detectable signals⁶. Proline was also added to boost Cas12a activity in a two-pot setting³⁸. Therefore, we sought to leverage the one-pot reaction using BrCas12b variants by exploring several additives such as taurine, mannitol, sucrose, trehalose, and betaine. We selected to FNLDTA variant for testing as it showed highest fluorescence signal at 67° C. (FIG. 2.4D). We discovered that sucrose at a final concentration of 200 mM greatly enhanced eBrCas12b enzymatic activity up to approximately 2.5-fold (FIG. 2.4E). We then applied 200 mM sucrose to the rest of the variants and found that sucrose was able to revive their activity at 67° C. (FIG. 2 4F). We therefore coined the assay in which the RFND and optimized sucrose condition were used in one-pot setting SPLENDID (Single-pot LAMP-mediated engineered BrCas12b for nucleic acid detection of infectious diseases).

CRISPR-Cas12b-mediated cleavage and detection of dsDNA are dependent on the presence of a short protospacer adjacent motif (PAM) upstream of the target sequence. It was empirically determined that the canonical PAM for BrCas12b was TTN^{24-26, 41}. However, we speculated that when targeting single-stranded DNA (ssDNA), a PAM sequence may not be required as it shares similar enzymatic activity with Cas12a²¹. Since the RT-LAMP reaction can result in single-stranded DNA byproducts due to its loop-forming nature at elevated temperatures⁷, we hypothesized that PAMless or near-PAMless detection could be achieved by our RT-LAMP-coupled one-pot assay. This can potentially alleviate some challenges in primer and guide RNA designs. To investigate the PAM-dependency of eBrCas12b, we designed a PAM library of dsDNA activators that were comprised of all possible 3-nucleotide PAM sequences (NNN). We first sought to test the trans-cleavage of the RFND variant against these PAM combinations without the RT-LAMP step. We observed that the TTN PAM containing activators displayed the highest activity with engineered Cas12b, consistent with previous studies. We also noticed significant trans-cleavage activity with activators containing the ATA, ATT, and ATC PAM but not with ATG. Similarly, TAA, TAT, and TAC containing activators had low levels of detection, but not TAG. Our results indicate that the presence of a single T at either the first or the second position is sufficient to initiate the trans-cleavage of eBrCas12b provided that the position 3 contains A, T, or C. We next tested the detection of all the PAM-library activators in a one-pot setting (with RT-LAMP amplification). We observed that while the TTN PAM containing activators had the best detection, a large number of non-canonical PAM containing sequences also showed detection, albeit at lower levels—the only exceptions being CTC, CGC, and CCC PAMs, which showed no detection. Our results demonstrate that it is possible to target a variety of non-canonical PAM sequences if amplified by RT-LAMP in a one-pot assay.

### Validation of SPLENDID in Hepatitis C Infected Samples

Screening for Hepatitis C (HCV) is of importance for early treatment since there are no vaccines available⁴²⁻⁴⁴. Individuals infected with HCV can be cured within 8 to 12 weeks when detected early; otherwise, cirrhosis, hepatocellular carcinoma, and potential death can occur if left unnoticed^{44, 45}. Traditional antibody assays offer up to 98% sensitivity and 99% specificity; however, these assays cannot distinguish between past infection and active infection. On the other hand, quantitative HCV RNA tests also exhibit similar sensitivity, specificity, and low limit of detection (10-15 IU/mL)⁴⁶⁻⁴⁸, but these detection platforms are laborious, time-consuming, and uneconomical. Therefore, as a proof of concept, we sought to develop a simple rapid test for Hepatitis C (HCV) using SPLENDID.

Prior to testing, single-guide RNA was designed to target the 5′ untranslated region (5′ UTR) of the HCV genome (FIG. 2.5A). We then proceeded to clinically validate the SPLENDID in 80 human serum samples (40 HCV-infected samples and 40 samples that had tested negative for HCV). Crude samples were processed via a magnetic-based extraction to isolate viral RNA. These samples were randomized and blinded before undergoing the SPLENDID testing (FIG. 2.5B). All fluorescence-based measurements were compared to the RT-qPCR results to determine the sensitivity, specificity, and accuracy. Out of 80 samples, we observed 8 false negatives and 2 false positives, achieving a sensitivity of 80%, a specificity of 95%, and an accuracy of 87.5% (FIGS. 2.5C, D). When analyzing the patient sample fluorescence results via a receiver operating characteristic curve (ROC) for the SPLENDID assay at t=25 minutes, we observed that the area under the curve (AUC) was 91%, indicating a high threshold for differentiation between positive and negative samples (FIG. 2.5E).

### Methods

### Nucleic Acid Preparation

Double-stranded DNA targets, single-guide RNAs, and fluorescence-quencher reporters were synthesized by Integrated DNA Technologies (IDT). PAM Library targeting SARS-CoV-2 N gene was designed and synthesized by Twist Bioscience. For trans-cleavage assay without RT-LAMP, the target-strand (TS) and non-target-strand (NTS) activators were ordered as 60-mer ssDNA oligos and annealed in 5:1 (NTS: TS) ratio in 1× nuclease-free duplex buffer (IDT).

### Site-Directed Mutagenesis

The wild-type BrCas12b gene were codon-optimized for E. coli expression was synthesized by Twist Bioscience and cloned into pET28a⁺ vector. For a mutant with single-point and double-point mutations, mutagenesis was carried out using the Q5® Site-Directed Mutagenesis Kit (New England Biolabs) following the manufacturer's protocols. For simultaneous triple-point mutations, primers with intended mutations were designed and used to amplify each fragment of the BrCas12b using Q5® polymerase and assembled into a 6×His-MBP destination vector (a gift from Scott Gardia, Addgene #29656) using NEBuilder® HiFi DNA Assembly Kit (New England Biolabs).

### Protein Expression and Purification

Expression and purification of BrCas12b variants was performed according to a previous study with minor modifications¹⁰. After transforming BrCas12b variants into Rosetta™ 2(DE3)pLysS Singles Competent Cells (Milipore Sigma), individual colonies were selected and propagated overnight (12-15 hours) within 10 mL of Luria Broth (Fischer Scientific) containing the appropriate antibiotics. The bacterial cultures were then scaled up within a media of 2 L of Terrific Broth containing the corresponding antibiotics and a few drops of antifoam 204 (Sigma Aldrich). Once an OD600 of 0.5-0.8 was attained, the culture was cooled in an ice bath for 30 mins, induced with 0.2 mM IPTG (isopropyl b-d-1-thiogalactopyranoside), and shaken at 18° C. overnight (14-18 hours). The bacterial cells were then pelleted (39,800×g at 4° C.), diluted in lysis buffer (0.5 M NaCl, 50 mM Tris-HCl, PH=7.5, 1 mM PMSF, 0.5 mM TCEP, 0.25 mg/ml lysozyme, and 10 mg/mL deoxyribose nuclease I), and disrupted by sonication. The lysed cells were then spun down (39,800×g at 4° C.), and the supernatant was then subjected to suction filtration through a 0.22-micron filter (Millipore Sigma). The filtered solution was purified using an FPLC Biologic Duoflow system (Bio-rad) through a Histrap 5 mL FF column (Cytiva), which was then washed (wash buffer: 0.5 M NaCl, 50 mM Tris-HCl, PH=7.5, 20 mM Imidazole, 0.5 mM TCEP) before eluting (elution buffer: 0.5 M NaCl, 50 mM Tris-HCl PH=7.5, 300 mM imidazole, 0.5 mM TCEP). For BrCas12b variants that were cloned into the 6×His-MBP destination vector, an extra step of TEV site cleavage was performed to remove the MBP tag, and the protein was processed using Hitrap Heparin HP purification. The elution was evaluated using SDS-PAGE; the fractions containing the protein were consolidated and concentrated (2000×g at 4° C.) in a 30 kDa MWCO Vivaspin® 20 concentrator. The protein was stored in −20° C. for further use, with back-up reserves of each protein kept at −80 after dilution (buffer C: 150 mM NaCl, 50 mM HEPES, pH=7, 0.5 mM TCEP).

### Differential Scanning Fluorimetry

The melting temperature of each protein was mainly carried out in its apo form. BrCas12b was mixed in Protein Thermal Shift™ buffer (Thermofisher) in combination with 1χ reaction buffer (100 nM NaCl, 50 nM Tril-HCl, pH=7.5, 1 mM DTT, and 10 mM MgCl₂) to a final concentration of 500 nM. Protein Thermal Shift dye™ (Thermofisher) was then added to each mixture before being transferred to the qPCR StepOne Plus system (Thermofisher). The temperature profile was observed over a temperature range of 25° C.-80° C. at a ramp rate of 1%/s. Duplicates of each experiment were performed in replicates of two.

### Temperature- and Time-Dependent Trans-Cleavage Assay

The thermostable BrCas12b variants and sgRNA were combined in 1× NEBuffer 2.1 (New England Biolabs) to a final concentration of 50 nM: 100 nM (sgRNA: BrCas12b) and then transferred to a pre-heated CFX96 Real-Time PCR system with C1000 Thermal Cycler module (Bio-rad) using a temperature gradient setting across each row, ranging from 60° C. to 75° C. (60° C., 61° C., 63° C., 65.9° C., 69.5° C., 72.5° C., 74.2° C., and 75° C.). The ribonucleoprotein complexes were incubated from a range 10-60 minutes with an increment of 10 minutes. A fluorescence-based reporter (FQ) and dsDNA activator were then added to each mixture to a final concentration of 250 nM and 1 nM, respectively. The reactions were isothermally incubated at their corresponding complexation temperature for consistency. Fluorescent measurements were taken every 30 seconds for 120 cycles on the HEX channel (λₑₓ: 525/10 nm, λₑₘ: 570/10 nm). This experiment was repeated at incubation temperatures of 67° C. and 68° C. without the gradient setting to cover a better temperature distribution. All experiments were repeated twice.

### One-Pot RT-LAMP-Coupled BrCas12b Detection Assay

LAMP primers were designed using NEB® LAMP Primer Design Tool from New England Biolabs (https://lamp.neb.com/#!/) and PrimerExplorer v5 from Eiken Chemical Co. (https://primerexplorer.jp/e/).

BrCas12b variants, single-guide RNA, fluorescence-quencher reporter, and 10× LAMP primers were combined in 1× Warmstart® Multi-Purpose/RT-LAMP Master Mix with UDG (New England Biolabs) to a final concentration of 200 nM, 400 nM, 2000 nM, and 1× LAMP primers (200 nM F3/B3, 1600 nM FIP/BIP, and 800 nM LF/LB), respectively, yielding a volume of 22 mL. The activator (dsDNA/RNA) was added to the mixture (25 mL final volume), and the reaction was then transferred to CFX96 Real-Time PCR system with C1000 Thermal Cycler module (Bio-rad). Fluorescence measurements were taken every 30 seconds per cycle for 120 cycles.

### PAM Library

For trans-cleavage assay without the RT-LAMP step, BrCas12b and sgRNA activators were combined to a final concentration of 100 nM:50 nM respectively in 1× NEBuffer 2.1 (New England Biolabs) and incubated at 65° C. for 15 min. Fluorescence-based reporter (FQ) and 25 nM activators containing various PAM sequences were added to the reaction mixture containing Cas12b trans-activated complex to a final concentration of 250 nM and 1 nM respectively. The entire reaction was then immediately transferred to a Bio-rad CFX96 Real-Time system with C1000 Thermal Cycler module. The reaction was isothermally incubated at 65° C., and fluorescence measurements were read every 30 seconds per cycle over 120 cycles. In the experiment with RT-LAMP, the one-pot BrCas12b detection assay method monitoring trans-cleavage only was utilized. 3 μL of 10 PM activators containing various PAM sequences were added to the reaction. Both experiments were carried out in duplicates and repeated twice.

### Patient Samples Extraction and Processing

For clinical validation, 80 patient samples consisting of human serum were obtained, forty of which were Hepatitus C positive collected by HCV-TARGET; the other forty deriving from healthy patients were collected by BocaBiolistics. Viral RNA was extracted from serum samples using Quick-DNA/RNA™ Viral MagBead Kit R2140 (Zymo Research) or Dipstick DNA Extraction Kit (Bento). Extracted patient samples were chosen at random and blinded for one-pot testing.

The Quick-DNA/RNA™ Viral MagBead extraction was conducted according to manufacturer protocol. 10 μL of Proteinase K (20 mg/mL) was combined with 200 μL of serum samples in 1.5 mL centrifuge tubes and incubated at room temperature (RT) for 15 minutes. DNA/RNA Shield™ (2× Concentrate) was added to the serum sample containing Protease K in a 1:1 ratio. 800 μL Viral DNA/RNA Buffer was then incorporated into the combined mixture followed by the addition of 20 μL Magbinding Beads™ and vortexing for 10 minutes. The centrifuge tubes were placed in a magnetic stand until the beads pelleted. Supernatant was aspirated and discarded. The beads were washed with 250 μL of MagBead DNA/RNA Wash 1, 250 μL MagBead DNA/RNA Wash 2, and two rounds of 250 μL of 100% ethanol. The tubes containing beads were air-dried for 10 minutes. DNA/RNA was eluted using 30 μL DNase/RNase-Free water and was subjected to BrCas12b detection reaction.

For the dipstick extraction method, the Dipstick DNA Extraction Kit was used according to manufacturer protocol. 100 μL of human serum sample was added to 200 μL of Extraction Buffer in a 1.5 mL microcentrifuge tube. An additional 200 μL of Extraction Buffer was combined to bring the total mixture volume to 500 μL. The uncovered (binding) end of the dipstick was dipped into the mixture three times to ensure that it was thoroughly soaked. The dipstick was then transferred to 1 mL of Wash Buffer in a separate tube and washed 5 times. The dipstick was gently wiped on the tube edge to eliminate the remaining Wash Buffer. The washed dipstick was then dipped up to 15 times in the pre-prepared one-pot RT-LAMP-coupled BrCas12b reaction. The one-pot detection assays were operated at 65° C. in the CFX96 Real-Time PCR system with C1000 Thermal Cycler module (Bio-rad). Fluorescence measurements were taken every 30 seconds per cycle for 120 cycles.

### RT-qPCR of HCV Clinical Samples

HCV specific primers and probe sequences were designed as described in Zauli et. al. The probe was modified by adding an internal ZEN™ quencher from IDT in order to reduce the background signal. Ct values for 40 HCV infected and 40 healthy serum samples were obtained by using Luna® Probe One-Step RT-qPCR 4× Mix with UDG (NEB #M3019S) and following manufacturer's protocol. Briefly, for N samples, the following master mix was prepared:

| | | | |
|---|---|---|---|
| 1 | Luna ® Probe One-Step RT-qPCR 4X Mix with | N × 5 | μL |
| | UDG | | |
| 2 | Fwd primer | N × 0.8 | μL |
| 3 | Rev primer | N × 0.8 | μL |
| 4 | Probe | N × 0.4 | μL |
| 5 | RNase-free water | N × 10 | μL |
| | Total Volume | N × 17 | μL |

Once assembled, the master mix was added to a 96-well plate (Applied Biosystems). 3 μL of extracted patient sample were deposited in each well to initiate the reaction. The 96-well plate was then inserted into the StepOnePlus Real-Time PCR system (Applied Biosystems), which set auto threshold to calculate the Ct values.

### REFERENCES FOR EXAMPLE 2

1. Gootenberg, J. S. et al. Nucleic acid detection with CRISPR-Cas13a/C2c2. Science 356, 438-+ (2017).
2. Broughton, J. P. et al. CRISPR-Cas12-based detection of SARS-CoV-2. Nat Biotechnol 38, 870-874 (2020).
3. Patchsung, M. et al. Clinical validation of a Cas13-based assay for the detection of SARS-CoV-2 RNA. Nat Biomed Eng 4, 1140-1149 (2020).
4. Hampton, T. Virus Surveillance and Diagnosis With a CRISPR-Based Platform. Jama—J Am Med Assoc 324, 430-430 (2020).
5. Welch, N. L. et al. Multiplexed CRISPR-based microfluidic platform for clinical testing of respiratory viruses and identification of SARS-CoV-2 variants. Nat Med 28, 1083-+ (2022).
6. Joung, J. et al. Detection of SARS-CoV-2 with SHERLOCK One-Pot Testing. N Engl J Med 383, 1492-1494 (2020).
7. Notomi, T. et al. Loop-mediated isothermal amplification of DNA. Nucleic Acids Res 28, E63 (2000).
8. Amaral, C. et al. A molecular test based on RT-LAMP for rapid, sensitive and inexpensive colorimetric detection of SARS-CoV-2 in clinical samples. Sci Rep-Uk 11 (2021).
9. Huang, X., Tang, G. Y., Ismail, N. & Wang, X. W. Developing RT-LAMP assays for rapid diagnosis of SARS-CoV-2 in saliva. Ebiomedicine 75 (2022).
10. Nguyen, L. T. et al. A thermostable Cas12b from Brevibacillus leverages one-pot discrimination of SARS-CoV-2 variants of concern. Ebiomedicine 77 (2022).
11. Wong, Y. P., Othman, S., Lau, Y. L., Radu, S. & Chee, H. Y. Loop-mediated isothermal amplification (LAMP): a versatile technique for detection of micro-organisms. J Appl Microbiol 124, 626-643 (2018).
12. Jumper, J. et al. Highly accurate protein structure prediction with AlphaFold. Nature 596, 583-+ (2021).
13. Waterhouse, A. et al. SWISS-MODEL: homology modelling of protein structures and complexes. Nucleic Acids Research 46, W296-W303 (2018).
14. Kopp, J. & Schwede, T. The SWISS-MODEL repository: new features and functionalities. Nucleic Acids Research 34, D315-D318 (2006).
15. Guex, N., Peitsch, M. C. & Schwede, T. Automated comparative protein structure modeling with SWISS-MODEL and Swiss-PdbViewer: A historical perspective. Electrophoresis 30, S162-S173 (2009).
16. Studer, G. et al. QMEANDisCo-distance constraints applied on model quality estimation (vol 36, pg 1765, 2020). Bioinformatics 36, 2647-2647 (2020).
17. Bertoni, M., Kiefer, F., Biasini, M., Bordoli, L. & Schwede, T. Modeling protein quaternary structure of homo- and hetero-oligomers beyond binary interactions by homology. Sci Rep-Uk 7 (2017).
18. Sumbalova, L., Stourac, J., Martinek, T., Bednar, D. & Damborsky, J. HotSpot Wizard 3.0: web server for automated design of mutations and smart libraries based on sequence input information. Nucleic Acids Research 46, W356-W362 (2018).
19. Cao, H. L., Wang, J. X., He, L. P., Qi, Y. F. & Zhang, J. Z. DeepDDG: Predicting the Stability Change of Protein Point Mutations Using Neural Networks. J Chem Inf Model 59, 1508-1514 (2019).
20. Gootenberg, J. S. et al. Multiplexed and portable nucleic acid detection platform with Cas13, Cas12a, and Csm6. Science 360, 439-+ (2018).
21. Chen, J. S. et al. CRISPR-Cas12a target binding unleashes indiscriminate single-stranded DNase activity. Science 360, 436-+ (2018).
22. Li, L. X. et al. HOLMESv2: A CRISPR-Cas12b-Assisted Platform for Nucleic Acid Detection and DNA Methylation Quantitation. Acs Synth Biol 8, 2228-2237 (2019).
23. Qiao, X. et al. Sensitive analysis of single nucleotide variation by Cas13d orthologs, EsCas13d and RspCas13d. Biotechnol Bioeng 118, 3037-3045 (2021).
24. Strecker, J. et al. Engineering of CRISPR-Cas12b for human genome editing. Nat Commun 10 (2019).
25. Tian, Y. et al. A novel thermal Cas12b from a hot spring bacterium with high target mismatch tolerance and robust DNA cleavage efficiency. Int J Biol Macromol 147, 376-384 (2020).
26. Teng, F. et al. Repurposing CRISPR-Cas12b for mammalian genome engineering. Cell Discov 4, 63 (2018).
27. Nguyen, L. T., Smith, B. M. & Jain, P. K. Enhancement of trans-cleavage activity of Cas12a with engineered crRNA enables amplified nucleic acid detection (vol 11, 4906, 2020). Nat Commun 11 (2020).
28. Fozouni, P. et al. Amplification-free detection of SARS-CoV-2 with CRISPR-Cas13a and mobile phone microscopy. Cell 184, 323-+ (2021).
29. Liu, T. Y. et al. Accelerated RNA detection using tandem CRISPR nucleases (vol 17, pg 982, 2021). Nat Chem Biol 17, 1210-1210 (2021).
30. Coelho, B. D. et al. Essential properties and pitfalls of colorimetric Reverse Transcription Loop-mediated Isothermal Amplification as a point-of-care test for SARS-CoV-2 diagnosis. Mol Med 27 (2021).
31. Hardinge, P. & Murray, J. A. H. Reduced False Positives and Improved Reporting of Loop-Mediated Isothermal Amplification using Quenched Fluorescent Primers. Sci Rep-Uk 9 (2019).
32. Ali, Z. et al. iSCAN: An RT-LAMP-coupled CRISPR-Cas12 module for rapid, sensitive detection of SARS-CoV-2. Virus Res 288 (2020).
33. Kim, H. et al. Highly specific chimeric DNA-RNA-guided genome editing with enhanced CRISPR-Cas12a system. Mol Ther-Nucl Acids 28, 353-362 (2022).
34. Hand, T. H., Das, A. & Li, H. Directed evolution studies of a thermophilic Type II-C Cas9. Method Enzymol 616, 265-288 (2019).
35. Kumar, S., Tsai, C. J. & Nussinov, R. Factors enhancing protein thermostability. Protein Eng 13, 179-191 (2000).
36. Wu, D., Guan, X. Y., Zhu, Y. W., Ren, K. & Huang, Z. W. Structural basis of stringent PAM recognition by CRISPR-C2c1 in complex with sgRNA. Cell Res 27, 705-708 (2017).
37. Louwrier, A. & van der Valk, A. Can sucrose affect polymerase chain reaction product formation? Biotechnol Lett 23, 175-178 (2001).
38. Li, Z.H. et al. A chemical-enhanced system for CRISPR-Based nucleic acid detection. Biosens Bioelectron 192 (2021).
39. Spiess, A. N., Mueller, N. & Ivell, R. Trehalose is a potent PCR enhancer: Lowering of DNA melting temperature and thermal stabilization of Taq polymerase by the disaccharide trehalose. Clin Chem 50, 1256-1259 (2004).
40. Jensen, M. A., Fukushima, M. & Davis, R. W. DMSO and Betaine Greatly Improve Amplification of GC-Rich Constructs in De Novo Synthesis. Plos One 5 (2010).
41. Jain, I. et al. Defining the seed sequence of the Cas12b CRISPR-Cas effector complex. Rna Biol 16, 413-422 (2019).
42. Gupta, E., Bajpai, M. & Choudhary, A. Hepatitis C virus: Screening, diagnosis, and interpretation of laboratory assays. Asian J Transfus Sci 8, 19-25 (2014).
43. Madhvi, A. et al. A screen for novel hepatitis C virus RdRp inhibitor identifies a broad-spectrum antiviral compound. Sci Rep-Uk 7 (2017).
44. Spearman, C. W., Dusheiko, G. M., Hellard, M. & Sonderup, M. Hepatitis C. Lancet 394, 1451-1466 (2019).
45. Zeuzem, S. et al. Glecaprevir-Pibrentasvir for 8 or 12 Weeks in HCV Genotype 1 or 3 Infection. N Engl J Med 378, 354-369 (2018).
46. Konerman, M. A. & Lok, A. S. Diagnostic challenges of hepatitis C. JAMA 311, 2536-2537 (2014).
47. Ishizaki, A. et al. Survey of programmatic experiences and challenges in delivery of hepatitis B and C testing in low- and middle-income countries. BMC Infect Dis 17, 696 (2017).
48. Terrault, N. A. Hepatitis C elimination: challenges with under-diagnosis and under-treatment. F1000Res 8 (2019).

We claim at least the following:

1. A genetically engineered variant BrCas12b CRISPR-associated (Cas) enzyme comprising a peptide sequence of SEQ ID NO: 1 with at least one mutation from the group consisting of: K44W, S92L, F208W, N524V, D567W, R634L, L795I, D868I, D868V, T874H, T874S, D951F, A1015E, R159E, D209R, F208W, and combinations thereof.

2. The genetically engineered variant BrCas12B enzyme of claim 1, wherein the genetically engineered variant BrCas12B enzyme has greater thermal stability, greater nuclease activity, or both, as compared to a corresponding wild type BrCas12b Cas enzyme.

3. The genetically engineered variant BrCas12B enzyme of claim 1 or 2, wherein the genetically engineered variant BrCas12B enzyme retains structural stability and has enzymatic activity at temperatures from about 55-70° C.

4. A method of detecting a target polynucleotide in a sample, the method comprising:
combining the sample in a single reaction vessel comprising:
a set of isothermal amplification components comprising isothermal amplification enzymes and primers configured to recognize and amplify the target polynucleotide;
a BrCas12b CRISPR-associated (Cas) enzyme;
an sgRNA sequence comprising a CRISPR RNA (crRNA) sequence configured to bind to the target polynucleotide and a tracrRNA sequence configured to interact with the BrCas12b Cas enzyme to form a CRISPR/Cas complex upon binding of the crRNA sequence to the target polynucleotide; and
a plurality of probes, each probe comprising an oligonucleotide element labeled with a detectable label, wherein the probe is configured to be cleaved by the BrCas12b Cas enzyme when the crRNA sequence binds the target polynucleotide to generate a CRISPR-generated detectable signal or detectable molecule;
incubating the contents of the reaction vessel at a temperature of about 60-70° C. for a period of time; and
detecting the CRISPR-generated detectable signal or detectable molecule if the target polynucleotide is present in the sample.

5. The method of claim 4, further comprising adding to the reaction vessel an isothermal amplification buffer compatible with the isothermal amplification components and the BrCas12b Cas enzyme.

6. The method of claim 4 or 5, wherein the BrCas12b Cas enzyme is Brevibacillus sp. SYP-B805 Cas12b having a peptide sequence of SEQ ID NO: 1 or a genetically engineered variant thereof.

7. The method of claim 6, wherein the BrCas12b Cas enzyme is a genetically engineered variant of Brevibacillus sp. SYP-B805 Cas12b having a higher thermostability than the wild type Brevibacillus sp. SYP-B805 Cas12b.

8. The method of claim 7, wherein the genetically engineered variant of Brevibacillus sp. SYP-B805 comprises at least one mutation from the group consisting of: K44W, S92L, F208W, N524V, D567W, R634L, L795I, D868I, D868V, T874H, T874S, D951F, A1015E, R159E, D209R, F208W, and combinations thereof.

9. The method of any of claims 4-8, wherein the set of isothermal amplification components further comprises an isothermal amplification reporter configured to produce an amplification-generated detectable signal or detectable molecule upon amplification of the target polynucleotide, wherein the amplification-generated detectable signal or detectable molecule of the isothermal amplification reporter is different and distinguishable from the CRISPR-generated detectable signal or detectable molecule produced by cleavage of the probes.

10. The method of claim 9, wherein the isothermal amplification reporter is an SYTO dye.

11. The method of any of claims 4-9, wherein the probe is selected from a FAM-polyT-Quencher (PAM-FQ) and a HEX-polyT-Quencher (HEX-FQ) reporter.

12. The method of any of claims 4-11, wherein the oligonucleotide element of the probe is a ssDNA and is about 80% of A and/or T.

13. The method of any of claims 4-12, wherein the oligonucleotide element of the probe comprises a nucleotide sequence selected from TTATT (SEQ ID NO: 3) and TTTTTTTT (SEQ ID NO: 4).

14. The method of any of claims 4-13, wherein incubating comprises incubating the contents of the reaction vessel at a temperature of about 62-68° C.

15. The method of any of claims 4-14, wherein the contents of the reaction vessel are incubated for about 8-30 minutes.

16. The method of any of claims 4-15, wherein the set of isothermal amplification components comprise loop-mediated isothermal amplification (LAMP) or reverse-transcription LAMP (RT-LAMP) enzymes and primers.

17. The method of claim 16, wherein the LAMP/RT-LAMP enzymes comprise a thermostable polymerase and wherein the LAMP/RT-LAMP primers comprise at least a forward internal primer (FIP), a backward internal primer (BIP), a forward outer primer, and a backward outer primer.

18. The method of claim 17, wherein the target polynucleotide is DNA and the set of isothermal amplification components comprise LAMP enzymes and primers and a thermostable polymerase.

19. The method of claim 17, wherein the target polynucleotide is RNA and the set of isothermal amplification components comprise RT-LAMP enzymes and primers and wherein the RT-LAMP enzymes comprise a thermostable reverse transcriptase and thermostable a polymerase.

20. The method of any of claims 4-19, wherein the target polynucleotide is a SARS-CoV-2 polynucleotide.

21. The method of claim 20, wherein the method can distinguish between variants of SARS-CoV-2.

22. The method of claim 21, wherein the variants are selected from Alpha (B.1.1.7), Beta (B.1.352), Delta (B.1.617.2), Gamma (P1).

23. The method of claim 22, wherein the target polynucleotide is a SARS-CoV-2 variant selected from Alpha (B.1.1.7), Beta (B.1.352), Delta (B.1.617.2), and Gamma (P1), the method further comprising including in the reaction vessel an isothermal amplification reporter configured to produce an amplification-generated detectable signal or molecule upon amplification of the SARS-CoV-2 polynucleotide, wherein the amplification-generated detectable signal or molecule of the isothermal amplification reporter is different and distinguishable from the CRISPR-generated detectable signal or molecule produced by cleavage of the probes, wherein detecting the amplification-generated detectable signal or molecule indicates the presence of SARS-CoV-2 in the sample, and detecting the CRISPR-generated detectable signal or molecule indicates the presence of the SARS-CoV-2 variant.

24. The method of any of claims 4-23, further comprising adding sucrose to the reaction vessel at a concentration of about 100 mM-350 mM, wherein the sucrose increases the detection capability of the BrCas12b enzyme.

25. A one-pot nucleic acid detection system for detecting a target polynucleotide in a sample, the system comprising:
a set of isothermal amplification components comprising isothermal amplification enzymes and primers configured to recognize and amplify the target polynucleotide;
a BrCas12b CRISPR-associated (Cas) enzyme;
an sgRNA sequence comprising a CRISPR RNA (crRNA) sequence configured to bind to the target polynucleotide and a tracrRNA sequence configured to interact with the BrCas12b Cas enzyme to form a CRISPR/Cas complex upon binding of the crRNA sequence to the target polynucleotide; and
a plurality of probes, each probe comprising an oligonucleotide element labeled with a detectable label, wherein the probe is configured to be cleaved by the BrCas12bCas enzyme when the guide sequence binds the target polynucleotide to generate a CRISPR-generated detectable signal or molecule.

26. The system of claim 25, further comprising a single reaction vessel configured to contain the elements of the system of claim X in a single pot and further comprising a heating element to maintain the reaction vessel at a temperature of about 60-70° C.

27. The system of claim 25 or 26, further comprising an isothermal amplification buffer compatible with the isothermal amplification components and the BrCas12b Cas enzyme.

28. The system of any of claims 25-27, wherein the BrCas12b is Brevibacillus sp. SYP-B805 Cas12b having the polypeptide sequence SEQ ID NO: 1 or a genetically engineered variant thereof.

29. The system of claim 28, wherein the BrCas12b Cas enzyme is a genetically engineered variant of Brevibacillus sp. SYP-B805 Cas12b having a higher thermostability than the wild type Brevibacillus sp. SYP-B805 Cas12b.

30. The system of claim 29, wherein the genetically engineered variant of Brevibacillus sp. SYP-B805 comprises at least one mutation from the group consisting of: K44W, S92L, F208W, N524V, D567W, R634L, L795I, D868I, D868V, T874H, T874S, D951F, A1015E, R159E, D209R, F208W, and combinations thereof.

31. The system of any of claims 25-30, wherein the set of isothermal amplification components further comprises an isothermal amplification reporter configured to produce an amplification-generated detectable signal or detectable molecule upon amplification of the target polynucleotide, wherein the amplification-generated detectable signal or detectable molecule of the isothermal amplification reporter is different and distinguishable from the CRISPR-generated detectable signal or detectable molecule produced by cleavage of the probes.

32. The system of claim 31, wherein the isothermal amplification reporter is SYTO9 dye and the probe is a HEX-FQ reporter.

33. The system of claim 32, wherein the oligonucleotide element of the probe comprises a nucleotide sequence selected from TTATT and TTTTTTTT.

34. The system of any of claims 25-33, wherein the set of isothermal amplification components comprise loop-mediated isothermal amplification (LAMP) or reverse-transcription LAMP (RT-LAMP) enzymes and primers.

35. The system of claim 34, wherein the LAMP/RT-LAMP enzymes comprise a thermostable polymerase and wherein the LAMP/RT-LAMP primers comprise at least a forward internal primer (FIP), a backward internal primer (BIP), a forward outer primer, and a backward outer primer.

36. The system of claim 35, wherein the target polynucleotide is RNA and the set of isothermal amplification components comprise RT-LAMP enzymes and primers and wherein the RT-LAMP enzymes comprise a thermostable reverse transcriptase and thermostable a polymerase.

37. The system of any of claims 25-36, wherein the target polynucleotide is a SARS-CoV-2 polynucleotide.

38. The system of claim 37, wherein the method can distinguish between variants of SARS-CoV-2.

39. The system of claim 38, wherein the target polynucleotide is a SARS-CoV-2 variants are selected from Alpha (B.1.1.7), Beta (B.1.352), Delta (B.1.617.2), and Gamma (P1), the method further comprising including in the reaction vessel an isothermal amplification reporter configured to produce an amplification-generated detectable signal or molecule upon amplification of the SARS-CoV-2 polynucleotide, wherein the amplification-generated detectable signal or molecule of the isothermal amplification reporter is different and distinguishable from the CRISPR-generated detectable signal or molecule produced by cleavage of the probes, wherein detecting the amplification-generated detectable signal or molecule indicates the presence of SARS-CoV-2 in the sample, and detecting the CRISPR-generated detectable signal or molecule indicates the presence of the SARS-CoV-2 variant.

40. A shelf-stable kit for detecting a target polynucleotide in a sample comprising the following components:
a) a set of lyophilized isothermal amplification components comprising isothermal amplification enzymes and primers configured to recognize and amplify the target polynucleotide;
b) a lyophilized BrCas12b CRISPR-associated (Cas) enzyme;
c) a lyophilized sgRNA sequence comprising a CRISPR RNA (crRNA) sequence configured to bind to the target polynucleotide and a tracrRNA sequence configured to interact with the BrCas12b Cas enzyme to form a CRISPR/Cas complex upon binding of the crRNA sequence to the target polynucleotide;
d) a plurality of lyophilized probes, each probe comprising an oligonucleotide element labeled with a detectable label, wherein the probe is configured to be cleaved by the BrCas12bCas enzyme when the crRNA sequence binds the target polynucleotide to generate a CRISPR-generated detectable signal or molecule; and
instructions for combining components a-d with a sample, incubating the sample at a temperature of about 60-70° C. for a period of time, and detecting the detectable signal or molecule.

41. The kit of claim 40, wherein the kit further comprises lyophilized isothermal amplification buffer compatible with the isothermal amplification components and the BrCas12b Cas enzyme.

42. The kit of claim 40 or 41, wherein the BrCas12b is Brevibacillus sp. SYP-B805 Cas12b having the polypeptide sequence SEQ ID NO: 1 or a genetically engineered variant thereof.
